(19) United States
(12) Patent Application Publication
(10) Pub. No.: US 2025/0101424 A1
(43) Pub. Date: Mar. 27, 2025

(54) MIRNA CIRCUITS FOR CONTROLLED GENE EXPRESSION

(71) Applicant: CALIFORNIA INSTITUTE OF TECHNOLOGY, Pasadena, CA (US)

(72) Inventors: Michael B. Elowitz, Pasadena, CA (US); Rongrong Du, Pasadena, CA (US); Michael J. Flynn, Pasadena, CA (US)

(21) Appl. No.: 18/895,366

(22) Filed: Sep. 24, 2024

Related U.S. Application Data
(60) Provisional application No. 63/540,300, filed on Sep. 25, 2023.
(60) Provisional application No. 63/563,033, filed on Mar. 8, 2024.

(51) Int. Cl. C12N 15/113; C12N 15/85

(52) U.S. Cl. CPC C12N 15/113; C12N 15/85; C12N 2310/141

(57) ABSTRACT

Disclosed herein include methods, compositions, and kits suitable for use in tuned dosage-invariant expression of a payload protein. Compositions (e.g., nucleic acid compositions, one or more cells) provided herein can comprise a first promoter sequence operably linked to a first polynucleotide comprising one or more miRNA cassettes, and a second promoter sequence operably linked to a second polynucleotide comprising a payload gene. In some embodiments, the first promoter sequence is capable of inducing transcription of the first polynucleotide to generate a first transcript, and the first transcript is capable of being processed to generate said miRNA. The payload gene can comprise a miRNA target region comprising one or more miRNA target sequences.

### RELATED APPLICATIONS

The present application claims priority to U.S. Provisional Application No. 63/540,300, filed Sep. 25, 2023; and U.S. Provisional Application No. 63/563,033, filed Mar. 8, 2024. The entire contents of these applications are hereby expressly incorporated by reference in their entireties.

### STATEMENT REGARDING FEDERALLY SPONSORED R&D

This invention was made with government support under Grant No. EB030015 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365875-US, created Sep. 24, 2024, which is 86,964 bytes in size. The information in the electronic format of the Sequence Listing is incorporated herein by reference in its entirety.

### BACKGROUND

### Field

The present disclosure relates generally to the field of polynucleotide delivery and expression.

### Description of the Related Art

The ability to express transgenes at specified levels is critical for understanding cellular behaviors, and for applications in gene and cell therapy. Transfection, viral vectors, and other gene delivery methods produce varying protein expression levels, with limited quantitative control, while targeted knock-in and stable selection are inefficient and slow. Active compensation mechanisms can improve precision, but the need for additional proteins or lack of tunability have prevented their widespread use. There is a need for compact synthetic miRNA-based regulatory circuits enabling tunable, orthogonal, and generalizable dosage-invariant gene expression control for research and biotechnology.

### SUMMARY

There are provided, in some embodiments, compositions (e.g., nucleic acid compositions, one or more cells, DIMMER circuits). Disclosed herein include nucleic acid compositions. In some embodiments, the nucleic acid composition comprises: a first promoter sequence operably linked to a first polynucleotide comprising one or more miRNA cassettes. In some embodiments, the first promoter sequence is capable of inducing transcription of the first polynucleotide to generate a first transcript. In some embodiments, the first transcript is capable of being processed to generate said miRNA. In some embodiments, the nucleic acid composition comprises: a second promoter sequence operably linked to a second polynucleotide comprising a payload gene. In some embodiments, the payload gene comprises a miRNA target region comprising one or more miRNA target sequences. In some embodiments, the second promoter sequence is capable of inducing transcription of the second polynucleotide to generate a payload transcript. In some embodiments, the payload transcript is capable of being translated to generate a payload protein. In some embodiments, the first promoter sequence and the second promoter sequence are components of a bidirectional promoter. In some embodiments, the first promoter sequence and the second promoter sequence are in reverse complementary orientation with respect to each other in the bidirectional promoter.

Disclosed herein include compositions comprising one or more cells. In some embodiments, the one or more cells comprise: a first promoter sequence operably linked to a first polynucleotide comprising one or more miRNA cassettes. In some embodiments, the first promoter sequence is capable of inducing transcription of the first polynucleotide to generate a first transcript. In some embodiments, the first transcript is capable of being processed to generate said miRNA. In some embodiments, the one or more cells comprise: a second promoter sequence operably linked to a second polynucleotide comprising a payload gene. In some embodiments, the payload gene comprises a miRNA target region comprising one or more miRNA target sequences. In some embodiments, the second promoter sequence is capable of inducing transcription of the second polynucleotide to generate a payload transcript. In some embodiments, the payload transcript is capable of being translated to generate a payload protein. In some embodiments, the first promoter sequence and the second promoter sequence are components of a bidirectional promoter. In some embodiments, the first promoter sequence and the second promoter sequence are in reverse complementary orientation with respect to each other in the bidirectional promoter.

In some embodiments, the miRNA is capable of binding the one or more miRNA target sequences, thereby reducing the stability of the payload transcript and/or reducing the translation of the payload transcript. In some embodiments, the first polynucleotide comprises a dosage gene. In some embodiments, the first transcript is capable of being translated to generate dosage indicator protein. In some embodiments, an intron is located in the dosage gene 3′UTR, dosage gene 5′UTR, or between dosage gene exons, optionally a synthetic intron. In some embodiments, the intron comprises the one or more miRNA cassettes. In some embodiments, the intron comprises one or more of: (i) an intronic insert encoding a miRNA, (ii) a donor splice site, (iii) an acceptor splice site, (iv) a branch point domain; and (v) a polypyrimidine tract. In some embodiments, the miRNA, or precursor thereof, is capable of being released from said intron by an intron excision mechanism selected from the group comprising cellular RNA splicing and/or processing machinery, nonsense-mediated decay (NMD) processing, or any combination thereof, optionally the precursor comprises a pri-miRNA. In some embodiments, the dosage indicator protein is detectable, optionally the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

In some embodiments, the one or more cells comprise two or more cells. In some embodiments, the two or more cells comprise a first cell and a second cell. In some embodiments, the first cell is a first cell type and/or the second cell is a second cell type. In some embodiments, the (i) first and second cell type and/or (ii) the one or more cells, are selected from the group comprising: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

In some embodiments, the first polynucleotide is transcribed at a rate at least 1.1-fold higher in the first cell as compared to the second cell. In some embodiments, the first polynucleotide is translated at a rate at least 1.1-fold higher in the first cell as compared to the second cell. In some embodiments, the first polynucleotide is present at a copy number at least 1.1-fold higher in the first cell as compared to the second cell. In some embodiments, the threshold dosage at which payload protein expression is saturated is at least 1.1-fold higher in the first cell as compared to the second cell. In some embodiments, the rate of transcription of the second polynucleotide and/or the rate of translation of the payload transcript varies between a first time point and a second time point in a single cell and/or varies between the first cell and the second cell at the same time point. In some embodiments, in the absence of the miRNA, the payload protein reaches untuned steady state payload protein levels in the one or more cells. In some embodiments, untuned steady state payload protein levels range between a lower untuned threshold and an upper untuned threshold of an untuned expression range. In some embodiments, steady state dosage indicator protein levels reflect untuned steady state payload protein levels. In some embodiments, in the presence the miRNA, the payload protein reaches tuned steady state payload protein levels in the one or more cells.

In some embodiments, tuned steady state payload protein levels range between a lower tuned threshold and an upper tuned threshold of a tuned expression range, optionally wherein upper tuned threshold of the tuned expression range is a saturating expression level. In some embodiments, at the first time point and the second time point in a single cell, the steady state levels of the payload protein remain within the tuned expression range. In some embodiments, in the first cell and the second cell at the same time point, the steady state levels of the payload protein remain within the tuned expression range. In some embodiments, the lower tuned threshold and/or the upper tuned threshold of a tuned expression range is capable of being configured by modulating one or more of (i) the number of miRNA cassettes within the first polynucleotide, (ii) the number of miRNA target sequences in the miRNA target region, (iii) the complementarity between the miRNA and the one or more miRNA target sequences, and (iv) strength of the first promoter sequence and/or second promoter sequence. In some embodiments, the difference between the lower untuned threshold and the upper untuned threshold of the untuned expression range is greater than about two orders of magnitude. In some embodiments, the difference between the lower tuned threshold and the upper tuned threshold of the tuned expression range is less than about one order of magnitude. In some embodiments, the payload protein is efficacious at steady state payload protein levels within the tuned expression range. In some embodiments, the payload protein is inefficacious and/or toxic at steady state payload protein levels above and/or below the tuned expression range. In some embodiments, the payload protein is capable of inducing an immunogenic response and/or a cytokine storm at steady state payload protein levels outside the tuned expression range. In some embodiments, tuned steady state payload protein levels comprise a therapeutic level of the payload protein. In some embodiments, the steady state payload protein levels remain within the tuned expression range across multiple cell types, titers of viral vector, and/or viral vector capsid types. In some embodiments, the tuned steady state payload protein levels are robust to tissue tropism and stochastic expression.

In some embodiments, the miRNA comprises a nucleotide sequence that is at least 80%, 85%, 90%, 95%, 98%, 99%, or 100% identical to SEQ ID NOs: 75-87. In some embodiments, the miRNA cassette comprises a nucleotide sequence that is at least 80%, 85%, 90%, 95%, 98%, 99%, or 100% identical to SEQ ID NOs: 1-13. In some embodiments, the miRNA is about 20, 21, 22, 23, or 24, nucleotides (nt) in length. In some embodiments, the sequence of the miRNA is orthogonal to the one or more cells, optionally a miRNA sequence targeting Renilla luciferase. In some embodiments, the miRNA target region is situated in the 3′ UTR, 5′ UTR, or coding region of the payload gene. In some embodiments, the miRNA target region comprises a nucleotide sequence that is at least 80%, 85%, 90%, 95%, 98%, 99%, or 100% identical to SEQ ID NOs: 14-74. In some embodiments, the miRNA target region comprises at least about 1, 2, 3, 4, 5, 6, 7, 8, 9, 10, 11, 12, 13, 14, 15, 16, 17, 18, 19, or 20 miRNA target sequences. In some embodiments, a payload transcript is capable of being simultaneously bound by multiple miRNA-loaded Argonaute (Ago) complexes, optionally via TNRC6. In some embodiments, 5, 6, 7, 8, 9, 10, 11, 12, 13, 14, 15, 16, 17, 18, 19, 20, 21, nucleotides (nt) of a miRNA target sequence is complementary to the miRNA, optionally 1, 2, 3, 4, 5, 6, 7, 8, 9, 10, 11, 12, 13, 14, or 15 nt of the 3′ end of the miRNA lack complementarity. In some embodiments, the complementarity between the miRNA and a miRNA target sequence is at least, or at most, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, the miRNA comprises 5-8 GC nucleotides, optionally 1-4 in the seed region and 1-2 in the extensive region. In some embodiments, the composition comprises one or more supplemental payload genes and one or more supplemental miRNA, wherein the supplemental miRNA differ in sequence with respect to each other.

In some embodiments, the composition, for each distinct supplemental miRNA, comprises: a supplemental first promoter sequence operably linked to a supplemental first polynucleotide comprising one or more supplemental miRNA cassettes, wherein the supplemental first promoter sequence is capable of inducing transcription of the supplemental first polynucleotide to generate a supplemental first transcript, wherein the supplemental first transcript is capable of being processed to generate said supplemental miRNA; and a supplemental second promoter sequence operably linked to a supplemental second polynucleotide comprising a supplemental payload gene, wherein the supplemental payload gene comprises a supplemental miRNA target region comprising one or more supplemental miRNA target sequences, wherein the supplemental second promoter sequence is capable of inducing transcription of the supplemental second polynucleotide to generate a supplemental payload transcript, wherein the supplemental payload transcript is capable of being translated to generate a supplemental payload protein.

In some embodiments, the first promoter sequence and/or second promoter sequence comprises a ubiquitous promoter, optionally the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), 0-kinesin (O-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof. In some embodiments, the first promoter sequence and/or second promoter sequence is an inducible promoter, optionally the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter. In some embodiments, the first promoter sequence and/or second promoter sequence comprises a tissue-specific promoter and/or a lineage-specific promoter. In some embodiments, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter.

In some embodiments, the tissue specific promoter is a neuron-specific promoter, optionally the neuron-specific promoter comprises a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ1.7 promoter, a Naᵥ1.8 promoter, a Naᵥ1.9 promoter, or an Advillin promoter. In some embodiments, the tissue specific promoter is a muscle-specific promoter, optionally the muscle-specific promoter comprises a creatine kinase (MCK) promoter. In some embodiments, the first promoter sequence and/or second promoter sequence is a methyl CpG binding protein 2 (MeCP2) promoter or a derivative thereof, optionally the MeCP2 promoter or a derivative thereof comprises a MeCP2 promoter truncated to about 229 bp or a MeCP2 promoter truncated to about 406 bp. In some embodiments, one or more cells comprise an endogenous version of the payload gene, and wherein the promoter comprises or is derived from the promoter of the endogenous version.

In some embodiments, a payload protein comprises a disease-associated protein, wherein aberrant expression of the disease-associated protein correlates with the occurrence and/or progression of the disease. In some embodiments, a payload protein comprises a protein associated with an expression-sensitive disease or disorder. In some embodiments, a payload protein comprises methyl CpG binding protein 2 (MeCP2), SMN, DRK1A, KAT6A, NIPBL, HDAC4, UBE3A, EHMT1, one or more genes encoded on chromosome 9q34.3, NPHP1, LIMK1 one or more genes encoded on chromosome 711.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof. In some embodiments, a payload protein comprises an imaging agent, optionally wherein the imaging agent comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof, optionally the imaging agent is associated with an endogenous protein or exogenous protein. In some embodiments, a payload protein comprises an endogenous protein or exogenous protein associated with a binding domain, optionally a binding domain configured for isolation or an imaging. In some embodiments, a payload protein comprises a programmable nuclease, optionally the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, CasT, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, prime editing versions thereof, or any combination thereof. The composition can comprise: a polynucleotide encoding (i) a targeting molecule and/or (ii) a donor nucleic acid. In some embodiments, the targeting molecule is capable of associating with the programmable nuclease, optionally wherein the targeting molecule comprises single strand DNA or single strand RNA, further optionally wherein the targeting molecule comprises a single guide RNA (sgRNA). In some embodiments, a payload protein is capable of modulating the expression, concentration, localization, stability, and/or activity of the one or more endogenous proteins of a cell. In some embodiments, a payload protein is a therapeutic protein or a variant thereof, optionally a therapeutic protein configured to prevent or treat a disease or disorder of a subject, further optionally the subject suffers from a deficiency of said therapeutic protein.

In some embodiments, a payload protein comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, a payload protein comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. In some embodiments, a payload protein comprises a CRE recombinase, GCaMP, a cell therapy component, a knock-down gene therapy component, a cell-surface exposed epitope, or any combination thereof. In some embodiments, a payload protein comprises a bispecific T cell engager (BiTE). In some embodiments, a payload protein comprises a cytokine, optionally the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof. In some embodiments, a payload protein comprises a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. In some embodiments, a payload protein comprises a member of the TNF family of cytokines selected from the group consisting of TNF-alpha, TNF-beta, LT-beta, CD40 ligand, Fas ligand, CD 27 ligand, CD 30 ligand, and 4-1 BBL. In some embodiments, a payload protein comprises a member of the immunoglobulin superfamily of cytokines selected from the group consisting of B7.1 (CD80) and B7.2 (B70). In some embodiments, a payload protein comprises an interferon, optionally the interferon is selected from interferon alpha, interferon beta, or interferon gamma. In some embodiments, a payload protein comprises a chemokine, optionally the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. In some embodiments, a payload protein comprises an interleukin, optionally the interleukin is selected from IL-10 IL-12, IL-1, IL-6, IL-7, IL-15, IL-2, IL-18 or IL-21. In some embodiments, a payload protein comprises a tumor necrosis factor (TNF), optionally the TNF is selected from TNF-alpha, TNF-beta, TNF-gamma, CD252, CD154, CD178, CD70, CD153, or 4-1BBL. In some embodiments, a payload protein comprises a factor locally down-regulating the activity of endogenous immune cells. In some embodiments, a payload protein is capable of remodeling a tumor microenvironment and/or reducing immunosuppression at a target site of a subject.

In some embodiments, a payload protein comprises a chimeric antigen receptor (CAR) or T-cell receptor (TCR). In some embodiments, the CAR and/or TCR comprises one or more of an antigen binding domain, a transmembrane domain, and an intracellular signaling domain. In some embodiments, the intracellular signaling domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12, or a functional variant thereof. In some embodiments, the costimulatory domain comprises a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof. In some embodiments, the antigen binding domain binds a tumor antigen, optionally the tumor antigen is a solid tumor antigen. In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HMI.24, HMW-MAA, Her2, Her2/neu, IGF-IR, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (DI), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens. In some embodiments, the antigen binding domain comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the antigen binding domain is connected to the transmembrane domain by a hinge region. In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In some embodiments, the CAR or TCR further comprises a leader peptide. In some embodiments, the TCR further comprises a constant region and/or CDR4.

In some embodiments, a payload protein is an activity regulator, optionally the activity regulator is capable of reducing T cell activity. In some embodiments, the activity regulator comprises a ubiquitin ligase involved in TCR/CAR signal transduction selected from the group comprising c-CBL, CBL-B, ITCH, R F125, R F128, WWP2, or any combination thereof. In some embodiments, the activity regulator comprises a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. In some embodiments, the activity regulator is a negative regulatory scaffold/adapter protein selected from the group comprising PAG, LIME, NTAL, LAX31, SIT, GAB2, GRAP, ALX, SLAP, SLAP2, DOK1, DOK2, or any combination thereof. In some embodiments, the activity regulator is a dominant negative version of an activating TCR signaling component selected from the group comprising ZAP70, LCK, FYN, NCK, VAV1, SLP76, ITK, ADAP, GADS, PLCgammal, LAT, p85, SOS, GRB2, NFAT, p50, p65, API, RAPI, CRKII, C3G, WAVE2, ARP2/3, ABL, ADAP, RIAM, SKAP55, or any combination thereof. In some embodiments, the activity regulator comprises the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof. In some embodiments, the activity regulator is targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

In some embodiments, a payload protein is a cellular reprogramming factor capable of converting an at least partially differentiated cell to a less differentiated cell, optionally Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof. In some embodiments, a payload protein is a cellular reprogramming factor capable of differentiating a given cell into a desired differentiated state, optionally nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof. In some embodiments, a payload protein comprises an agonistic or antagonistic antibody or antigen-binding fragment thereof specific to a checkpoint inhibitor or checkpoint stimulator molecule, optionally PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3. In some embodiments, a payload protein comprises a constitutive signal peptide for protein degradation, optionally PEST. In some embodiments, a payload protein comprises a nuclear localization signal (NLS) or a nuclear export signal (NES). In some embodiments, a payload protein comprises a synthetic protein circuit component, optionally a synthetic protein circuit component comprises a transcription factor, protease, kinase, phosphatase, a Synthetic Notch (SynNotch) receptor, a Modular Extracellular Sensor Architecture (MESA) receptor, Tango, or dCas9-synR. In some embodiments, a payload protein comprises a degron and a cut site a protease is capable of cutting to expose the degron, and wherein the degron of the payload protein being exposed changes the payload protein to a payload protein destabilized state, optionally the degron comprises an N-degron, a dihydrofolate reductase (DHFR) degron, a FKB protein (FKBP) degron, derivatives thereof, or any combination thereof. In some embodiments, a payload protein comprises a protease or a split protease, optionally the activation level of the protease is related to one or more input signals, further optionally the protease comprises tobacco etch virus (TEV) protease, tobacco vein mottling virus (TVMV) protease, hepatitis C virus protease (HCVP), derivatives thereof, or any combination thereof.

In some embodiments, a payload protein is associated with an agricultural trait of interest selected from the group consisting of increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof. In some embodiments, a payload protein is associated with a biological manufacturing process selected from the group comprising fermentation, distillation, biofuel production, production of a compound, production of a polypeptide, or any combination thereof.

In some embodiments, a payload protein comprises a synthetic receptor, optionally a Synthetic Notch (SynNotch) receptor, a Modular Extracellular Sensor Architecture (MESA) receptor, Tango, dCas9-synR, or any combination thereof. In some embodiments, a payload protein comprises a component of a RNA export system, a lipid-enveloped nanoparticle (LN) production system, or a virus-like particle (VLP) production system, optionally an RNA exporter protein or a fusogen. In some embodiments, the nucleic acid composition comprises less than about 1.0 kb, 1.5 kb, 2.0 kb, 2.5 kb, 3.0 kb, 3.5 kb, 4.0 kb, 4.5 kb, 5.0 kb, 5.5 kb, 6.0 kb, 6.5 kb, 7.0 kb, 7.5 kb, 8.0 kb, 8.5 kb, 9.0 kb, 9.5 kb, 10 kb, 12 kb, 15 kb, 20 kb, 25 kb, 30 kb, 40 kb, 50 kb, 60 kb, 70 kb, 80 kb, 90 kb, or 100 kb. In some embodiments, the expression of the miRNA perturbs endogenous gene expression of the one or more cells less that about 10%. The method can comprise: one or more nucleic acids encoding TNRC6, GW182, one or more miRNA biogenesis components, Exportin-5, Dicer, and/or one or more Argonaute proteins. In some embodiments, the in the absence of a recombination event, the first promoter sequence and the first polynucleotide are not operably linked, and wherein the first promoter sequence and the first polynucleotide are operably linked after the recombination event such that the first promoter sequence is capable of inducing transcription of the first polynucleotide to generate a first transcript. In some embodiments, the first polynucleotide and the second polynucleotide are integrated in the genome of the one or more cells.

In some embodiments, the composition is a vector, a ribonucleoprotein (RNP) complex, a liposome, a nanoparticle, an exosome, a microvesicle, or any combination thereof. In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and In some embodiments, the transposable element is piggybac transposon or sleeping beauty transposon. In some embodiments, the first polynucleotide and the second polynucleotide are comprised in the one or more vectors. In some embodiments, the first polynucleotide and the second polynucleotide are comprised in the same vector and/or different vectors. In some embodiments, the first polynucleotide and the second polynucleotide are situated on the same nucleic acid and/or different nucleic acids. In some embodiments, the viral vector is an AAV vector, a lentivirus vector, a retrovirus vector, or an integration-deficient lentivirus (IDLV) vector. In some embodiments, the AAV vector comprises single-stranded AAV (ssAAV) vector or a self-complementary AAV (scAAV) vector. In some embodiments, the AAV vector comprises AAV1, AAV2, AAV3, AAV4, AAV5, AAV6, AAV7, AAV8, AAV9, derivatives thereof, or any combination thereof. In some embodiments, the AAV vector comprises an AAV9 variant engineered for systemic delivery, optionally AAV-PHP.B, AAV-PHP.eB, or AAV-PHP.S. In some embodiments, the vector is a neurotropic viral vector, optionally wherein the neurotropic viral vector comprises or is derived from Herpesviridae, varicella zoster virus, pseudorabies virus, cyromegalovirus, Epstein-barr virus, encephalitis virus, polio virus, coxsackie virus, echo virus, mumps virus, measles virus, rabies virus, or any combination thereof.

Disclosed herein include methods of treating a disease or disorder in a subject. In some embodiments, the method comprises: administering to the subject an effective amount of one or more cells disclosed herein, thereby treating or preventing the disease or disorder in the subject.

Disclosed herein include methods of treating a disease or disorder in a subject. In some embodiments, the method comprises: introducing into one or more cells a nucleic acid composition disclosed herein; and administering to the subject an effective amount of the resulting cells, thereby treating or preventing the disease or disorder in the subject. The method can comprise: isolating the one or more cells from the subject prior to the introducing step.

Disclosed herein include methods of treating a disease or disorder in a subject. In some embodiments, the method comprises: administering to the subject an effective amount of a nucleic acid composition disclosed herein, thereby treating or preventing the disease or disorder in the subject.

Disclosed herein include methods for tuned dosage-invariant expression of a payload protein in one or more cells. In some embodiments, the method comprises: introducing into one or more cells a nucleic acid composition disclosed herein.

In some embodiments, the introducing step is performed in vivo, in vitro, and/or ex vivo. In some embodiments, the introducing step comprises calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the one or more cells comprise one or more cells of a subject, optionally the subject is suffering from a disease or disorder. In some embodiments, the one or more cells comprise a neuron, optionally the neuron is associated with a neurological disease or disorder. In some embodiments, the administering comprises systemic administration, optionally the systemic administration is intravenous, intramuscular, intraperitoneal, or intraarticular. In some embodiments, administering comprises intrathecal administration, intracranial injection, aerosol delivery, nasal delivery, vaginal delivery, rectal delivery, buccal delivery, ocular delivery, local delivery, topical delivery, intracistemal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, administering comprises an injection into a brain region, further optionally administering comprises direct administration to the brain parenchyma.

The method can comprise: introducing an inducer of the inducible promoter to the one or more cells, optionally the inducer comprises doxycycline, further optionally the introducing step comprises administering an initial dose of the inducer followed one or more lower maintenance doses of the inducer. In some embodiments, the disease or disorder comprises a MECP2-related disorder selected from the group comprising Classic Rett Syndrome, MECP2-related Severe Neonatal Encephalopathy, PPM-X Syndrome, or any combination thereof. In some embodiments, the disease or disorder is a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof.

In some embodiments, the disease or disorder comprises a neurological disease or disorder, optionally the neurological disease or disorder comprises Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

In some embodiments, the disease or disorder is an expression-sensitive disease or disorder, optionally the expression-sensitive disease or disorder is selected from the group comprising Rett Syndrome; Angelman's syndrome; spinal muscular atrophy; Smith-Magenis Syndrome; Phelan-McDermid Syndrome; Cornelia de Lange Syndrome and other NIPBL related disorders; DRK1A, KAT6A and related disorders of severe intellectual disability; Chromosome 2Q37 Deletion Syndrome and other HDAC4 Related Disorders; Angelman Syndrome; Kleefstra Syndrome; Joubert Syndrome and other NPHP1 Related Disorders; Williams Syndrome; Neurofibromatosis Type 1; Li-Fraumeni syndrome and similar p53-related cancer syndromes; Phosphofructokinase Deficiency; X-linked Hyper IgM Syndrome and similar primary immunodeficiency disorders; Triosephosphate isomerase deficiency; Kallman Syndrome; Aromatase Deficiency; Batten Disease, Frontotemporal Dementia and other neurodegenerative disorders related to loss of progranulin; Cholinergic Receptor Nicotinic Alpha 7 Subunit Related Disorders; and Hereditary Neuropathy with liability to Pressure Palsies.

In some embodiments, an expression-sensitive disease or disorder is characterized by decreased expression of one or more proteins, wherein ectopic overexpression of said one or more proteins at a steady state level beyond the upper tuned threshold causes cellular toxicity and/or disease. In some embodiments, said expression-sensitive disorder is a neurodevelopmental syndromic disorder, optionally the neurodevelopmental syndromic disorder is selected from the group comprising Rett Syndrome, Smith-Magenis Syndrome (RA1), Phelan-McDermid Syndrome (SHANK3), Comelia de Lange Syndrome (NIPBL) and other NIPBL related disorders, DRK1A, KAT6A and related disorders of severe intellectual disability, Chromosome 2Q37 Deletion Syndrome and other HDAC4 Related Disorders, Angelman Syndrome, Kleefstra Syndrome, Joubert Syndrome and other NPHP1 Related Disorders, and Williams Syndrome. In some embodiments, said expression-sensitive disorder is a proliferative disorder and/or cancer, optionally the proliferative disorder and/or cancer is selected from the group comprising Neurofibromatosis Type 1 and Li-Fraumeni syndrome and similar p53-related cancer syndromes. In some embodiments, said expression-sensitive disorder is a glycogen storage disorder, optionally the glycogen storage disorder is phosphofructokinase deficiency. In some embodiments, said expression-sensitive disorder is a hematologic disorder and/or immune disorder, optionally the hematologic disorder and/or immune disorder is selected from the group comprising X-linked Hyper IgM Syndrome and related primary immunodeficiency disorders, and triosephosphate isomerase deficiency. In some embodiments, said expression-sensitive disorder is an endocrine disorder, optionally the endocrine disorder is selected from the group comprising Kallman Syndrome and Aromatase Deficiency. In some embodiments, said expression-sensitive disorder is a neuropsychiatric disorder, optionally the neuropsychiatric disorder is selected from the group comprising Batten Disease, Frontotemporal Dementia and other neurodegenerative disorders related to loss of progranulin, Cholinergic Receptor Nicotinic Alpha 7 Subunit Related Disorders, and Hereditary Neuropathy with liability to Pressure Palsies.

In some embodiments, the payload protein comprises RA1 and the disease or disorder comprises Smith-Magenis Syndrome. In some embodiments, the payload protein comprises SHANK3 and the disease or disorder comprises Phelan-McDermid Syndrome. In some embodiments, the payload protein comprises CLN3 and the disease or disorder comprises Batten Disease. In some embodiments, the payload protein comprises NF-1 and the disease or disorder comprises Neurofibromitosis Type I. In some embodiments, the payload protein comprises TP53 and the disease or disorder comprises Li-Fraumeni Syndrome. In some embodiments, the payload protein comprises PFK and the disease or disorder comprises phosphofructokinase deficiency. In some embodiments, the payload protein comprises CD40LG and the disease or disorder comprises X-linked Hyper IGM disorders.

Disclosed herein include research methods. In some embodiments, the research method comprises: introducing into one or more cells a nucleic acid composition disclosed herein; and obtaining biological information of the one or more cells, optionally via sequencing or imaging. In some embodiments, the imaging comprises CRISPR imaging and/or super-resolution imaging, optionally DNA-PAINT (Point Accumulation for Imaging in Nanoscale Topography).

Disclosed herein include production methods. In some embodiments, the production method comprises: introducing into one or more cells a nucleic acid composition disclosed herein; and isolating the payload protein or a payload associated therewith.

### BRIEF DESCRIPTION OF THE DRAWINGS

FIGS. 1A-1C depict non-limiting exemplary schematics related to miRNA incoherent feedforward circuits enabling dosage-invariant gene expression. (FIG. 1A) An ideal gene expression system generates uniform protein expression levels despite variable gene dosage delivered. The blue gradient shown in the nucleus indicates the copy numbers of gene delivered, in which darker blue represents higher dosage, and lighter blue represents lower dosage. Green indicates the desired uniform output protein expression levels. (FIG. 1B) The architecture of the incoherent feedforward loop (IFFL). The input is gene dosage in arbitrary units, which activates the expression of both the mRNA and the microRNA. microRNA inhibits mRNA translation. Output is the resulting protein expression. (FIG. 1C) IFFLs enable tunable (Circled 1), orthogonal (Circled 2) control of the target and also operate in multiple cell contexts (Circled 3). Circled 1 shows the modeling of the dosage compensation system which permits tuning of the setpoint levels. Circled 2 depicts schematic flow cytometry data of two genes of interest regulated by orthogonal, tunable, miRNA-controlled dosage compensation circuits. Cells are poly-transfected with two independently-regulated constructs. Each color represents one set of the designs used in the poly-transfection. Each ellipse indicates where most cells are located. Dashed lines indicate the centroids of expression. Circled 3 schematically depicts relatively similar expression behavior across diverse cell types. The central diagram introduces the circuit architecture of the microRNA (miRNA)-based IFFL, in which the two arrows indicate the divergent promoter, the small rectangle on the left indicates the miRNA, and the long rectangle on the right indicates the regulated gene, the short rectangle on the right indicates the miRNA binding sites.

FIGS. 2A-2I depict non-limiting exemplary schematics and data related to miRNA-based IFFLs achieving linear regulation and dosage independence through TNRC6-mediated repression. (FIG. 2A) To identify the parameter regimes allowing dosage compensation, a simplified model of miRNA inhibition was built. Upper panel, gray dashed arrows in the diagram represent the natural dilution and degradation of the mRNA or miRNA. Black, double-directed arrows represent the association and dissociation of the mRNA-miRNA complex. Gray arrows represent the decay of the mRNA. The rates of all the reactions are labeled on the side of the arrows. Lower panel, modeling results of the miRNA-mRNA interaction. The unregulated curve suggests the expression when there's no miRNA. (FIG. 2B) To implement the miRNA-based dosage compensation system, the divergent promoter circuit was designed. The target-miRNA complementarity and target site numbers were varied to explore the parameter space that gives rise to the dosage independence. (FIG. 2C) Flow cytometry was performed to verify the behavior of the circuit shown in FIG. 2A with a single, fully complementary miR-L target site versus an unregulated control, which has neither the miR-L nor the miR-L target site. mRuby3 fluorescent protein was used as the dosage indicator, and EGFP fluorescent protein as the target. Cells are gated and binned by mRuby3 intensities. Each dot corresponds to the geometric mean fluorescence intensity of the mRuby3 bin breaks and median fluorescence intensity of EGFP in the bin. Shaded regions denote the range from ((EGFG median)/(geometric standard deviation)) to (EGFG median)×(geometric standard deviation). The gray curve shows an mRuby3-only transfection control, which suggests the bleedthrough from the mRuby3 channel to the EGFP channel. (FIG. 2D) Flow cytometry was performed to understand how the complementarity of a single target site affects the regulation. Single miR-L target sites with the complementarity ranging from 17 bp to 21 bp, starting from the seed region, were used in this experiment. mRuby3 serves as the dosage indicator, and EGFP is the target. Cells are gated and binned by mRuby3 intensities. Dots and shaded regions were calculated as described in FIG. 2C. The gray curve shows an mRuby3-only transfection control. (FIG. 2E) Flow cytometry was performed to dissect the effect of increasing target repeats while keeping the complementarity constant. A miR-L 17 bp complementary site starting from the seed region was repeated from 1 to 4 times. Cells are gated and binned by mRuby3 intensities, a constitutively expressed iRFP plasmid was co-transfected as a cotransfection marker. Dots and shaded regions were calculated as described in FIG. 2C. (FIG. 2F) A zoom-in of the 4×17 bp construct used in FIG. 2E. p.d.f, probability distribution function, which measured the distribution of mRuby3 fluorescence intensity. Cells are gated and binned by mRuby3 intensities. Dots and shaded regions were calculated as described in FIG. 2C. The gray rectangle in the scope from 10⁵ to 2×10⁶ (shown by the vertical dashed lines) on the mRuby3 axis indicates the range where the fluorescence intensity of EGFP does not change. The light gray rectangle in the scope from 5×10⁴ to 10⁷ (shown by the vertical dashed lines) on the mRuby3 axis indicates the range where the fluorescence intensity of EGFP has minor change (i.e., less than 5 fold). The dashed lines illustrate the curves with a slope of either 1 or 0, revealing either the linear dependence of the dosage or independence of the dosage. (FIG. 2G) A diagram of the T6B peptide inhibition mechanism. T6B is a short, dominant-negative version of the TNRC6B. T6B competes with TNRC6 by binding to the Ago2-miRNA complex. (FIG. 2H) To unravel the mechanism of the inhibition that gives rise to the dosage compensation behavior, cells were co-transfected with or without T6B peptide and the 4×17 bp construct described in FIG. 2F and performed flow cytometry. The T6B peptide is fused to the iRFP fluorescence protein, and the control group was co-transfected with the constitutively expressed iRFP. Cells are gated and binned by mRuby3 intensities. Dots and shaded regions were calculated as described in FIG. 2C. T6B transfected cells lost the inhibition, as illustrated in the small graph located in the plot. (FIG. 2I) To compare the mechanism of the strong inhibition with the TNRC6-dependent inhibition, cells were co-transfected with or without T6B peptide and the single site, fully complementary construct described in FIG. 2C and performed flow cytometry. The experimental setting is the same as described in FIG. 2H. Cells are gated and binned by mRuby3 intensities. Dots and shaded regions were calculated as described in FIG. 2C. T6B brought minor effects to the inhibition, and therefore suggested that the inhibition is TNRC6-independent, as illustrated in the small graph located in the plot.

FIGS. 3A-3E depict non-limiting exemplary schematics and data related to sequence complementarity, miRNA cassette copy number, and promoter strength tuning expression level. (FIG. 3A) Circuits shown in FIG. 2B were designed and constructed harboring miR-L targeting sites with the complementarity ranging from 8 bp to 21 bp starting from the seed region, with each targeting site repeated four times. In the left panel, flow cytometry was performed on the cells and each dot represents one well. Cells were gated based on the mRuby3 fluorescence intensity. Median fluorescence intensities of the EGFP in the mRuby3 positive cells were plotted. In the right panel, the dosage response curves of the five selected constructs (dashed boxes) from the left panel are displayed. Gray rectangle indicates the gated region of the left panel. Cells were binned by mRuby3 intensities. Dots were calculated as described in FIG. 2C. (FIG. 3B) Flow cytometry was performed on the cells transfected with the circuits shown in FIG. 2B harboring miR-L targeting sites with the complementarity ranging from 17 bp to 19 bp starting from the seed region, with each targeting site repeated four times. Cells were gated and binned by mRuby3 intensities. Dots and shaded regions were calculated as described in FIG. 2C. Black solid horizontal lines denote the setpoint levels of the constructs. (FIG. 3C) Flow cytometry was performed on the cells transfected with the circuits shown in FIG. 2B harboring miR-L cassettes with 1-3 repeats incorporated in the same intron, the target sites of which were miR-L targeting sites with 17 bp complementarity starting from the seed region, repeated four times. Cells were gated and binned by mRuby3 intensities. Dots and shaded regions were calculated as described in FIG. 2C. Black solid horizontal lines denote the setpoint levels of the constructs. (FIG. 3D) Flow cytometry was performed on the cells transfected with the circuits shown in FIG. 2B harboring miR-L cassettes expressed from the EF1α or PGK promoter, the target sites of which were miR-L targeting sites with 17 bp complementarity starting from the seed region, repeated four times. Cells were gated and binned by mRuby3 intensities. Dots and shaded regions were calculated as described in FIG. 2C. Black solid horizontal lines denote the setpoint levels of the constructs. The barplot at the bottom right corner shows the mRuby3 median fluorescence intensities of the transfected cells. (FIG. 3E) Upper panel, design of the 4-epi-Tetracycline inducible DIMMER. The promoter that drives the EGFP is a CMV promoter harboring two TetO sites. When expressing the constructs in the TRex cell line, the TetR transcriptional repressor binds to TetO and shuts off the transcription. 4-epi-tetracycline removes the TetR from TetO in a gradient-dependent manner, and therefore restores the transcription. Lower panel, Flow cytometry was performed on the TRex cell line which was transfected with the 4×19 bp DIMMER construct, and the EGFP was driven by the CMV-2×TetO promoter. The concentrations of the 4-epi-Tetracycline, from purple to yellow, were 0, 10, 33.3, 100, 333.3 ng/mL. The gray curve denotes the mRuby-only transfection control, and the 4-epi-Tetracycline concentration is 333.3 ng/mL.

FIGS. 4A-4H depict non-limiting exemplary schematics and data related to Algorithm-based miRNA designs being orthogonal to each other, and enabled independent regulation of multiple genes. (FIG. 4A) Diagram of the open loop system. miRNA is expressed from the intron of the 3′UTR of the mTagBFP2. mRuby3 serves as the dosage indicator, while EGFP is the target. (FIG. 4B) Flow cytometry was performed on the cells co-transfected with the circuit shown in (A) with or without the miR-L (the BFP only control does not have the 3′UTR miRNA), the target site of which is a single, fully complementary miR-L site. Cells were gated by mRuby3 and mTagBFP2 intensities and binned by mTagBFP2 intensities. The relative expression was defined as the median of the ratio of EGFP fluorescence intensity to mRuby3 fluorescence intensity in the mTagBFP2 bin. Dots and shaded regions were calculated as described in FIG. 2C. (FIG. 4C) Diagram of a mature synthetic miRNA and the sequence design constraints used to design them. (FIG. 4D) Flow cytometry was performed on the cells co-transfected with the circuit shown in FIG. 4A with the synthetic miRNAs, and the corresponding target site of which is a single, fully complementary synthetic miRNA site. The BFP only control does not have the 3′UTR miRNA and uses a target without any target sites. Cells were gated by mRuby3 and mTagBFP2 intensities and binned by mTagBFP2 intensities. Relative expression levels were quantified as described in FIG. 4B. Dots were calculated as described in FIG. 2C. (FIG. 4E) To test if the synthetic miRNAs and their targets are orthogonal to each other, the cells were poly-transfected with all the combinations of the 10 miRNAs and the 10 single, fully complementary sites (10×10 combinations) and performed flow cytometry. Each grid represents one well. Cells were gated by mRuby3 and mTagBFP2 intensities. Relative expression levels were first quantified in the transfected cells as described in FIG. 4B, and then normalized by dividing the minimal values that were larger than 1. (FIG. 4F) A gallery of the circuits with the architecture shown in FIG. 2B established based on different miRNA and targets. The repeat number and the complementarity between the target and the miRNA were denoted by m×n bp, where m represents the repeat number and n represents the complementarity. Cells were measured by flow cytometry, gated and binned by mRuby3 intensities. Dots were calculated as described in FIG. 2C. (FIG. 4G) Diagram of a double IFFL reporter system. In addition to the construct illustrated in FIG. 2B, which uses mRuby3 as the dosage indicator and EGFP as the target, a distinct construct was introduced which uses mTagBFP2 as the dosage indicator and emiRFP670 as the target. These two reporters were designed to be established on orthogonal miRNA and targets to implement independent regulations. (FIG. 4H) Flow cytometry was performed on cells that were poly-transfected with the double reporter system shown in FIG. 4G. Cells were gated by mRuby3 and mTagBFP2 intensities. The miRNA and target designs of the double reporter system are listed on the top right corner of the plot, the nomenclature of which is described in FIG. 4F, where the former part denotes the cassette regulating EGFP, and the latter part denotes the cassette regulating iRFP. The solid contour lines denote where 80% of the cell masses were located centering the expression centroids.

FIGS. 5A-5F depict non-limiting exemplary schematics and data related to DIMMER circuits working across different cell lines and not perturbing the transcriptome. (FIG. 5A) The circuit described in FIG. 2B regulated by the miR-L with the 4×17 bp target was transfected into different cell lines and performed flow cytometry. Cells were gated and binned by mRuby intensities. Dots and shaded regions were calculated as described in FIG. 2C. Gray vertical lines intersecting the x axis mark the mRuby3 fluorescence intensities where the circuit started to show dosage independence behavior. Gray horizontal lines intersecting the y axis suggest the EGFP fluorescence intensities of the setpoints. (FIG. 5B) The values of the intersection points of the gray lines with the axis in FIG. 5A were plotted as scatters. The black solid line indicates a linearity between the threshold level and the saturating level, which was described by y=k x, where k was fitted by applying a linear regression model. (FIG. 5C) Workflow of the DIMMER mono-clone sorting and measurement. (FIG. 5D) Flow cytometry was performed on the DIMMER mono-clones that showed varied mRuby distributions. The histograms of the mRuby3 and the EGFP intensities of each mono-clone are displayed on the top and the right of the scatter plot, respectively. (FIG. 5E) Bulk RNAseq was performed on the cells that were transfected with the BFP-miRNA construct. The orange and red dots depict the transcripts of the synmiR-3 expressing cells, while the light blue and dark blue dots show the transcripts of the synmiR-L expressing cells. The space out of the dashed black lines indicate where the absolute value of log 2 fold change was larger than 2. The space above the red dashed line indicates where the adjusted p-value is less than 0.05. The Venn diagram above denotes the shared and nonshared significantly differentially expressed genes between synmiR-3 and synmiR-L. (FIG. 5F) The normalized transcripts per million (TPM) of the miR-L expressing cells were plotted against those of the synmiR-3 expressing cells. Solid line indicates where the TPM of the two samples were equal to each other. Dashed lines indicate where the TPM of the synmiR-3 expressing cells is 10 fold of synmiR-L expressing cells, or where the TPM of the latter is 10 fold of the former.

FIGS. 6A-6H depict non-limiting exemplary schematics and data related to DIMMER circuits improving fluorescence imaging. (FIG. 6A) Diagram of the EGFR membrane receptor imaging experiment. The regulated EGFR-mEGFP is expected to show a more homogenous distribution of fluorescence signals. (FIG. 6B) Representative fluorescence images of the EGFR-mEGFP with (right) or without (left) the DIMMER regulation module. miR-L cassette was chosen to build the circuit for cloning convenience. Target site nomenclature was described in FIG. 4F. Numbers on the colorbars indicate the fluorescence intensities measured by imageJ. (FIG. 6C) Illustration of EGFR in the cell membrane (adapted from PDB 7SYE). EGFR is linked to an mEGFP which is labeled with a DNA-conjugated anti-GFP nanobody (PDB 6LR7). (FIG. 6D) Diffraction-limited and DNA-PAINT image of CHO-K1 cells expressing EGFR-mEGFP. The mEGFP is labeled with DNA-conjugated anti-mEGFP nanobodies. The right panel shows the zoom-in of the boxed area. (FIG. 6E) Representative single-protein-resolved DNA-PAINT images of cells transfected with EGFR-mEGFP with or without the DIMMER module. miR-L cassette was used, and the target site nomenclature was described in FIG. 4F. (FIG. 6F) Quantitation of the receptor density for the cells transfected with EGFR-mEGFP with or without the DIMMER module. Insert shows zoom-in of the cells transfected with the DIMMER circuit-regulated EGFR-mEGFP. (FIG. 6G) Diagram of the dCas9 telomere imaging experiment. The protein controlled by the circuit was switched to dCas9-EGFP. The gRNA is targeted to the telomeres and expressed from the same vector as dCas9-EGFP, but is driven by the U6 promoter. (FIG. 6H) Upper panel, Representative fluorescence images of the dCas9-EGFP with or without the DIMMER module. miR-L cassette was chosen to build the circuit for cloning convenience. Target site nomenclature was described in FIG. 4F. Numbers on the color bars indicate the fluorescence intensities measured by imageJ. Lower panel, quantitation of the relative signal intensity of the dots in the cells transfected with the dCas9-EGFP with or without the DIMMER module. Each line represents one dot inside a cell. It should be noted that it's extremely hard to find dots inside the cells transfected with the unregulated dCas9-EGFP, therefore, only three dots in one cell were found and analyzed among all the cells in the scope.

FIGS. 7A-7C depict non-limiting exemplary data related to modeling of the IFFL. (FIG. 7A) Modeling the steady-state, non-dimensionalized mRNA concentration under different k_{off} while maintaining kₒₙ=2×10⁵, k_{c}=160. (FIG. 7B) Modeling the steady-state, non-dimensionalized mRNA concentration under different k_{c} while maintaining kₒₙ=2×10⁵, k_{off}=4×10³. (FIG. 7C) Modeling the steady-state, non-dimensionalized mRNA concentration under different Hill coefficient n while maintaining K=1.

FIGS. 8A-8H depict non-limiting exemplary schematics and data related to comparisons between circuit configurations in the single transcript and the double transcript design. (FIG. 8A) The diagram of the initial design of the miR-L 4×17 bp circuit (upper) and the miRNA detector circuit (lower). The miRNA detector has the corresponding miRNA target site on the 3′UTR of the iRFP. (FIG. 8B) Flow cytometry was performed on the cells transfected with the initial design of the miR-L 4×17 bp circuit displayed in the top panel of FIG. 8A. Cells were gated and binned by mRuby3 intensities. Dots and shaded regions were calculated as described in FIG. 2C. The initial design shows a very low EGFP expression level. (FIG. 8C) Flow cytometry was performed on the cells transfected with the 4×17 bp miRNA detector alone (FIG. 8A, bottom) or with the initial miR-L 4×17 bp circuit (FIG. 8A, top). Cells were gated and binned by mTagBFP2 intensities. Dots and shaded regions were calculated as described in FIG. 2C. The detector of the initial circuit just shows very weak inhibition at very high dosage, and this inconsistency in inhibition suggests that the low expression EGFP level of the initial circuit doesn't come from miRNA inhibition. (FIG. 8D) Flow cytometry was performed on the cells transfected with the initial miR-L 4×17 bp circuit, or the initial miR-L 4×17 bp circuit along with the 4×17 bp miRNA detector. Cells were gated and binned by mRuby3 intensities. Dots and shaded regions were calculated as described in FIG. 2C. The addition of the detector doesn't affect the response of the initial miR-L 4×17 bp circuit. (FIG. 8E) Flow cytometry was performed on the cells transfected with either the initial miR-L 4×17 bp circuit or the double transcript design illustrated in FIG. 2A. Cells were gated and binned by mRuby3 intensities. Dots and shaded regions were calculated as described in FIG. 2C. (FIG. 8F) The diagram of the new single transcript design of the miR-L 4×17 bp circuit. The intronic miR-L cassette was shifted to the 5′ to the target site. (FIGS. 8G-H) Flow cytometry was performed on the cells co-transfected with the double transcript miR-L 4×17 bp circuit described in FIG. 2A and the 10×21 bp miRNA detector, versus the new single transcript design of the miR-L 4×17 bp shown in (FIG. 8F) and the 10×21 bp miRNA detector. Compared to the 4×17 bp miRNA detector, the 10×21 bp miRNA detector is able to detect less miRNA. Cells were gated and binned by mRuby3 (FIG. 8G) or mTagBFP2 (FIG. 8H), respectively. Dots and shaded regions were calculated as described in FIG. 2C. Unregulated represents either a mRuby3-divergent promoter-EGFP construct (FIG. 8G) or a mTagBFP2-divergent promoter-iRFP670 construct (FIG. 8H) without the regulation cassettes and the target sites. The new single transcript design shows some dosage-independence ranges, but still presents the miRNA-independent inhibition to some extent.

FIGS. 9A-9B depict non-limiting exemplary data related to biologically dead T6B failing to remove TNRC6-dependent inhibition of the miR-L 4×17 bp circuit. Flow cytometry was performed on the cells transfected with the miR-L 4×17 bp circuit (FIG. 9A) or miR-L 1×21 bp circuit (FIG. 9B) described in FIG. 2B along with the fluorescent protein-only negative control, the T6B peptide, or the catalytically dead T6B peptide (denoted as T6B^{Mut}), respectively. Cells were gated and binned by mRuby3 intensities. Dots and shaded regions were calculated as described in FIG. 2C. The design of the catalytically dead T6B peptide was consistent with the previous literature.

FIG. 10 depicts non-limiting exemplary data related to the dosage response curves of multiple miR-L 4×n circuits. Flow cytometry was performed on the cells transfected with the miR-L regulating 4×n circuits described in FIG. 3A. Gray rectangle indicates the gated region of the FIG. 3A left panel. Cells were gated and binned by mRuby3 intensities. Dots were calculated as described in FIG. 2C.

FIGS. 11A-11B depict non-limiting exemplary data related to the dosage response curves of the inducible DIMMER circuits in TRex cells. Flow cytometry was performed on the TRex cell line which was transfected with the 4×17 bp (FIG. 11A) or 4×18 bp (FIG. 11B) DIMMER construct, and the EGFP was driven by the CMV-2×TetO promoter. The concentrations of the 4-epi-Tetracycline, from purple to yellow, were 0, 10, 33.3, 100, 333.3 ng/mL. The gray curve denotes the mRuby-only transfection control, and the 4-epi-Tetracycline concentration is 333.3 ng/mL.

FIGS. 12A-12B depicts non-limiting exemplary data related to circuit designs—initial designs of synmiR-2 and synmiR-3 didn't work, and synmiR-6 worked but showed a sequence similarity to the endogenous miRNA. Flow cytometry was performed on the cells co-transfected with the circuit shown in FIG. 4A using the initial sequence of synmiR-2 (FIG. 12A, left panel), synmiR-3 (FIG. 12A, right panel), synmiR-6 (FIG. 12B), and their corresponding targets with a single, fully complementary target site, respectively. The BFP only control does not have the 3′UTR miRNA. Cells were gated by mRuby3 and mTagBFP2 intensities and binned by mTagBFP2 intensities. Relative expression levels were quantified as described in FIG. 4B. Dots and shaded regions were calculated as described in FIG. 2C.

FIG. 13 depicts non-limiting exemplary data related to 4×17 bp target of synmiR-4 and synmiR-5 showing strong regulation. The cells were transfected with the circuit architecture in FIG. 2A harboring either the synmiR-4 cassette and the corresponding 4×17 bp target, or synmiR-5 cassette and the corresponding 4×17 bp target and performed flow cytometry. Cells were gated and binned by mRuby3 intensities. Dots and shaded regions were calculated as described in FIG. 2C.

FIG. 14 depicts non-limiting exemplary data related to a gallery of all the IFFL designs based on different miRNAs and targets. The cells were transfected with the circuit architecture in FIG. 2B harboring the miRNA cassette denoted on the top of the plot and the corresponding target. Dots and shaded regions were calculated as described in FIG. 2C. Target site nomenclature was described in FIG. 4F.

FIGS. 15A-15B depicts non-limiting exemplary data related to the IFFL working across different cell lines. (FIG. 15A) The circuit described in FIG. 2B regulated by the miRNA and target site denoted was transfected into different cell lines and performed flow cytometry. Cells were gated and binned by mRuby3 intensities. Dots and shaded regions were calculated as described in FIG. 2C. Black vertical lines intersecting the x axis suggest the threshold levels of mRuby3 fluorescence intensities. Black horizontal lines intersecting the y axis suggest the saturating levels of EGFP fluorescence intensities. (FIG. 15B) The values of the intersection points of the gray lines with the axes in FIG. 15A were plotted as scatters. The black solid lines were calculated as described in FIG. 5R.

FIG. 16 depicts non-limiting exemplary data related to plotting of the normalized transcripts per million (TPM) of the synthetic miRNA expressing cells versus the mean TPM. The mean TPM was calculated by averaging all the TPM of all the microRNA-expressing cell samples. Solid line indicates where the TPM of the two samples were equal to each other. Dashed lines indicate where the TPM of the synmiR expressing cells is 10 fold of the mean, or where the TPM of the latter is 10 fold of the former.

FIGS. 17A-17C depicts non-limiting exemplary data related to IFFL improving fluorescence imaging. Flow cytometry was performed on cells that were transfected with the EGFR-mEGFP with or without the DIMMER module (FIG. 17A) and the dCas9-EGFP with or without the DIMMER module (FIG. 17B), respectively. Cells were gated and binned by mRuby3 intensities. Dots and shaded regions were calculated as described in FIG. 2C. (FIG. 17C) shows the quantification of the signal-to-noise ratio (SNR) of the dots in the cells transfected with the dCas9-EGFP with or without the DIMMER module. Each scatter represents one dot inside a cell.

FIG. 18 depicts a non-limiting exemplary schematic of reactions in model.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “vector” refers to a polynucleotide construct, typically a plasmid or a virus, used to transmit genetic material to a host cell (e.g., a target cell). Vectors can be, for example, viruses, plasmids, cosmids, or phage. A vector can be a viral vector. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “expression vector” refers to a vector that directs expression of an RNA or polypeptide (e.g., a synthetic protein circuit component) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, 2A sequences or elements refer to small peptides introduced as a linker between two proteins, allowing autonomous intraribosomal self-processing of polyproteins (See e.g., de Felipe. Genetic Vaccines and Ther. 2: 13 (2004); deFelipe et al. Traffic 5:616-626 (2004)). These short peptides allow co-expression of multiple proteins from a single vector. Many 2A elements are known in the art. Examples of 2A sequences that can be used in the methods and system disclosed herein, without limitation, include 2A sequences from the foot-and-mouth disease virus (F2A), equine rhinitis A virus (E2A), Thosea asigna virus (T2A), and porcine tescho virus-1 (P2A).

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “variant” refers to a polynucleotide (or polypeptide) having a sequence substantially similar to a reference polynucleotide (or polypeptide). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, the term “effective amount” refers to an amount sufficient to effect beneficial or desirable biological and/or clinical results.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may reduce the level of RAS signaling in the subject, thereby to reduce, alleviate, or eradicate the symptom(s) of the disease(s). As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, ammo acids, carbohydrates including glucose, mannose, or dextrins, chelating agents such as EDTA, sugar alcohols such as mannitol or sorbitol, salt-forming counterions such as sodium, and nonionic surfactants such as Tween, polyethylene glycol (PEG), and Pluronics. Auxiliary, stabilizer, emulsifier, lubricant, binder, pH adjuster controller, isotonic agent and other conventional additives may also be added to the carriers.

miRNA Circuits for Controlled Gene Expression

An important requirement in diverse areas of biomedical science and applications, including gene and cell therapy, is to express a protein of interest at a defined level in terms of number of RNA and/or protein copies per cell, or in terms of RNA and/or protein concentration per cell. This is often challenging due to uncontrollable factors, such as the number of copies of a transgene delivered to a cell, stochastic fluctuations (“noise”) in transcription and translation, molecular and functional heterogeneity among individual cells that impacts expression, and variation in the micro-environments of different cells, including variation in the levels of extrinsic signals.

Regulatory circuits that are compact, easy to incorporate into expression systems and delivery vectors, and can compensate for these uncontrolled factors to make expression levels more precise would be valuable in multiple applications. Provided herein include new regulatory circuits and design principles that limit gene expression variation, and reduce its sensitivity to gene copy number (gene dosage) and other factors. Provided herein include a variety of different circuit designs and implementations.

The problem of precise control appears in many contexts. In gene therapy, most vectors produce a distribution of numbers of gene copies in different cells. However, in many diseases that are candidates for gene therapy, therapeutic benefit depends on expressing the protein within a therapeutic window. Too little expression does not provide benefit, while too much expression can cause other diseases or be toxic. This occurs, for example, Rett syndrome, Angelman's syndrome, and spinal muscular atrophy, among others. Engineered cell therapies can similarly require defined expression levels of specific functional transgenes in cells. Overexpression of receptors, modulators, or effectors in the cell therapy can make it less effective or toxic. A third category involves metabolic engineering to create cells that can produce drugs, biologics, materials, or other components. In these systems, enzymes and other proteins need to be expressed at specific levels to maximize the amount and quality of production. A fourth category is in research applications. There, the ability to accurately assess the function of a wild-type protein, mutated protein (mutein), de novo designed protein, or the function of an RNA, including mRNA, miRNA, lncRNA, or any other class of RNA, whether modified from natural sequences or designed synthetically, can sensitively depend on the ability to achieve a precise expression level in the cell. Similarly, the ability to accurately determine the spatial pattern of protein localization within a cell through labeling of proteins or RNAs of interest with fluorescent proteins or other dyes, possibly fused to specific binders such as single-chain antibodies or nanobodies or other binders, can depend on limiting expression of the labeling proteins to a particular level. Otherwise, too much expression can lead to background fluorescence, obscuring the signal. These examples highlight specific use cases, but the problem of limiting expression of specific RNA, protein, and other components to particular expression windows applies in a broad range of contexts. The methods and compositions provided herein can enable precise control in such use cases.

Provided herein include, in some embodiments, a set of miRNA regulatory circuits constructed by expressing a gene of interest in one (“forward”) direction from a bidirectional promoter with an miRNA target site in its 3′ UTR while the corresponding mi RNA is generated inside an intron of the gene expressed in the reverse direction. The circuit architecture is an example of a broader class of circuit architectures termed incoherent-feedforward loops. In the correct operating regime, it can provide gene dosage compensation, such that increasing the number of copies of the gene in a cell increases miRNA expression enough to compensate for the increased transcription of the target gene, restricting target expression at a constant maximum level. Previous work has shown that synthetic IFFL circuits can successfully buffer gene expression to variations in gene dosage, noise from upstream regulators, competition for cellular resources, or general perturbations. In addition, synthetic miRNA incoherent feedforward loop circuits can allow gene and cell therapies to express their components within physiological ranges where unregulated expression would be harmful.

However, in all previous approaches, fully complementary miRNA target sites were used to implement dosage compensating circuits. These designs fail to fully compensate for changes in gene dosage as shown herein (See Example 1). Rather, circuits designed with a single-copy, fully complementary target tend to exhibit output expression that scales as the square root of gene dosage. The methods and compositions provided herein can overcome this issue through new circuit designs.

More specifically, in some embodiments of the disclosed methods and compositions, and without being bound by any particular theory, this issue is corrected by making two key changes to the design: First, the complementarity of the target sites was reduced. Second, the number of tandem binding sites in the target gene was increased. This combined change is not obvious because it depends on two features of miRNA regulation: first, the use of a “scaffold” protein known as TNRC6 or GW182; and, second, the fact that different levels of complementarity lead to different types of regulation. More specifically, full complementarity leads to “slicer” activity, which can produce target-mediated miRNA degradation (TDMD), which leads to an effective feedback that disrupts the ability of previous circuits to achieve full dosage compensation. Combining these two changes, together with additional optimization described in the attached manuscript, can achieve improved gene dosage compensation.

Also provided herein include methods to quantitatively tune the circuit's steady state output to enable the user to specify expression levels through sequence design. Specifically, as detailed in Example 1, provided herein are sequences with variable target site complementarity, appropriate numbers of miRNA generating cassettes inside of the synthetic intron, and variable strengths of the promoters expressing the miRNA. It is demonstrated how these three parameters can be modified together to achieve different dosage compensated expression levels. Additionally, provided herein include a means for generating orthogonal variants of the mi RNA circuits that can be used to simultaneously and independently regulate multiple genes in the same cell. A set of sequences is described, designated synmiRs, that can allow independent tuning of expression of multiple target genes. To generate these orthogonal variants reliably, a constrained-random generation system was created that reliably generates multiple fully orthogonal miRNA and target sequences. This allows one to use multiple circuits to independently regulate multiple genes, and maintain them at different set points, in the same cell or system. To demonstrate the usefulness of these circuits, it was shown in Example 1 how they can be used to increase the signal-to-noise ratio in imaging applications. It was also demonstrated that the system can enhance the control of gene expression from transiently expressed DNA as well as transgenes that are stably integrated in the genome at varying copy numbers. Overall, this technology dramatically improves upon previous miRNA-based gene expression control circuits. It expands the dynamic range of dosage-independent control, improving the agreement between the circuit output at low and high dosages. It allows the functionality more consistent and robust, and allows more flexibility in the design of miRNA circuits.

In some embodiments of the methods and compositions provided herein, the target gene(s) of interest (e.g., payload gene) in this circuit can be swapped out to allow regulation of expression of arbitrary genes, including both protein coding and non-coding (e.g. lncRNA) genes. For example, the target gene can be a therapeutic gene in the context of gene therapy including but not limited to MeCP2, SMN, UBE3A, or other genes which are used or anticipated to be used in gene therapies. In the context of cell therapy, the regulated gene could be composed of a synthetic Chimeric Antigen Receptor (CAR), T Cell Receptor (TCR), SynNotch receptor, or other synthetic receptor. In some embodiments, the methods and compositions provided herein can enable controlled regulation of cell signaling molecules including cytokines and chemokines in cell therapies or other contexts. Expression of these proteins is expected to enhance cell therapies, but overexpression can be deleterious.

In some embodiments, the methods and compositions provided herein can also be employed for therapeutic and non-therapeutic gene editing. Overexpression of gene editors can lead to off-target editing as well as non-specific toxicity. In this context, regulated target genes could include any of the many CRISPR-related proteins. These include but are not limited to Cas9, Cas12, and Cas13; CRISPR base editing nickase or dcas9-APOBEC1; multiplex gene editors such as Cas12; CRISPR transcriptional modulators such as dcas9-KRAB or dcas9-VP64, as well as epigenetic regulators with different repression, activation, or other effector domains; catalytically inactive “dead” variants of Cas9 and Cas13 used for imaging and other applications, such as, for example, CRISPR imaging with Cas9 or Cas13 or general protein-fluorescent protein fusions. In some embodiments, the methods and compositions provided herein can also be employed to regulate non-CRISPR gene editors, e.g., those based on TALE-based CRISPR free base editors could also be regulated using these systems.

In some embodiments, the methods and compositions provided herein can also be employed for regulating the expression of proteins used in RNA export systems, and virus-like particle (VLP) production in cells to avoid overexpression toxicity. More generally, the regulated gene may be any transcription factor, protease, kinase, phosphatase, or other regulatory protein involved in constructing a synthetic biological circuit. In the context of regenerative medicine, the methods and compositions provided herein can enable expression of ectopic transcription factors at defined levels that optimally reprogram cell states to achieve desired cell types more efficiently and selectively. In each of the application categories discussed above, the circuit can play a key role in assist in expressing the protein at a non-toxic physiological level in the presence of noise, reducing off-target effects from overexpression, and reducing background activity to enhance signal to background ratio.

The methods and compositions provided herein have been demonstrated in mammalian cells, and in some embodiments, the methods and compositions provided herein can function in cells from other species that contain the core machinery necessary for miRNA regulation. The miRNA-based gene regulation circuits disclosed here broadly enable precise control of gene expression in numerous contexts, including both basic research and applications, such as gene therapy. They introduce new designs and mechanisms that improve dosage-invariance as well as other properties compared to currently available technologies.

Synthetic biology allows for rational design of circuits that confer new functions in living cells. For example, CHOMP (circuits of hacked orthogonal modular proteases) enables design of composable protein circuit components. Many natural cellular functions can be implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety. The systems, methods, compositions, and kits provided herein can, in some embodiments, be employed in concert with the systems, methods, compositions, and kits for described in U.S. Patent Publication No. 2023/0076395, entitled, “CELL-TO-CELL DELIVERY OF RNA CIRCUITS,” and in U.S. Patent Publication No. 2023/0071834, entitled, “EXPORTED RNA REPORTERS FOR LIVE-CELL MEASUREMENT,” the contents of which are incorporated herein by reference in their entirety. The systems, methods, compositions, and kits provided herein can, in some embodiments, be employed in concert with the systems, methods, compositions, and kits described in PCT Application No. PCT/US23/69663, entitled, “A SYNTHETIC PROTEIN-LEVEL NEURAL NETWORK IN MAMMALIAN CELLS,” filed Jul. 5, 2023, the content of which is incorporated herein by reference in its entirety. The systems, methods, compositions, and kits provided herein can, in some embodiments, be employed in concert with the systems, methods, compositions, and kits described in PCT Application Publication No. WO2020117713A1, entitled, “In situ readout of dna barcodes,” the content of which is incorporated herein by reference in its entirety. The systems, methods, compositions, and kits provided herein can, in some embodiments, be employed in concert with the systems, methods, compositions, and kits described in U.S. patent application Ser. Nos. 17/820,232, 17/820,235, and 18/757,460, the contents of which are incorporated herein by reference in their entireties. The systems, methods, compositions, and kits provided herein can, in some embodiments, be employed in concert with the systems, methods, compositions, and kits described in PCT Application Publication No. WO2024081912A1, entitled, “PROTEIN-BASED SIGNAL AMPLIFICATION,” the content of which is incorporated herein by reference in its entirety. The systems, methods, compositions, and kits provided herein can, in some embodiments, be employed in concert with the systems, methods, compositions, and kits described in U.S. patent application Ser. No. 18/799,870, entitled, “MOLECULAR RECORDING METHODS AND SYSTEMS TO CAPTURE LINEAGE RELATIONSHIPS IN DIFFERENTIATING STEM CELLS,” filed Aug. 9, 2024, the content of which is incorporated herein by reference in its entirety. The systems, methods, compositions, and kits provided herein can, in some embodiments, be employed in concert with the systems, methods, compositions, and kits described in Du, Rongrong, et al. (“miRNA circuit modules for precise, tunable control of gene expression.” BioRxiv (2024)), the content of which, including any supporting or supplemental information or material, is incorporated herein by reference in its entirety.

Disclosed herein include compositions (e.g., nucleic acid compositions, one or more cells). Disclosed herein include nucleic acid compositions. In some embodiments, the nucleic acid composition comprises: a first promoter sequence operably linked to a first polynucleotide comprising one or more miRNA cassettes. In some embodiments, the first promoter sequence is capable of inducing transcription of the first polynucleotide to generate a first transcript. In some embodiments, the first transcript is capable of being processed to generate said miRNA. In some embodiments, the nucleic acid composition comprises: a second promoter sequence operably linked to a second polynucleotide comprising a payload gene. In some embodiments, the payload gene comprises a miRNA target region comprising one or more miRNA target sequences. In some embodiments, the second promoter sequence is capable of inducing transcription of the second polynucleotide to generate a payload transcript. In some embodiments, the payload transcript is capable of being translated to generate a payload protein. In some embodiments, the first promoter sequence and the second promoter sequence are components of a bidirectional promoter. In some embodiments, the first promoter sequence and the second promoter sequence are in reverse complementary orientation with respect to each other in the bidirectional promoter.

Disclosed herein include compositions comprising one or more cells. In some embodiments, the one or more cells comprise: a first promoter sequence operably linked to a first polynucleotide comprising one or more miRNA cassettes. In some embodiments, the first promoter sequence is capable of inducing transcription of the first polynucleotide to generate a first transcript. In some embodiments, the first transcript is capable of being processed to generate said miRNA. In some embodiments, the one or more cells comprise: a second promoter sequence operably linked to a second polynucleotide comprising a payload gene. In some embodiments, the payload gene comprises a miRNA target region comprising one or more miRNA target sequences. In some embodiments, the second promoter sequence is capable of inducing transcription of the second polynucleotide to generate a payload transcript. In some embodiments, the payload transcript is capable of being translated to generate a payload protein. In some embodiments, the first promoter sequence and the second promoter sequence are components of a bidirectional promoter. In some embodiments, the first promoter sequence and the second promoter sequence are in reverse complementary orientation with respect to each other in the bidirectional promoter.

In some embodiments, the miRNA is capable of binding the one or more miRNA target sequences, thereby reducing the stability of the payload transcript and/or reducing the translation of the payload transcript. In some embodiments, the first polynucleotide comprises a dosage gene. In some embodiments, the first transcript is capable of being translated to generate dosage indicator protein. In some embodiments, an intron is located in the dosage gene 3′UTR, dosage gene 5′UTR, or between dosage gene exons, optionally a synthetic intron. In some embodiments, the intron comprises the one or more miRNA cassettes. In some embodiments, the intron comprises one or more of: (i) an intronic insert encoding a miRNA, (ii) a donor splice site, (iii) an acceptor splice site, (iv) a branch point domain; and (v) a polypyrimidine tract. In some embodiments, the miRNA, or precursor thereof, is capable of being released from said intron by an intron excision mechanism selected from the group comprising cellular RNA splicing and/or processing machinery, nonsense-mediated decay (NMD) processing, or any combination thereof, optionally the precursor comprises a pri-miRNA. In some embodiments, the dosage indicator protein is detectable, optionally the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

In some embodiments, the one or more cells comprise two or more cells. In some embodiments, the two or more cells comprise a first cell and a second cell. In some embodiments, the first cell is a first cell type and/or the second cell is a second cell type. In some embodiments, the (i) first and second cell type and/or (ii) the one or more cells, are selected from the group comprising: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

In some embodiments, the first polynucleotide is transcribed at a rate at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) higher in the first cell as compared to the second cell. In some embodiments, the first polynucleotide is translated at a rate at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) higher in the first cell as compared to the second cell. In some embodiments, the first polynucleotide is present at a copy number at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) higher in the first cell as compared to the second cell. In some embodiments, the threshold dosage at which payload protein expression is saturated is at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) higher in the first cell as compared to the second cell. In some embodiments, the rate of transcription of the second polynucleotide and/or the rate of translation of the payload transcript varies between a first time point and a second time point in a single cell and/or varies between the first cell and the second cell at the same time point. In some embodiments, in the absence of the miRNA, the payload protein reaches untuned steady state payload protein levels in the one or more cells. In some embodiments, untuned steady state payload protein levels range between a lower untuned threshold and an upper untuned threshold of an untuned expression range. In some embodiments, steady state dosage indicator protein levels reflect untuned steady state payload protein levels. In some embodiments, in the presence the miRNA, the payload protein reaches tuned steady state payload protein levels in the one or more cells.

In some embodiments, tuned steady state payload protein levels range between a lower tuned threshold and an upper tuned threshold of a tuned expression range, optionally wherein upper tuned threshold of the tuned expression range is a saturating expression level. In some embodiments, at the first time point and the second time point in a single cell, the steady state levels of the payload protein remain within the tuned expression range. In some embodiments, in the first cell and the second cell at the same time point, the steady state levels of the payload protein remain within the tuned expression range. In some embodiments, the lower tuned threshold and/or the upper tuned threshold of a tuned expression range is capable of being configured by modulating one or more of (i) the number of miRNA cassettes within the first polynucleotide, (ii) the number of miRNA target sequences in the miRNA target region, (iii) the complementarity between the miRNA and the one or more miRNA target sequences, and (iv) strength of the first promoter sequence and/or second promoter sequence. In some embodiments, the difference between the lower untuned threshold and the upper untuned threshold of the untuned expression range is greater than about two orders of magnitude. In some embodiments, the difference between the lower tuned threshold and the upper tuned threshold of the tuned expression range is less than about one order of magnitude. In some embodiments, the payload protein is efficacious at steady state payload protein levels within the tuned expression range. In some embodiments, the payload protein is inefficacious and/or toxic at steady state payload protein levels above and/or below the tuned expression range. In some embodiments, the payload protein is capable of inducing an immunogenic response and/or a cytokine storm at steady state payload protein levels outside the tuned expression range. In some embodiments, tuned steady state payload protein levels comprise a therapeutic level of the payload protein. In some embodiments, the steady state payload protein levels remain within the tuned expression range across multiple cell types, titers of viral vector, and/or viral vector capsid types. In some embodiments, the tuned steady state payload protein levels are robust to tissue tropism and stochastic expression.

In some embodiments, the miRNA comprises a nucleotide sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NOs: 75-87, portions thereof, and/or complementary (e.g., reverse complementary) sequences thereof. In some embodiments, the miRNA cassette comprises a nucleotide sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NOs: 1-13, portions thereof, and/or complementary (e.g., reverse complementary) sequences thereof. In some embodiments, the miRNA target region comprises a nucleotide sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to SEQ ID NOs: 14-74, portions thereof, and/or complementary (e.g., reverse complementary) sequences thereof.

In some embodiments, the miRNA is about 20, 21, 22, 23, or 24, nucleotides (nt) in length. In some embodiments, the sequence of the miRNA is orthogonal to the one or more cells, optionally a miRNA sequence targeting Renilla luciferase. In some embodiments, the miRNA target region is situated in the 3′ UTR, 5′ UTR, or coding region of the payload gene. In some embodiments, the miRNA target region comprises at least about 1, 2, 3, 4, 5, 6, 7, 8, 9, 10, 11, 12, 13, 14, 15, 16, 17, 18, 19, or 20 miRNA target sequences. In some embodiments, a payload transcript is capable of being simultaneously bound by multiple miRNA-loaded Argonaute (Ago) complexes, optionally via TNRC6. In some embodiments, 5, 6, 7, 8, 9, 10, 11, 12, 13, 14, 15, 16, 17, 18, 19, 20, 21, nucleotides (nt) of a miRNA target sequence is complementary to the miRNA, optionally 1, 2, 3, 4, 5, 6, 7, 8, 9, 10, 11, 12, 13, 14, or 15 nt of the 3′ end of the miRNA lack complementarity. In some embodiments, the complementarity between the miRNA and a miRNA target sequence is at least, or at most, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or a number or a range between any two of these values. In some embodiments, the miRNA comprises 5-8 GC nucleotides, optionally 1-4 in the seed region and 1-2 in the extensive region. In some embodiments, the composition comprises one or more supplemental payload genes and one or more supplemental miRNA, wherein the supplemental miRNA differ in sequence with respect to each other.

TABLE 1
miRNA and targets

| miRNA/target | | Usage in the |
|---|---|---|
| name | Sequence | figures |
| synmiR-1 | TTGAATGAGGCTTCAGTACTTTACAGAATCGTTGCCTGCACATCTT | 4D, 4E, 4F, 14, |
| | GGAAACACTTGCTGGGATTACTTCGACTTCTTAACCCAACAGAAG | 16 |
| | GCTCGAGAAGGTATATTGCTGTTGACAGTGAGCGCCAACATAAG | |
| | CATAAACTACGATAGTGAAGCCACAGATGTATCGTAGTTTATGC | |
| | TTATGTTGATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 1) | |
| synmiR- | TTGAATGAGGCTTCAGTACTTTACAGAATCGTTGCCTGCACATCTT | 12A |
| 2_initial | GGAAACACTTGCTGGGATTACTTCGACTTCTTAACCCAACAGAAG | |
| version | GCTCGAGAAGGTATATTGCTGTTGACAGTGAGCGCTAATCTACA | |
| | AAGTGACATAAATAGTGAAGCCACAGATGTATTTATGTCACTTT | |
| | GTAGATTAATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 2) | |
| synmiR- | TTGAATGAGGCTTCAGTACTTTACAGAATCGTTGCCTGCACATCTT | 4D, 4E, 4F, 4H, |
| 2_20G | GGAAACACTTGCTGGGATTACTTCGACTTCTTAACCCAACAGAAG | 14, 16 |
| | GCTCGAGAAGGTATATTGCTGTTGACAGTGAGCGCTGATCTACA | |
| | AAGTGACATAAATAGTGAAGCCACAGATGTATTTATGTCACTTT | |
| | GTAGATCAATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 3) | |
| synmiR- | TTGAATGAGGCTTCAGTACTTTACAGAATCGTTGCCTGCACATCTT | 12A |
| 3_initial | GGAAACACTTGCTGGGATTACTTCGACTTCTTAACCCAACAGAAG | |
| version | GCTCGAGAAGGTATATTGCTGTTGACAGTGAGCGCGAAAAGTTT | |
| | ATAATATCTTGATAGTGAAGCCACAGATGTATCAAGATATTATA | |
| | AACTTTTCATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 4) | |
| synmiR- | TTGAATGAGGCTTCAGTACTTTACAGAATCGTTGCCTGCACATCTT | 4D, 4E, 4F, 5E, |
| 3_19G | GGAAACACTTGCTGGGATTACTTCGACTTCTTAACCCAACAGAAG | 5F, 14, 16 |
| | GCTCGAGAAGGTATATTGCTGTTGACAGTGAGCGCGAGAAGTTT | |
| | ATAATATCTTGATAGTGAAGCCACAGATGTATCAAGATATTATA | |
| | AACTTCTCATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 5) | |
| synmiR-4 | TTGAATGAGGCTTCAGTACTTTACAGAATCGTTGCCTGCACATCTT | 4D, 4E, 4F, |
| | GGAAACACTTGCTGGGATTACTTCGACTTCTTAACCCAACAGAAG | 13-16 |
| | GCTCGAGAAGGTATATTGCTGTTGACAGTGAGCGCCGTAAAGAT | |
| | CATGAAATTGAATAGTGAAGCCACAGATGTATTCAATTTCATGA | |
| | TCTTTACGATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 6) | |
| synmiR-5 | TTGAATGAGGCTTCAGTACTTTACAGAATCGTTGCCTGCACATCTT | 4D, 4E, 4F, 4H, |
| | GGAAACACTTGCTGGGATTACTTCGACTTCTTAACCCAACAGAAG | 13-16 |
| | GCTCGAGAAGGTATATTGCTGTTGACAGTGAGCGCAGCATCTAT | |
| | CTAACGGTTTGATAGTGAAGCCACAGATGTATCAAACCGTTAG | |
| | ATAGATGCTATGCCTACTGCCTCGGACTTCAAGGGGCTAGAATTC | |
| | GAGCAATTATCTTGTTTACTAAAACTGAATACCTTGCTATCTCTTT | |
| | GATACATTTTTACAAAGCTGAATTAAAATGGTATAAATTAAATCA | |
| | CTTTTCCTGACCATTCATCCTCTTTCTTTTTCCT (SEQ ID NO: 7) | |
| synmiR-6 | TTGAATGAGGCTTCAGTACTTTACAGAATCGTTGCCTGCACATCTT | 12B |
| | GGAAACACTTGCTGGGATTACTTCGACTTCTTAACCCAACAGAAG | |
| | GCTCGAGAAGGTATATTGCTGTTGACAGTGAGCGCCTCTTATCAA | |
| | GCAGTTTCATATAGTGAAGCCACAGATGTATATGAAACTGCTTG | |
| | ATAAGAGATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 8) | |
| synmiR-7 | TTGAATGAGGCTTCAGTACTTTACAGAATCGTTGCCTGCACATCTT | 4D, 4E, 4F, 14, |
| | GGAAACACTTGCTGGGATTACTTCGACTTCTTAACCCAACAGAAG | 16 |
| | GCTCGAGAAGGTATATTGCTGTTGACAGTGAGCGCAGAAATGGT | |
| | GTAATTTAGCAATAGTGAAGCCACAGATGTATTGCTAAATTACA | |
| | CCATTTCTATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 9) | |
| synmiR-8 | TTGAATGAGGCTTCAGTACTTTACAGAATCGTTGCCTGCACATCTT | 4D, 4E, 4F, 14, |
| | GGAAACACTTGCTGGGATTACTTCGACTTCTTAACCCAACAGAAG | 16 |
| | GCTCGAGAAGGTATATTGCTGTTGACAGTGAGCGCCGTTGAGAT | |
| | TTAAAGATCGAATAGTGAAGCCACAGATGTATTCGATCTTTAAA | |
| | TCTCAACGATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 10) | |
| synmiR-9 | TTGAATGAGGCTTCAGTACTTTACAGAATCGTTGCCTGCACATCTT | 4D, 4E, 4F, 14, |
| | GGAAACACTTGCTGGGATTACTTCGACTTCTTAACCCAACAGAAG | 16 |
| | GCTCGAGAAGGTATATTGCTGTTGACAGTGAGCGCCAACACCTT | |
| | TCTTAAAACTTATAGTGAAGCCACAGATGTATAAGTTTTAAGAA | |
| | AGGTGTTGATGCCTACTGCCTCGGACTTCAAGGGGCTAGAATTC | |
| | GAGCAATTATCTTGTTTACTAAAACTGAATACCTTGCTATCTCTTT | |
| | GATACATTTTTACAAAGCTGAATTAAAATGGTATAAATTAAATCA | |
| | CTTTTCCTGACCATTCATCCTCTTTCTTTTTCCT (SEQ ID NO: 11) | |
| synmiR-10 | TTGAATGAGGCTTCAGTACTTTACAGAATCGTTGCCTGCACATCTT | 4D, 4E, 4F, 14, |
| | GGAAACACTTGCTGGGATTACTTCGACTTCTTAACCCAACAGAAG | 16 |
| | GCTCGAGAAGGTATATTGCTGTTGACAGTGAGCGCGGTAATCTT | |
| | AAGCATAGATGATAGTGAAGCCACAGATGTATCATCTATGCTTA | |
| | AGATTACCATGCCTACTGCCTCGGACTTCAAGGGGCTAGAATTCG | |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 12) | |
| miR-L | TTGAATGAGGCTTCAGTACTTTACAGAATCGTTGCCTGCACATCTT | 2C-F, 2H-I, |
| | GGAAACACTTGCTGGGATTACTTCGACTTCTTAACCCAACAGAAG | 3A-E, 4B, 4D, 4E, |
| | GCTCGAGAAGGTATATTGCTGTTGACAGTGAGCGCAGGAATTAT | 4H, 5A, 5B |
| | AATGCTTATCTATAGTGAAGCCACAGATGTATAGATAAGCATTA | 5D-F, 6B, 6D-F, |
| | TAATTCCTATGCCTACTGCCTCGGACTTCAAGGGGCTAGAATTCG | 6H, 8-11, 15-17 |
| | AGCAATTATCTTGTTTACTAAAACTGAATACCTTGCTATCTCTTTG | |
| | ATACATTTTTACAAAGCTGAATTAAAATGGTATAAATTAAATCAC | |
| | TTTTCCTGACCATTCATCCTCTTTCTTTTTCCT (SEQ ID NO: 13) | |

Note: the mature miRNA sequence (including the sense chain and the antisense chain) is bolded

| | | |
|---|---|---|
| miR- | AGGAATTATAATGCTTATCTA (SEQ ID NO: 14) | 2C, 2D, 2I, 4B, |
| L_1 × 21 bp | | 4D, 4E, 9B |
| miR- | TACTATTATAATGCTTATCTA (SEQ ID NO: 15) | 2D |
| L_1 × 17 bp | | |
| miR- | CTTAATTATAATGCTTATCTA (SEQ ID NO: 16) | 2D |
| L_1 × 18 bp | | |
| miR- | TTGAATTATAATGCTTATCTA (SEQ ID NO: 17) | 2D |
| L_1 × 19 bp | | |
| miR- | TGGAATTATAATGCTTATCTA (SEQ ID NO: 18) | 2D |
| L_1 × 20 bp | | |
| miR- | TACTATTATAATGCTTATCTATACTATTATAATGCTTATCTA | 2E |
| L_2 × 17 bp | (SEQ ID NO: 19) | |
| miR- | TACTATTATAATGCTTATCTATACTATTATAATGCTTATCTATA | 2E |
| L_3 × 17 bp | CTATTATAATGCTTATCTA (SEQ ID NO: 20) | |
| miR- | TACTATTATAATGCTTATCTATACTATTATAATGCTTATCTATA | 2E, 2F, 2H, |
| L_4 × 17 bp | CTATTATAATGCTTATCTATACTATTATAATGCTTATCTA (SEQ | 3A-D, 4H, 5A, 5B, |
| | ID NO: 21) | 5D, 6E, 6F, 6H, |
| | | 8, 9A, 11A, |
| | | 15-17 |
| miR-L_4 × 8 bp | TACTTGGCATTATCTTATCTATACAACAATTTACTTGGCATTATCT | 3A |
| | TATCTATAATCACTTGTACTTGGCATTATCTTATCTATACAACTG | |
| | GATACTTGGCATTATCTTATCTA (SEQ ID NO: 22) | |
| miR-L_4 × 9 bp | TACTTGGCATTAGCTTATCTATACAACAATTTACTTGGCATTAGC | 3A, 10 |
| | TTATCTATAATCACTTGTACTTGGCATTAGCTTATCTATACAACT | |
| | GGATACTTGGCATTAGCTTATCTA (SEQ ID NO: 23) | |
| miR- | TACTTGGCATTTGCTTATCTATACAACAATTTACTTGGCATTTGC | 3A |
| L_4 × 10 bp | TTATCTATAATCACTTGTACTTGGCATTTGCTTATCTATACAACT | |
| | GGATACTTGGCATTTGCTTATCTA (SEQ ID NO: 24) | |
| miR- | TACTTGGCATATGCTTATCTATACAACAATTTACTTGGCATATGC | 3A |
| L_4 × 11 bp | TTATCTATAATCACTTGTACTTGGCATATGCTTATCTATACAACT | |
| | GGATACTTGGCATATGCTTATCTA (SEQ ID NO: 25) | |
| miR- | TACTTGGCAAATGCTTATCTATACAACAATTTACTTGGCAAATGC | 3A, 10 |
| L_4 × 12 bp | TTATCTATAATCACTTGTACTTGGCAAATGCTTATCTATACAACT | |
| | GGATACTTGGCAAATGCTTATCTA (SEQ ID NO: 26) | |
| miR- | TACTTGGCTAATGCTTATCTATACAACAATTTACTTGGCTAATGC | 3A, 10 |
| L_4 × 13 bp | TTATCTATAATCACTTGTACTTGGCTAATGCTTATCTATACAACT | |
| | GGATACTTGGCTAATGCTTATCTA (SEQ ID NO: 27) | |
| miR- | TACTTGGATAATGCTTATCTATACTTGGATAATGCTTATCTATA | 3A, 10 |
| L_4 × 14 bp | CTTGGATAATGCTTATCTATACTTGGATAATGCTTATCTA (SEQ | |
| | ID NO: 28) | |
| miR- | TACTTGTATAATGCTTATCTATACAACAATTTACTTGTATAATGC | 3A, 10 |
| L_4 × 15 bp | TTATCTATAATCACTTGTACTTGTATAATGCTTATCTATACAACT | |
| | GGATACTTGTATAATGCTTATCTA (SEQ ID NO: 29) | |
| miR- | TTATAATGCTTATCTAAATATTATAATGCTTATCTAAATATTAT | 3A, 10 |
| L_4 × 16 bp | AATGCTTATCTAAATATTATAATGCTTATCTA (SEQ ID NO: 30) | |
| miR- | AATTATAATGCTTATCTAAATAAATTATAATGCTTATCTAAATA | 3A, 3B, 6B, 6E, |
| L_4 × 18 bp | AATTATAATGCTTATCTAAATAAATTATAATGCTTATCTA (SEQ | 6F, 11B, 17A |
| | ID NO: 31) | |
| miR- | GAATTATAATGCTTATCTAAATAGAATTATAATGCTTATCTAA | 3A, 3B, 3E, 4H, |
| L_4 × 19 bp | ATAGAATTATAATGCTTATCTAAATAGAATTATAATGCTTATCT | 6E, 6F, 6H, |
| | A (SEQ ID NO: 32) | 17B-17C |
| miR- | TGGAATTATAATGCTTATCTATACAACAATTTGGAATTATAATG | 3A |
| L_4 × 20 bp | CTTATCTATAATCACTTGTGGAATTATAATGCTTATCTATACAA | |
| | CTGGATGGAATTATAATGCTTATCTA (SEQ ID NO: 33) | |
| miR- | AGGAATTATAATGCTTATCTAAGGAATTATAATGCTTATCTAA | 3A, 10 |
| L_4 × 21 bp | GGAATTATAATGCTTATCTAAGGAATTATAATGCTTATCTA | |
| | (SEQ ID NO: 34) | |
| miR-L_8 × 8 bp | TACTTGGCATTATCTTATCTATACAACAATTTACTTGGCATTATCT | 15 |
| | TATCTATAATCACTTGTACTTGGCATTATCTTATCTATACAACTG | |
| | GATACTTGGCATTATCTTATCTATAACTAGTTCTTTGATACTTGGC | |
| | ATTATCTTATCTATACAACAATTTACTTGGCATTATCTTATCTAT | |
| | AATCACTTGTACTTGGCATTATCTTATCTATACAACTGGATACTT | |
| | GGCATTATCTTATCTA (SEQ ID NO: 35) | |
| miR- | AGGAATTATAATGCTTATCTAAGGAATTATAATGCTTATCTAA | 8H |
| L_10 × 21bp | GGAATTATAATGCTTATCTAAGGAATTATAATGCTTATCTAAG | |
| | GAATTATAATGCTTATCTAAGGAATTATAATGCTTATCTAAGG | |
| | AATTATAATGCTTATCTAAGGAATTATAATGCTTATCTAAGGA | |
| | ATTATAATGCTTATCTAAGGAATTATAATGCTTATCTA (SEQ ID | |
| | NO: 36) | |
| synmiR- | CAACATAAGCATAAACTACGA (SEQ ID NO: 37) | 4D, 4E |
| 1_1 × 21 bp | | |
| synmiR- | TAATCTACAAAGTGACATAAA (SEQ ID NO: 38) | 12A |
| 2_1 × 21 bp_ | | |
| initial | | |
| synmiR- | TGATCTACAAAGTGACATAAA (SEQ ID NO: 39) | 4D, 4E |
| 2_1 × 21 bp_ | | |
| 20G | | |
| synmiR- | GAAAAGTTTATAATATCTTGA (SEQ ID NO: 40) | 12A |
| 3_1 × 21 bp_ | | |
| initial | | |
| synmiR- | GAGAAGTTTATAATATCTTGA (SEQ ID NO: 41) | 4D, 4E |
| 3_1 × 21 bp_ | | |
| 19G | | |
| synmiR- | CGTAAAGATCATGAAATTGAA (SEQ ID NO: 42) | 4D, 4E |
| 4_1 × 21 bp | | |
| synmiR- | AGCATCTATCTAACGGTTTGA (SEQ ID NO: 43) | 4D, 4E |
| 5_1 × 21 bp | | |
| synmiR- | CTCTTATCAAGCAGTTTCATA (SEQ ID NO: 44) | 12B |
| 6_1 × 21 bp | | |
| synmiR- | AGAAATGGTGTAATTTAGCAA (SEQ ID NO: 45) | 4D, 4E |
| 7_1 × 21 bp | | |
| synmiR- | CGTTGAGATTTAAAGATCGAA (SEQ ID NO: 46) | 4D, 4E |
| 8_1 × 21 bp | | |
| synmiR- | CAACACCTTTCTTAAAACTTA (SEQ ID NO: 47) | 4D, 4E |
| 9_1 × 21 bp | | |
| synmiR- | GGTAATCTTAAGCATAGATGA (SEQ ID NO: 48) | 4D, 4E |
| 10_1 × 21 bp | | |
| synmiR- | ACAATATTCGTATAACTACGATACAACAATTACAATATTCGTATA | 4F, 14 |
| 1_4 × 8 bp | ACTACGATAATCACTTGAAAATATTCGTATAACTACGATACAAC | |
| | TGGAACAATATTCGTATAACTACGA (SEQ ID NO: 49) | |
| synmiR- | ACAATATTCGTAAAACTACGATACAACAATTACAATATTCGTAAA | 14 |
| 1_4 × 9 bp | ACTACGATAATCACTTGAAAATATTCGTAAAACTACGATACAAC | |
| | TGGAACAATATTCGTAAAACTACGA (SEQ ID NO: 50) | |
| synmiR- | CATGAAGTTTTCAGACATAAATACAACAATTCATGAAGTTTTCAG | 14 |
| 2_4 × 8 bp | ACATAAATAATCACTTGCATGAAGTTTTCAGACATAAATACAACT | |
| | GGACATGAAGTTTTCAGACATAAA (SEQ ID NO: 51) | |
| synmiR- | CATGAAGTTTTCTGACATAAATACAACAATTCATGAAGTTTTCTG | 14 |
| 2_4 × 9 bp | ACATAAATAATCACTTGCATGAAGTTTTCTGACATAAATACAACT | |
| | GGACATGAAGTTTTCTGACATAAA (SEQ ID NO: 52) | |
| synmiR- | CATGAAGTTTTCAGACATAAATACAACAATTCATGAAGTTTTCAG | 4F, 4H, 14 |
| 2_8 × 8 bp | ACATAAATAATCACTTGCATGAAGTTTTCAGACATAAATACAACT | |
| | GGACATGAAGTTTTCAGACATAAATAACTAGTTCTTTGACATGAA | |
| | GTTTTCAGACATAAATACAACAATTCATGAAGTTTTCAGACATAA | |
| | ATAATCACTTGCATGAAGTTTTCAGACATAAATACAACTGGACAT | |
| | GAAGTTTTCAGACATAAA (SEQ ID NO: 53) | |
| synmiR- | TCTTTGTTTATAATATCTTGATACAACAATTTCTTTGTTTATAAT | 14 |
| 3_4 × 16 bp | ATCTTGATAATCACTTGTCTTTGTTTATAATATCTTGATACAACT | |
| | GGATCTTTGTTTATAATATCTTGA (SEQ ID NO: 54) | |
| synmiR- | TCTTTGTTTATAATATCTTGATACAACAATTTCTTTGTTTATAAT | 4F, 14 |
| 3_8 × 16 bp | ATCTTGATAATCACTTGTCTTTGTTTATAATATCTTGATACAACT | |
| | GGATCTTTGTTTATAATATCTTGATAACTAGTTCTTTGATCTTTG | |
| | TTTATAATATCTTGATACAACAATTTCTTTGTTTATAATATCTTG | |
| | ATAATCACTTGTCTTTGTTTATAATATCTTGATACAACTGGATCT | |
| | TTGTTTATAATATCTTGA (SEQ ID NO: 55) | |
| synmiR- | GTACTCTGATCATAAATTGAATACAACAATTGTACTCTGATCATA | 14 |
| 4_4 × 8 bp | AATTGAATAATCACTTGGTACTCTGATCATAAATTGAATACAACT | |
| | GGAGTACTCTGATCATAAATTGAA (SEQ ID NO: 56) | |
| synmiR- | GTACTCTGATCATAAATTGAATACAACAATTGTACTCTGATCATA | 4F, 14 |
| 4_8 × 8 bp | AATTGAATAATCACTTGGTACTCTGATCATAAATTGAATACAACT | |
| | GGAGTACTCTGATCATAAATTGAATAACTAGTTCTTTGAGTACTC | |
| | TGATCATAAATTGAATACAACAATTGTACTCTGATCATAAATTGA | |
| | ATAATCACTTGGTACTCTGATCATAAATTGAATACAACTGGAGTA | |
| | CTCTGATCATAAATTGAA (SEQ ID NO: 57) | |
| synmiR- | GTACTCTGATCAGAAATTGAATACAACAATTGTACTCTGATCAGA | S8, 15 |
| 4_8 × 9 bp | AATTGAATAATCACTTGGTACTCTGATCAGAAATTGAATACAACT | |
| | GGAGTACTCTGATCAGAAATTGAATAACTAGTTCTTTGAGTACTC | |
| | TGATCAGAAATTGAATACAACAATTGTACTCTGATCAGAAATTG | |
| | AATAATCACTTGGTACTCTGATCAGAAATTGAATACAACTGGAGT | |
| | ACTCTGATCAGAAATTGAA (SEQ ID NO: 58) | |
| synmiR- | GTACAAGATCATGAAATTGAATACAACAATTGTACAAGATCATG | 13 |
| 4_4 × 17 bp | AAATTGAATAATCACTTGGTACAAGATCATGAAATTGAATACAA | |
| | CTGGAGTACAAGATCATGAAATTGAA (SEQ ID NO: 59) | |
| synmiR- | CCATGAGTAATCTCGGTTTGATACAACAATTCCATGAGTAATCTC | 4H, 14 |
| 5_4 × 8 bp | GGTTTGATAATCACTTGCCATGAGTAATCTCGGTTTGATACAAC | |
| | TGGACCATGAGTAATCTCGGTTTGA (SEQ ID NO: 60) | |
| synmiR- | CCATGAGTAATCACGGTTTGATACAACAATTCCATGAGTAATCA | 14 |
| 5_4 × 9 bp | CGGTTTGATAATCACTTGCCATGAGTAATCACGGTTTGATACAA | |
| | CTGGACCATGAGTAATCACGGTTTGA (SEQ ID NO: 61) | |
| synmiR- | CCATGAGTACTAACGGTTTGATACAACAATTCCATGAGTACTAA | 14 |
| 5_4 × 12 bp | CGGTTTGATAATCACTTGCCATGAGTACTAACGGTTTGATACAA | |
| | CTGGACCATGAGTACTAACGGTTTGA (SEQ ID NO: 62) | |
| synmiR- | CCATGAGTAATCTCGGTTTGATACAACAATTCCATGAGTAATCTC | 14, 15 |
| 5_8 × 8 bp | GGTTTGATAATCACTTGCCATGAGTAATCTCGGTTTGATACAAC | |
| | TGGACCATGAGTAATCTCGGTTTGATAACTAGTTCTTTGACCATG | |
| | AGTAATCTCGGTTTGATACAACAATTCCATGAGTAATCTCGGTT | |
| | TGATAATCACTTGCCATGAGTAATCTCGGTTTGATACAACTGGAC | |
| | CATGAGTAATCTCGGTTTGA (SEQ ID NO: 63) | |
| synmiR- | CCATTCTATCTAACGGTTTGATACAACAATTCCATTCTATCTAA | 13 |
| 5_4 × 17 bp | CGGTTTGATAATCACTTGCCATTCTATCTAACGGTTTGATACAA | |
| | CTGGACCATTCTATCTAACGGTTTGA (SEQ ID NO: 64) | |
| synmiR- | CTTTTCTAGGTAATTTAGCAATACAACAATTCTTTTCTAGGTAAT | 14 |
| 7_4 × 12 bp | TTAGCAATAATCACTTGCTTTTCTAGGTAATTTAGCAATACAACT | |
| | GGACTTTTCTAGGTAATTTAGCAA (SEQ ID NO: 65) | |
| synmiR- | CTTTTCTAGGTAATTTAGCAATACAACAATTCTTTTCTAGGTAAT | 4F, 14 |
| 7_8 × 12 bp | TTAGCAATAATCACTTGCTTTTCTAGGTAATTTAGCAATACAACT | |
| | GGACTTTTCTAGGTAATTTAGCAATAACTAGTTCTTTGACTTTTCT | |
| | AGGTAATTTAGCAATACAACAATTCTTTTCTAGGTAATTTAGCA | |
| | ATAATCACTTGCTTTTCTAGGTAATTTAGCAATACAACTGGACTT | |
| | TTCTAGGTAATTTAGCAA (SEQ ID NO: 66) | |
| synmiR- | GTAACGTCAAATTAGATCGAATACAACAATTGTAACGTCAAATT | 14 |
| 8_4 × 8 bp | AGATCGAATAATCACTTGGTAACGTCAAATTAGATCGAATACAA | |
| | CTGGAGTAACGTCAAATTAGATCGAA (SEQ ID NO: 67) | |
| synmiR- | GTAACGTCAAATAAGATCGAATACAACAATTGTAACGTCAAATA | 14 |
| 8_4 × 9 bp | AGATCGAATAATCACTTGGTAACGTCAAATAAGATCGAATACAA | |
| | CTGGAGTAACGTCAAATAAGATCGAA (SEQ ID NO: 68) | |
| synmiR- | GTAACGTCAAATTAGATCGAATACAACAATTGTAACGTCAAATT | 4F, 14 |
| 8_8 × 8 bp | AGATCGAATAATCACTTGGTAACGTCAAATTAGATCGAATACAA | |
| | CTGGAGTAACGTCAAATTAGATCGAATAACTAGTTCTTTGAGTAA | |
| | CGTCAAATTAGATCGAATACAACAATTGTAACGTCAAATTAGAT | |
| | CGAATAATCACTTGGTAACGTCAAATTAGATCGAATACAACTGG | |
| | AGTAACGTCAAATTAGATCGAA (SEQ ID NO: 69) | |
| synmiR- | TCCAGCCTTTCTTAAAACTTATACAACAATTTCCAGCCTTTCTTA | 14 |
| 9_4 × 16 bp | AAACTTATAATCACTTGTCCAGCCTTTCTTAAAACTTATACAACT | |
| | GGATCCAGCCTTTCTTAAAACTTA (SEQ ID NO: 70) | |
| synmiR- | TCCAACCTTTCTTAAAACTTATACAACAATTTCCAACCTTTCTTA | 4F, 14 |
| 9_4 × 17 bp | AAACTTATAATCACTTGTCCAACCTTTCTTAAAACTTATACAACT | |
| | GGATCCAACCTTTCTTAAAACTTA (SEQ ID NO: 71) | |
| synmiR- | TCCCACCTTTCTTAAAACTTATACAACAATTTCCCACCTTTCTTA | 14 |
| 9_4 × 18 bp | AAACTTATAATCACTTGTCCCACCTTTCTTAAAACTTATACAACT | |
| | GGATCCCACCTTTCTTAAAACTTA (SEQ ID NO: 72) | |
| synmiR- | ATACCAAGACTCAATAGATGATACAACAATTATACCAAGACTCA | 4F, 14 |
| 10_8 × 8 bp | ATAGATGATAATCACTTGATACCAAGACTCAATAGATGATACAA | |
| | CTGGAATACCAAGACTCAATAGATGATAACTAGTTCTTTGAATAC | |
| | CAAGACTCAATAGATGATACAACAATTATACCAAGACTCAATAG | |
| | ATGATAATCACTTGATACCAAGACTCAATAGATGATACAACTGG | |
| | AATACCAAGACTCAATAGATGA (SEQ ID NO: 73) | |
| synmiR- | ATACCAAGACTCCATAGATGATACAACAATTATACCAAGACTCC | 14 |
| 10_8 × 9 bp | ATAGATGATAATCACTTGATACCAAGACTCCATAGATGATACAA | |
| | CTGGAATACCAAGACTCCATAGATGATAACTAGTTCTTTGAATAC | |
| | CAAGACTCCATAGATGATACAACAATTATACCAAGACTCCATAG | |
| | ATGATAATCACTTGATACCAAGACTCCATAGATGATACAACTGG | |
| | AATACCAAGACTCCATAGATGA (SEQ ID NO: 74) | |

Note:
the complementary sequences of the target are bolded.

TABLE 2
miRNAs

| miRNA/target name | Sequence |
|---|---|
| synmiR-1 | TCGTAGTTTATGCTTATGTTG (SEQ ID NO: 75) |
| synmiR-2_initial version | TTTATGTCACTTTGTAGATTA (SEQ ID NO: 76) |
| synmiR-2_20G | TTTATGTCACTTTGTAGATCA (SEQ ID NO: 77) |
| synmiR-3_initial version | TCAAGATATTATAAACTTTTC (SEQ ID NO: 78) |
| synmiR-3_19G | TCAAGATATTATAAACTTCTC (SEQ ID NO: 79) |
| synmiR-4 | TTCAATTTCATGATCTTTACG (SEQ ID NO: 80) |
| synmiR-5 | TCAAACCGTTAGATAGATGCT (SEQ ID NO: 81) |
| synmiR-6 | TATGAAACTGCTTGATAAGAG (SEQ ID NO: 82) |
| synmiR-7 | TTGCTAAATTACACCATTTCT (SEQ ID NO: 83) |
| synmiR-8 | TTCGATCTTTAAATCTCAACG (SEQ ID NO: 84) |
| synmiR-9 | TAAGTTTTAAGAAAGGTGTTG (SEQ ID NO: 85) |
| synmiR-10 | TCATCTATGCTTAAGATTACC (SEQ ID NO: 86) |
| miR-L | TAGATAAGCATTATAATTCCT (SEQ ID NO: 87) |

Some embodiments of the methods and compositions provided herein comprise circuits that have tunable expression of two or more payloads (e.g., a payload and one more supplemental payloads, secondary payloads). In some embodiments, the circuit comprises two or more distinct payloads regulated by distinct miRNAs (See Example 1). In some embodiments, the two or more payloads are regulated by the same miRNA. The two or more payloads can be regulated by the same miRNA in the circuit, but possess distinct miRNA target regions. The total of number of payload whose expression is regulated in the circuit can vary depending on the embodiment. In some embodiments, the composition, for each distinct supplemental miRNA, comprises: a supplemental first promoter sequence operably linked to a supplemental first polynucleotide comprising one or more supplemental miRNA cassettes, and a supplemental second promoter sequence operably linked to a supplemental second polynucleotide comprising a supplemental payload gene. In some embodiments, the supplemental first promoter sequence is capable of inducing transcription of the supplemental first polynucleotide to generate a supplemental first transcript, and wherein the supplemental first transcript is capable of being processed to generate said supplemental miRNA. In some embodiments, the supplemental payload gene comprises a supplemental miRNA target region comprising one or more supplemental miRNA target sequences, wherein the supplemental second promoter sequence is capable of inducing transcription of the supplemental second polynucleotide to generate a supplemental payload transcript, and wherein the supplemental payload transcript is capable of being translated to generate a supplemental payload protein. The secondary/supplemental payload proteins can comprise any of the payloads described herein. The payload protein and the one or more secondary proteins can be expressed as a fusion protein (and can be separated by one or more self-cleaving peptides). The 3′UTR of the transgene(s) encoding the one or more secondary proteins can comprise one or more miRNA binding sequences. The payload protein and the one or more secondary proteins can be expressed on separate payload transcripts. The payload protein and the one or more secondary proteins can be encoded on a single transcript, and wherein translations of the payload protein and the one or more secondary proteins can be each driven by a separate internal ribosome entry site. The sequences of the internal ribosome entry sites can be identical or different.

The nucleic acid composition can comprise, DNA, RNA, or a mixture of DNA and RNA. In some embodiments, the nucleic acid composition comprises one or more mRNAs (e.g., comprising a first promoter sequence operably linked to a first polynucleotide comprising one or more miRNA cassettes and a second promoter sequence operably linked to a second polynucleotide comprising a payload gene). In some embodiments, the composition is a vector, a ribonucleoprotein (RNP) complex, a liposome, a nanoparticle, an exosome, a microvesicle, or any combination thereof. In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the transposable element is piggybac transposon or sleeping beauty transposon. In some embodiments, the first polynucleotide and the second polynucleotide are comprised in the one or more vectors. In some embodiments, the first polynucleotide and the second polynucleotide are comprised in the same vector and/or different vectors. In some embodiments, the first polynucleotide and the second polynucleotide are situated on the same nucleic acid and/or different nucleic acids. In some embodiments, the nucleic acid composition comprises less than about 1.0 kb, 1.5 kb, 2.0 kb, 2.5 kb, 3.0 kb, 3.5 kb, 4.0 kb, 4.5 kb, 5.0 kb, 5.5 kb, 6.0 kb, 6.5 kb, 7.0 kb, 7.5 kb, 8.0 kb, 8.5 kb, 9.0 kb, 9.5 kb, 10 kb, 12 kb, 15 kb, 20 kb, 25 kb, 30 kb, 40 kb, 50 kb, 60 kb, 70 kb, 80 kb, 90 kb, or 100 kb. In some embodiments, the expression of the miRNA perturbs endogenous gene expression of the one or more cells less that about 10%. The method can comprise: one or more nucleic acids encoding TNRC6, GW182, one or more miRNA biogenesis components, Exportin-5, Dicer, and/or one or more Argonaute proteins. In some embodiments, the in the absence of a recombination event, the first promoter sequence and the first polynucleotide are not operably linked, and wherein the first promoter sequence and the first polynucleotide are operably linked after the recombination event such that the first promoter sequence is capable of inducing transcription of the first polynucleotide to generate a first transcript. In some embodiments, the first polynucleotide and the second polynucleotide are integrated in the genome of the one or more cells.

Disclosed herein include research methods. In some embodiments, the research method comprises: introducing into one or more cells a nucleic acid composition disclosed herein; and obtaining biological information of the one or more cells, optionally via sequencing or imaging. In some embodiments, the imaging comprises CRISPR imaging and/or super-resolution imaging, optionally DNA-PAINT (Point Accumulation for Imaging in Nanoscale Topography). Disclosed herein include production methods. In some embodiments, the production method comprises: introducing into one or more cells a nucleic acid composition disclosed herein; and isolating the payload protein or a payload associated therewith.

Disclosed herein include methods of treating a disease or disorder in a subject. In some embodiments, the method comprises: administering to the subject an effective amount of one or more cells disclosed herein, thereby treating or preventing the disease or disorder in the subject. Disclosed herein include methods of treating a disease or disorder in a subject. In some embodiments, the method comprises: introducing into one or more cells a nucleic acid composition disclosed herein; and administering to the subject an effective amount of the resulting cells, thereby treating or preventing the disease or disorder in the subject. The method can comprise: isolating the one or more cells from the subject prior to the introducing step. Disclosed herein include methods of treating a disease or disorder in a subject. In some embodiments, the method comprises: administering to the subject an effective amount of a nucleic acid composition disclosed herein, thereby treating or preventing the disease or disorder in the subject. Disclosed herein include methods for tuned dosage-invariant expression of a payload protein in one or more cells. In some embodiments, the method comprises: introducing into one or more cells a nucleic acid composition disclosed herein. In some embodiments, the introducing step is performed in vivo, in vitro, and/or ex vivo. In some embodiments, the introducing step comprises calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the one or more cells comprise one or more cells of a subject, optionally the subject is suffering from a disease or disorder. The method can comprise: introducing an inducer of the inducible promoter to the one or more cells, optionally the inducer comprises doxycycline, further optionally the introducing step comprises administering an initial dose of the inducer followed one or more lower maintenance doses of the inducer.

In some embodiments, the one or more cells comprise a eukaryotic cell, optionally the eukaryotic cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

The disease or disorder can be an expression-sensitive disease or disorder. An expression-sensitive disease or disorder can be characterized by decreased expression of one or more proteins, wherein ectopic overexpression of said one or more proteins at a steady state level beyond the upper tuned threshold causes cellular toxicity and/or disease. The disease or disorder can be a disease or disorder provided in Table 3. Table 3 provides an exemplary list of so-called “Goldilocks” diseases and disorders wherein disease phenotypes are attributable to decreased expression of genes but also exhibit cellular toxicity or outright disease when overexpressed. The methods and compositions provided herein are surprisingly capable of treating or preventing said diseases and disorders via the tunable and robust expression means provided herein. In some embodiments, the payload gene comprises any of the genes provided in Table 3.

TABLE 3
EXPRESSION-SENSITIVE DISEASES AND DISORDERS

| Disorder Class | Disorder | Gene Implicated in Disease |
|---|---|---|
| Neurodevelopmental | Rett Syndrome | MeCP2 |
| Syndromic Disorders | Smith-Magenis Syndrome | RAI1 |
| | Phelan-McDermid Syndrome | SHANK3 |
| | Cornelia de Lange Syndrome and other | NIPBL |
| | NIPBL related disorders | |
| | DRK1A, KAT6A and related disorders of | DRK1A, KAT6A |
| | severe intellectual disability | |
| | Chromosome 2Q37 Deletion Syndrome and | HDAC4 |
| | other HDAC4 Related Disorders | |
| | Angelman Syndrome | UBE3A |
| | Kleefstra Syndrome | EHMT1 and other genes |
| | | encoded on chromosome |
| | | 9q34.3 |
| | Joubert Syndrome and other NPHP1 Related | NPHP1 |
| | Disorders | |
| | Williams Syndrome | LIMK1 and other genes |
| | | encoded on chromosome |
| | | 7q11.23 |
| Proliferative/Cancer | Neurofibromatosis Type 1 | NF1 |
| Disorders | Li-Fraumeni syndrome and similar p53- | P53 |
| | related cancer syndromes | |
| Glycogen Storage | Phosphofructokinase Deficiency | PFK |
| Disorders | | |
| Hematologic/Immune | X-linked Hyper IgM Syndrome and similar | CD40L |
| Disorders | primary immunodeficiency disorders | |
| | Triosephosphate isomerase deficiency | TPI1 |
| Endocrine Disorders | Kallman Syndrome | FGFR1 and related genes |
| | Aromatase Deficiency | CYP19A1 |
| Other | Batten Disease, Frontotemporal Dementia | PGRN |
| Neuropsychiatric | and other neurodegenerative disorders | |
| Disorders | related to loss of progranulin | |
| | Cholinergic Receptor Nicotinic Alpha 7 | CHRNA7 |
| | Subunit Related Disorders | |
| | Hereditary Neuropathy with liability to | PMP22 |
| | Pressure Palsies | |

### Promoters

The promoters of the nucleic acids provided herein (e.g., first promoter sequence, second promoter sequence) can vary depending on the embodiment. The promoter can comprise a ubiquitous promoter. The ubiquitous promoter can be selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

In some embodiments, one or more cells of a subject (e.g., a human) comprise an endogenous version of the payload gene, and the promoter can comprise or can be derived from the promoter of the endogenous version. The promoter can comprise at least about 25% (e.g., 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, or a number or a range between any two of these values) homology to the promoter of the endogenous version of the payload gene. The promoter can be a methyl CpG binding protein 2 (MeCP2) promoter or a derivative thereof (e.g., a MeCP2 promoter truncated to about 229 bp and/or a MeCP2 promoter truncated to about 406 bp). The promoter can comprise an intronic sequence. The promoter can comprise a bidirectional promoter and/or an enhancer (e.g., a CMV enhancer).

The promoter can be an inducible promoter (e.g., a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, and/or an RU-486 responsive promoter). The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. The tissue specific promoter can be a neuron-specific promoter (e.g., a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ1.7 promoter, a Naᵥ1.8 promoter, a Naᵥ1.9 promoter, or an Advillin promoter). The tissue specific promoter can be a muscle-specific promoter (e.g., a creatine kinase (MCK) promoter).

In some embodiments, payload expression can be gated by a drug/small molecule. In some embodiments, the method can comprise an inducible promoter or a repressible promoter. The method can comprise administering one or more doses (e.g., a higher starting dose and a lower maintenance dose) of an agent that exerts an effect on the promoter (e.g., an inducer of said inducible promoter). In some such embodiments, only induce payload expression at a certain time point or expression profile, such as, for example, cases where a starting higher does versus a longer term maintenance dose is needed. Some embodiments of the compositions, methods, and systems provided herein can comprise one or more components of an transactivator rtTA (reverse tetracycline-controlled transactivator) system. By using an rrTA system, expression of the gene of interest (e.g. payload) can be further regulated by an inducible system whereby only when a small molecule doxycycline is added, the IFFL regulated construct is expressed.

The compositions provided herein can comprise a tetracycline-on (Tet-On) system. As an example, tetracycline-on (Tet-On) systems can use a reverse tetracycline transactivator (rtTA) to induce gene expression. Reverse tetracycline transactivators (rtTAs) comprise a mutant tetracycline repressor DNA binding protein (TetR) and a transactivation domain. These transactivators can be activated in the presence of a tetracycline (e.g., doxycycline) and subsequently bind to promoters comprising a tetracycline-responsive element (TRE) to induce gene expression. A TRE comprises at least one Tet operator (Tet-O) sequence (e.g., multiple repeats of Tel-0 sequences) and may be located upstream of a minimal promoter (e.g., minimal promoter sequence derived from the human cytomegalovirus (hCMV) immediate-early promoter). A “Tet-On” system, as used herein, is a type of inducible system that is capable of inducing expression of a particular payload gene in the presence of tetracycline (e.g., doxycycline (DOX)). In certain embodiments, a Tet-On system comprises a tetracycline-responsive promoter operably linked to a payload gene (e.g., a therapeutic sequence, a gene-targeting nucleic acid, and/or a nucleic acid encoding a protein) and a reverse tetracycline-controlled transactivator (rtTA). The expression cassette encoding a tetracycline-responsive promoter (e.g., a promoter comprising a TRE, including TRE3G, P tight, and TRE2) and a reverse tetracycline-controlled transactivator may be encoded on the same vector or be encoded on separate vectors. In some embodiments, the promoter comprises Tet Response Element (TRE). Tetracycline-dependent promoters can be constructed by placing a TRE upstream of a minimal promoter.

A “reverse tetracycline transactivator” (“rtTA”), as used herein, shall be given its ordinary meaning, and shall also refer an inducing agent that binds to a TRE promoter (e.g., a TRE3G, P tight, or TRE2 promoter) in the presence of tetracycline (e.g., doxycycline) and is capable of driving expression of a payload gene that is operably linked to the TRE promoter. rtTAs generally comprise a mutant tetracycline repressor DNA binding protein (TetR) and a transactivation domain. Any suitable transactivation domain may be used. Non-limiting examples include VP64, P65, RTA, and MPH MS2-P65-HSF1. In some embodiments, a rtTA of the present disclosure comprises at least 1, 2, 3, 4, 5, 6, 7, 8, 9, 10, 11, 12, 13, 14, 15, 16, 17, 18, 19, 20, 30, 40, 50, 60, 70, 80, 90, or 100 transactivation domains. The mutant TetR domain is capable of binding to a TRE promoter when bound to tetracycline.

The methods and compositions provided herein can comprise a tetracycline repressor. The term “tetracycline repressor” or “TetR” shall be given its ordinary meaning, and shall also refer to a protein that is capable of binding to a Tet-0 sequence (e.g., a Tet-0 sequence in a TRE) in the absence of tetracycline (e.g., doxycycline) and prevents binding of rtTA (e.g., rtTA3, rtTA4, or variants thereof) in the absence of tetracycline (e.g., doxycycline). TetRs prevent gene expression from promoters comprising a TRE in the absence of tetracycline (e.g., doxycycline). In the presence of tetracycline, TetRs cannot bind promoters comprising a TRE, and TetR cannot prevent transcription.

The term“promoter” shall be given its ordinary meaning, and shall also refer to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation of that sequence, expression of that sequence, or a combination thereof.

A promoter may promote ubiquitous expression or tissue-specific expression of an operably linked nucleic acid sequence from any species, including humans. In some embodiments, the promoter is a eukaryotic promoter. Non-limiting examples of eukaryotic promoters include TDH3, PGK1, PKC1, TDH2, PYK1, TPI1, AT1, CMV, EF1a, SV40, PGK1 (human or mouse), Ubc, human beta actin, CAG, TRE, UAS, Ac5, Polyhedrin, CaMKIIa, GAL1, GAL 10, TEF1, GDS, ADH1, CaMV35S, Ubi, Hl, and U6, as would be known to one of ordinary skill in the art.

Non-limiting examples of ubiquitous promoters include tetracycline-responsive promoters (under the relevant conditions), CMV, EF1 alpha, a SV40 promoter, PGK1, Ubc, CAG, human beta actin gene promoter, and a promoter comprising an upstream activating sequence (UAS). In certain embodiments, the promoter is a mammalian promoter. Non-limiting examples of tissue-specific promoters include brain-specific, liver-specific, muscle-specific, nerve cell-specific, lung-specific, heart-specific, bone-specific, intestine-specific, skin-specific promoters, brain-specific promoters, and eye-specific promoters.

Non-limiting examples of constitutive promoters include CPT, CMV, EF1 alpha, SV40, PGK1, Ubc, human beta actin, beta tubulin, CAG, Ac5, polyhedrin, TEF1, GDS, CaM3 5S, Ubi, Hl, and U6.

An “inducible promoter” shall be given its ordinary meaning, and shall also refer one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid in such a way as to be active in inducing transcriptional activity from the inducible promoter. In certain embodiments, an inducing agent is a tetracycline-sensitive protein (e.g., rtTA).

Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetRTetR), a tetracycline operator sequence (tetO), and a tetracycline transactivator fusion protein (tTA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters.

### Payloads

In some embodiments, the payload protein comprises a disease-associated protein, wherein aberrant expression of the disease-associated protein correlates with the occurrence and/or progression of the disease. The payload protein can comprise a protein associated with an expression-sensitive disease or disorder as provided in Table 3. The payload protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof.

The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof.

The payload can comprise one or more components (e.g., a programmable nuclease, PegRNA, sgRNA) of a gene editing system (e.g., a CRISPR-CAS9 editing (Clustered Regularly Interspaced Short Palindromic Repeats (CRISPR)-CRISPR-associated protein 9 (Cas9)) system, single base editing, or prime editing) configured to remove or edit one or more nucleobases, e.g., to substitute, insert, and/or delete sequences present in the genome of a subject or patient. CRISPR-Cas9 may be used to inactivate or correct a gene, or base editors or prime editors may be utilized. The CRISPR-Cas9 system, as well as single base editors, include a guide RNA (gRNA) or single guide RNA (sgRNA) and a CRISPR-associated protein 9 (Cas9) nuclease. Identification of the DNA target strand, and methods of implementing a change in the target DNA (e.g., gene knock out in the target DNA strand, knock-in of a desired sequence, or base substitutions) are within the abilities of one of ordinary skill in the art. The circuits provided herein can provide tunable dosage invariant expression of gene editing system component(s), and can, in some embodiments, yield reduced off target editing effects as compared to currently available approaches of expressing gene editing system component(s).

There are provided, in some embodiments, methods and compositions for self-regulated gene therapy that can provide regulated expression independent of gene dosage. In some embodiments, a self-regulated gene therapy payload can provide regulated expression of a payload (e.g., MeCP2) independent of gene dosage. In some embodiments, the methods and compositions provide dosage invariance of a payload. Dosage invariance, as used herein, shall be given its ordinary meaning, and shall also refer to a lower fold change in protein output compared to gene dosage. The methods and compositions provided herein can yield and at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) lower fold change in protein output as compared to gene dosage. In some embodiments, the methods and compositions provided here enable payload dosage invariance in vivo (e.g., in a mouse, in a human).

In Crispr-Cas9 editing, when the Cas9 nuclease binds with the PAM and the gRNA binds with the target DNA strand, a double-strand break is caused in the gRNA sequence. Endogenous repair mechanisms, such as non-homologous end joining (NHEJ), microhomology-mediated end joining MMEJ), or homologous directed repair (HDR), are triggered by the double-strand break and result in a gene knock out in the target DNA strand or a knock-in of a desired sequence if a DNA template is present. The DNA template includes the desired sequence, which is flanked by sequences that are homologous to the region upstream and downstream of the double-stranded break. The gRNA includes a CRISPR RNA (crRNA), which is a 17-20 nucleotide sequence that is complementary to the target DNA strand, and a tracrRNA, which serves as a binding scaffold for the Cas9 nuclease. The crRNA and the tracrRNA may exist as two separate RNA molecules. Alternatively, the sgRNA may comprise both the crRNA sequence and the tracrRNA sequence, where the crRNA sequence is fused to the scaffold tracrRNA sequence. gRNAs of base editing methods as described below, have canonical structures specific to each technique. One of ordinary skill in the art would select a gRNA or sgRNA that maximizes the on-target DNA cleavage efficiency, while also minimizing unintentional off-target binding and cleavage effects.

Base editing is a genome-editing technique that uses DNA base editors to directly generate precise point mutations without generating a double-strand break without double-strand breaks. The DNA base editors may comprise fusions between a catalytically dead Cas9 (dCas9) or a nickase Cas9 (nCas9) fused to a single-stranded DNA (ssDNA)-specific deaminase and a single guide RNA (sgRNA). The d/nCas9 recognizes a specific sequence named protospacer adjacent motif (PAM) and the DNA unwinds thanks to the complementarity between the sgRNA and the DNA sequence usually located upstream of the PAM (“protospacer”). Then, the opposite DNA strand is accessible to the deaminase that converts the bases located in a specific DNA stretch of the protospacer (see, e.g., Antoniou P, et al. Base and Prime Editing Technologies for Blood Disorders. Front Genome Ed. 2021 Jan. 28; 3:618406). Upon binding of the DNA base editor to the target DNA strand, base pairing between the sgRNA and the target DNA strand results in the displacement of a small segment of ssDNA as an “R-loop”. The DNA bases within the ssDNA are therefore substrates for deamination and are subsequently modified by the deaminase enzyme. The DNA base editor may be a cytosine base editor (CBE), which converts a C/G base pair into a T/A base pair or an adenine base editor (ABE) which converts an A/T base pair into a G/C base pair.

Another method of gene editing is prime editing, which is disclosed in U.S. Pat. No. 11,447,770 B1, incorporated herein by reference for its technical disclosure, and related publications (see also, International Patent Publication No. WO2020191242 A1 and Anzalone A V, et al. Search-and-replace genome editing without double-strand breaks or donor DNA. Nature. 2019 December; 576(7785):149-157). Prime editors (PEs), including a complete description of pegRNA are provided in those references, as well as methods of in vivo delivery of prime editor materials, such as viral vectors, e.g., AAV particles encoding prime editors, are described in that patent publication and related publications. Prime editing is a “search and replace” gene editing method in which Moloney Murine Leukemia Virus Reverse Transcriptase (M-MLV RT) is fused to the C-terminus of Cas9 H840A nickase. The fusion enzyme is installs targeted insertions, deletions, and all possible base-to-base conversions using a prime editing guide RNA (pegRNA). The pegRNA directs the nickase to the target site by homology to a genomic DNA locus. The longer pegRNA also encodes a primer binding site (PBS) and the desired edits on an RT template. Prime editing has gone through a number of versions. In PE1, the pegRNA directs the Cas9 nickase to the target sequence where it nicks the non-target strand and generates a 3′ flap. The 3′ flap binds to the primer binding site (PBS) of the pegRNA and the desired edit is incorporated into the DNA by reverse transcription. The edited DNA strand displaces the unedited 5′ flap and the resulting heteroduplex is resolved by the cell's mismatch repair (MMR) system. Alternatively, the edited 3′ flap may be excised and the target sequence will remain unchanged but available as a substrate for another round of prime editing. In the PE2 system, mutations were introduced into the RT enzyme to increase activity, enhance binding between the template and PBS, increase processivity, and improve thermostability. PE3 uses the PE2 Cas9 nickase-pentamutant RT fusion enzyme and pegRNA plus an additional simple sgRNA, which directs the Cas9 nickase to nick the unedited strand at a nearby site. The newly edited strand is then favored as the template for repair during heteroduplex resolution. The process of double nicking, however, increases indel formation slightly. Designing the sgRNA with a spacer that only binds the edited strand, as in the PE3b system, guides nicking of the unedited strand only after the edit has occurred. PE4 and PE5 also have been described (Chen P J, et al. Enhanced prime editing systems by manipulating cellular determinants of editing outcomes. Cell. 2021 Oct. 28; 184(22):5635-5652.e29). “Prime editing” includes all variations of prime editing methods, including, without limitation, PE1, PE2, PE3, PE3b, PE4, and PE5 versions. pegRNA includes variations thereof for use in the many variations of prime editing, such as, without limitation, epegRNA (Nelson J W, et al. Engineered pegRNAs improve prime editing efficiency. Nat Biotechnol. 2022 March; 40(3):402-410).

The payload protein can comprise a programmable nuclease. The programmable nuclease can be selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the nucleic acid further comprises a polynucleotide encoding (i) a targeting molecule and/or (ii) a donor nucleic acid. The composition can comprise (i) a targeting molecule or a nucleic acid encoding the targeting molecule and/or (ii) a donor nucleic acid or a nucleic acid encoding the donor nucleic acid. The targeting molecule can be capable of associating with the programmable nuclease. The targeting molecule can comprise single strand DNA or single strand RNA. The targeting molecule can comprise a single guide RNA (sgRNA). The programmable nuclease can comprise a zinc finger nuclease, TAL effector nuclease, meganclease, MegaTAL, Tev-m TALEN, MegaTev, homing endonuclease, derivatives thereof, or any combination thereof. The targeting molecule can be capable of associating with the programmable nuclease. The targeting molecule can comprise single strand DNA or single strand RNA. The targeting molecule can comprise a single guide RNA (sgRNA). The targeting molecule can comprise a synthetic nucleic acid.

In some embodiments, the payload comprises one or more programmable nucleases disclosed in Table 4. In some embodiments, the payload comprises one or more prime editors. There are provided, in some embodiments, methods of gene editing that fulfill the gene purpose and/or regulation purpose set forth in Table 4.

TABLE 4
PROGRAMMABLE NUCLEASES

| Gene | Gene Purpose | Regulation Purpose |
|---|---|---|
| SpCas9 | Genome Engineering, | Reduce off-target nuclease activity or |
| | gene editing | immunogenicity |
| mutations thereof: | | |
| VRER, VQR, and EQR | Genome Engineering, | Reduce off-target nuclease activity or |
| SpCas9 | gene editing | immunogenicity |
| xCas9-3.7 | | |
| eSpCas9 | | |
| Cas9-HF1 | | |
| HypaCas9 | | |
| evoCas9 | | |
| HiFi Cas9 | | |
| Other Cas9 species: | | |
| ScCas9 | Genome Engineering, | Reduce off-target nuclease activity or |
| StCas9 | gene editing | immunogenicity |
| NmCas9 | | |
| SaCas9 | | |
| CjCas9 | | |
| CasX | | |
| Cas9 H940A nickase | Prime editing | Reduce off-target editing and |
| | | immunogenicity. |
| Cas12 and mutations | Multiplex gene editing | Reduce off-target nuclease activity or |
| | | immunogenicity |
| dcas9-APOBEC1 fusion, BE3, | CRISPR base editing | Reduce off-target base editing (which |
| other dcas9-deaminase fusions | | is a significant unsolved problem) or |
| | | immunogenicity |
| dcas9-Krab, dCas9-VP64, | activate/repress | Reduce off-target, decrease |
| dCas9-Tet1, and other dcas9- | transcription, modify | immunogenicity. |
| transcriptional regulator fusion | epigenetic state | |
| Dcas9-fluorescent protein | Imaging and tracking | Increase signal to noise ratio |
| fusions | genomic loci and | |
| | chromatin dynamics | |
| Cas13-fluorescent protein | RNA imaging and | Increase signal to noise ratio |
| fusions | tracking | |
| RCas9-fluorescent protein | RNA imaging | Increase signal to noise ratio |
| fusions | | |
| Cas13-adenosine deaminase | RNA editing | reduce off-target and immunogenicity |
| fusions | | |

The payload protein can comprise a chimeric antigen receptor. The methods and compositions provided herein find use in cell therapies (e.g., adoptive therapies). The methods and compositions provided herein can involve the adoptive transfer of immune system cells, such as T cells, specific for selected antigens, such as tumor associated antigens. Various strategies may, for example, be employed to genetically modify T cells by altering the specificity of the T cell receptor (TCR) for example by introducing payloads comprising new TCR a and b chains with selected peptide specificity. As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described. The methods and compositions provided herein can involve adoptive immunotherapy, comprising (1) knock-in an exogenous gene encoding a chimeric antigen receptor (CAR) or a T-cell receptor (TCR), (2) knock-out or knock-down expression of an immune checkpoint receptor, (3) knock-out or knock-down expression of an endogenous TCR, (4) knock-out or knock-down expression of a human leukocyte antigen class I (HLA-I) proteins, and/or (5) knock-out or knock-down expression of an endogenous gene encoding an antigen targeted by an exogenous CAR or TCR.

The payload protein can be associated with an agricultural trait of interest selected from the group consisting of increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof.

The payload protein can be associated with a biological manufacturing process selected from the group comprising fermentation, distillation, biofuel production, production of a compound, production of a polypeptide, or any combination thereof.

The payload can comprise non-coding (e.g. lncRNA) genes. The payload can be an RNA gene product. The one or more payload genes of the nucleic acid can comprise a siRNA, a shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof.

The payload protein can be any protein, including naturally-occurring and non-naturally occurring proteins. Examples of payload protein include, but are not limited to, luciferases; fluorescent proteins (e.g., GFP); growth hormones (GHs) and variants thereof; insulin-like growth factors (IGFs) and variants thereof; granulocyte colony-stimulating factors (G-CSFs) and variants thereof; erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; cystic fibrosis transmembrane conductance regulator (CFTR) and variants thereof; and interferons and variants thereof.

In some embodiments, the payload protein is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein is an active fragment of a protein, such as any of the aforementioned proteins. In some embodiments, the payload protein is a fusion protein comprising some or all of two or more proteins. In some embodiments a fusion protein can comprise all or a portion of any of the aforementioned proteins.

In some embodiments, the payload protein is a multi-subunit protein. For examples, the payload protein can comprise two or more subunits, or two or more independent polypeptide chains. In some embodiments, the payload protein can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, the payload gene encodes a pro-survival protein (e.g., Bcl-2, Bcl-XL, Mcl-1 and A1). In some embodiments, the payload gene encodes a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-xL, Bcl-xs, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, the payload gene encodes a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. In some embodiments, the payload gene encodes a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, the payload gene encodes a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1INH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typlll repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

In some embodiments, the payload gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the nucleic acids provided herein can be employed as vaccines for the prophylaxis or treatment of infectious diseases (e.g., as vaccines).

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload gene expression in the target cell can vary. For example, in some embodiments, the payload gene encodes a payload protein. The amount of the payload protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 g/ml, at least about 700 g/ml, at least about 800 g/ml, at least about 900 g/ml, or at least about 1000 g/ml. In some embodiments, the payload protein is expressed in the serum of the subject in the amount of about 9 μg/ml, about 10 μg/ml, about 50 μg/ml, about 100 μg/ml, about 200 g/ml, about 300 g/ml, about 400 g/ml, about 500 g/ml, about 600 g/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation.

A payload protein encoded by a payload gene can be of various lengths. For example, the payload protein can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. In some embodiments, the payload protein is at least about 480 amino acids in length. In some embodiments, the payload protein is at least about 500 amino acids in length. In some embodiments, the payload protein is about 750 amino acids in length.

The payload genes can have different lengths in different implementations. The number of payload genes can be different in different embodiments. In some embodiments, the number of payload genes in a nucleic acid can be, or can be about, 1, 2, 3, 4, 5, 6, 7, 8, 9, 10, 11, 12, 13, 14, 15, 16, 17, 18, 19, 20, 21, 22, 23, 24, 25, or a number or a range between any two of these values. In some embodiments, the number of payload genes in a nucleic acid can be at least, or can be at most, 1, 2, 3, 4, 5, 6, 7, 8, 9, 10, 11, 12, 13, 14, 15, 16, 17, 18, 19, 20, 21, 22, 23, 24, 25. In some embodiments, a payload genes is, or is about, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, nucleotides in length. In some embodiments, a payload gene is at least, or is at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000 nucleotides in length.

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload can activate an adaptive immune response, and innate immune response, or both. In some embodiments, the payload gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the compositions provided herein can be employed as vaccines for the prophylaxis or treatment of infectious diseases (e.g., as vaccines). The payload protein can comprise a CRE recombinase, GCaMP, a cell therapy component, a knock-down gene therapy component, a cell-surface exposed epitope, or any combination thereof.

Examples of payload genes include a sequence associated with a signaling biochemical pathway, e.g., a signaling biochemical pathway-associated gene or polynucleotide (e.g., a signal transducer). In some embodiments, the methods and compositions disclosed herein comprise knockdown of an endogenous signal transducer accompanied by tuned expression of a payload protein comprising an appropriate version of signal transducer. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level. Signal transducers can be can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 5-7. In some embodiments, the methods and compositions disclosed herein prevent or treat one or more of the diseases and disorders listed in Tables 5-7. In some embodiments, the payload comprises a replacement version of the signal transducer. In some embodiments, the methods and compositions further comprise knockdown of the corresponding endogenous signal transducer. The payload can comprise the product of a gene listed in listed in Tables 5-7. In some embodiments, the payload ameliorates a disease or disorder characterized by an aberrant signaling of one or more signaling transducers. In some embodiments, the payload diminishes the activation level of one or more signal transducers (e.g., signal transducers with aberrant overactive signaling, signal transducers listed in Tables 5-7). In some embodiments, the payload increases the activation level of one or more signal transducers (e.g., signal transducers with aberrant underactive signaling). In some such embodiments, the payload can modulate the abundance, location, stability, and/or activity of activators or repressors of said signal transducers.

TABLE 5
DISEASES AND DISORDERS OF INTEREST

| Diseases/Disorders | Genes |
|---|---|
| Neoplasia | PTEN; ATM; ATR; EGFR; ERBB2; ERBB3; ERBB4; Notch1; Notch2; Notch3; |
| | Notch4; AKT; AKT2; AKT3; HIF; HIF1a; HIF3a; Met; HRG; Bcl2; PPAR alpha; |
| | PPAR gamma; WT1 (Wilms Tumor); FGF Receptor Family members (5 |
| | members: 1, 2, 3, 4, 5); CDKN2a; APC; RB (retinoblastoma); MEN1; VHL; |
| | BRCA1; BRCA2; AR (Androgen Receptor); TSG101; IGF; IGF Receptor; Igf1 (4 |
| | variants); Igf2 (3 variants); Igf 1 Receptor; Igf 2 Receptor; Bax; Bcl2; caspases |
| | family (9 members: 1, 2, 3, 4, 6, 7, 8, 9, 12); Kras; Apc |
| Age-related Macular | Abcr; Ccl2; Cc2; cp (ceruloplasmin); Timp3; cathepsinD; Vldlr; Ccr2 |
| Degeneration | |
| Schizophrenia | Neuregulin1 (Nrg1); Erb4 (receptor for Neuregulin); Complexin1 (Cplx1); Tph1 |
| | Tryptophan hydroxylase; Tph2 Tryptophan hydroxylase 2; Neurexin 1; GSK3; |
| | GSK3a; GSK3b |
| Disorders | 5-HTT (Slc6a4); COMT; DRD (Drd1a); SLC6A3; DAOA; DTNBP1; Dao (Dao1) |
| Trinucleotide Repeat | HTT (Huntington's Dx); SBMA/SMAX1/AR (Kennedy's Dx); FXN/X25 |
| Disorders | (Friedrich's Ataxia); ATX3 (Machado- Joseph's Dx); ATXN1 and ATXN2 |
| | (spinocerebellar ataxias); DMPK (myotonic dystrophy); Atrophin-1 and Atn1 |
| | (DRPLA Dx); CBP (Creb-BP - global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); PEN-2 |
| Disorders | |
| Others | Nos1; Parp1; Nat1; Nat2 |
| Prion-related disorders | Prp |
| ALS | SOD1; ALS2; STEX; FUS; TARDBP; VEGF (VEGF-a; VEGF-b; VEGF-c) |
| Drug addiction | Prkce (alcohol); Drd2; Drd4; ABAT (alcohol); GRIA2; Grm5; Grin1; Htr1b; |
| | Grin2a; Drd3; Pdyn; Gria1 (alcohol) |
| Autism | Mecp2; BZRAP1; MDGA2; Sema5A; Neurexin 1; Fragile X (FMR2 (AFF2); |
| | FXR1; FXR2; Mglur5) |
| Alzheimer's Disease | E1; CHIP; UCH; UBB; Tau; LRP; PICALM; Clusterin; PS1; SORL1; CR1; |
| | Vldlr; Uba1; Uba3; CHIP28 (Aqp1, Aquaporin 1); Uchl1; Uchl3; APP |
| Inflammation | IL-10; IL-1 (IL-1a; IL-1b); IL-13; IL-17 (IL-17a (CTLA8); IL- 17b; IL-17c; IL- |
| | 17d; IL-17f); II-23; Cx3cr1; ptpn22; TNFa; NOD2/CARD15 for IBD; IL-6; IL-12 |
| | (IL-12a; IL-12b); CTLA4; Cx3cl1 |
| Parkinson's Disease | x-Synuclein; DJ-1; LRRK2; Parkin; PINK1 |

TABLE 6
SIGNAL TRANSDUCERS

| | |
|---|---|
| Blood and | Anemia (CDAN1, CDA1, RPS19, DBA, PKLR, PK1, NT5C3, UMPH1, PSN1, RHAG, |
| coagulation | RH50A, NRAMP2, SPTB, ALAS2, ANH1, ASB, ABCB7, ABC7, ASAT); Bare |
| diseases and | lymphocyte syndrome (TAPBP, TPSN, TAP2, ABCB3, PSF2, RING11, MHC2TA, |
| disorders | C2TA, RFX5, RFXAP, RFX5); Bleeding disorders (TBXA2R, P2RX1, P2X1); Factor |
| | H and factor H-like 1 (HF1, CFH, HUS); Factor V and factor VIII (MCFD2); Factor |
| | VII deficiency (F7); Factor X deficiency (F10); Factor XI deficiency (F11); Factor XII |
| | deficiency (F12, HAF); Factor XIIIA deficiency (F13A1, F13A); Factor XIIIB |
| | deficiency (F13B); Fanconi anemia (FANCA, FACA, FA1, FA, FAA, FAAP95, |
| | FAAP90, FLJ34064, FANCB, FANCC, FACC, BRCA2, FANCD1, FANCD2, FANCD, |
| | FACD, FAD, FANCE, FACE, FANCF, XRCC9, FANCG, BRIP1, BACH1, FANCJ, |
| | PHF9, FANCL, FANCM, KIAA1596); Hemophagocytic lymphohistiocytosis disorders |
| | (PRF1, HPLH2, UNC13D, MUNC13-4, HPLH3, HLH3, FHL3); Hemophilia A (F8, |
| | F8C, HEMA); Hemophilia B (F9, HEMB), Hemorrhagic disorders (PI, ATT, F5); |
| | Leukocyde deficiencies and disorders (ITGB2, CD18, LCAMB, LAD, EIF2B1, |
| | EIF2BA, EIF2B2, EIF2B3, EIF2B5, LVWM, CACH, CLE, EIF2B4); Sickle cell anemia |
| | (HBB); Thalassemia (HBA2, HBB, HBD, LCRB, HBA1). |
| Cell | B-cell non-Hodgkin lymphoma (BCL7A, BCL7); Leukemia (TAL1 TCL5, SCL, TAL2, |
| dysregulation | FLT3, NBS1, NBS, ZNFN1A1, IK1, LYF1, HOXD4, HOX4B, BCR, CML, PHL, ALL, |
| and oncology | ARNT, KRAS2, RASK2, GMPS, AF10, ARHGEF12, LARG, KIAA0382, CALM, |
| diseases and | CLTH, CEBPA, CEBP, CHIC2, BTL, FLT3, KIT, PBT, LPP, NPM1, NUP214, D9S46E, |
| disorders | CAN, CAIN, RUNX1, CBFA2, AML1, WHSC1L1, NSD3, FLT3, AF1Q, NPM1, |
| | NUMA1, ZNF145, PLZF, PML, MYL, STAT5B, AF10, CALM, CLTH, ARL11, |
| | ARLTS1, P2RX7, P2X7, BCR, CML, PHL, ALL, GRAF, NF1, VRNF, WSS, NFNS, |
| | PTPN11, PTP2C, SHP2, NS1, BCL2, CCND1, PRAD1, BCL1, TCRA, GATA1, GF1, |
| | ERYF1, NFE1, ABL1, NQO1, DIA4, NMOR1, NUP214, D9S46E, CAN, CAIN). |
| Inflammation | AIDS (KIR3DL1, NKAT3, NKB1, AMB11, KIR3DS1, IFNG, CXCL12, SDF1); |
| and immune | Autoimmune lymphoproliferative syndrome (TNFRSF6, APT1, FAS, CD95, |
| related | ALPS1A); Combined immunodeficiency, (IL2RG, SCIDX1, SCIDX, IMD4); HIV-1 |
| diseases and | (CCL5, SCYA5, D17S136E, TCP228), HIV susceptibility or infection (IL10, CSIF, |
| disorders | CMKBR2, CCR2, CMKBR5, CCCKR5 (CCR5)); Immunodeficiencies (CD3E, CD3G, |
| | AICDA, AID, HIGM2, TNFRSF5, CD40, UNG, DGU, HIGM4, TNFSF5, CD40LG, |
| | HIGM1, IGM, FOXP3, IPEX, AIID, XPID, PIDX, TNFRSF14B, TACI); Inflammation |
| | (IL-10, IL-1 (IL-1a, IL-1b), IL-13, IL-17 (IL-17a (CTLA8), IL-17b, IL-17c, IL-17d, IL- |
| | 17f), 11-23, Cx3cr1, ptpn22, TNFa, NOD2/CARD15 for IBD, IL-6, IL-12 (IL-12a, IL- |
| | 12b), CTLA4, Cx3cl1); Severe combined immunodeficiencies (SCIDs)(JAK3, JAKL, |
| | DCLRE1C, ARTEMIS, SCIDA, RAG1, RAG2, ADA, PTPRC, CD45, LCA, IL7R, |
| | CD3D, T3D, IL2RG, SCIDX1, SCIDX, IMD4). |
| Metabolic, | Amyloid neuropathy (TTR, PALB); Amyloidosis (APOA1, APP, AAA, CVAP, AD1, |
| liver, kidney | GSN, FGA, LYZ, TTR, PALB); Cirrhosis (KRT18, KRT8, CIRH1A, NAIC, TEX292, |
| and protein | KIAA1988); Cystic fibrosis (CFTR, ABCC7, CF, MRP7); Glycogen storage diseases |
| diseases and | (SLC2A2, GLUT2, G6PC, G6PT, G6PT1, GAA, LAMP2, LAMPB, AGL, GDE, GBE1, |
| disorders | GYS2, PYGL, PFKM); Hepatic adenoma (TCF1, HNF1A, MODY3), Hepatic failure, |
| | early onset, and neurologic disorder (SCOD1, SCO1), Hepatic lipase deficiency |
| | (LIPC), Hepatoblastoma, cancer and carcinomas (CTNNB1, PDGFRL, PDGRL, |
| | PRLTS, AXIN1, AXIN, CTNNB1, TP53, P53, LFS1, IGF2R, MPRI, MET, CASP8, |
| | MCH5); Medullary cystic kidney disease (UMOD, HNFJ, FJHN, MCKD2, |
| | ADMCKD2); Phenylketonuria (PAH, PKU1, QDPR, DHPR, PTS); Polycystic kidney |
| | and hepatic disease (FCYT, PKHD1, ARPKD, PKD1, PKD2, PKD4, PKDTS, PRKCSH, |
| | G19P1, PCLD, SEC63). |
| Muscular/ | Becker muscular dystrophy (DMD, BMD, MYF6), Duchenne Muscular Dystrophy |
| Skeletal | (DMD, BMD); Emery-Dreifuss muscular dystrophy (LMNA, LMN1, EMD2, FPLD, |
| diseases and | CMD1A, HGPS, LGMD1B, LMNA, LMN1, EMD2, FPLD, CMD1A); |
| disorders | Facioscapulohumeral muscular dystrophy (FSHMD1A, FSHD1A); Muscular |
| | dystrophy (FKRP, MDC1C, LGMD2I, LAMA2, LAMM, LARGE, KIAA0609, MDC1D, |
| | FCMD, TTID, MYOT, CAPN3, CANP3, DYSF, LGMD2B, SGCG, LGMD2C, DMDA1, |
| | SCG3, SGCA, ADL, DAG2, LGMD2D, DMDA2, SGCB, LGMD2E, SGCD, SGD, |
| | LGMD2F, CMD1L, TCAP, LGMD2G, CMD1N, TRIM32, HT2A, LGMD2H, FKRP, |
| | MDC1C, LGMD2I, TTN, CMD1G, TMD, LGMD2J, POMT1, CAV3, LGMD1C, |
| | SEPN1, SELN, RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, TIRC7, OC116, |
| | OPTB1); Muscular atrophy (VAPB, VAPC, ALS8, SMN1, SMA1, SMA2, SMA3, |
| | SMA4, BSCL2, SPG17, GARS, SMAD1, CMT2D, HEXB, IGHMBP2, SMUBP2, |
| | CATF1, SMARD1). |
| Neurological | ALS (SOD1, ALS2, STEX, FUS, TARDBP, VEGF (VEGF-a, VEGF-b, VEGF-c); |
| and neuronal | Alzheimer disease (APP, AAA, CVAP, AD1, APOE, AD2, PSEN2, AD4, STM2, |
| diseases and | APBB2, FE65L1, NOS3, PLAU, URK, ACE, DCP1, ACE1, MPO, PACIP1, PAXIP1L, |
| disorders | PTIP, A2M, BLMH, BMH, PSEN1, AD3); Autism (Mecp2, BZRAP1, MDGA2, |
| | Sema5A, Neurexin 1, GLO1, MECP2, RTT, PPMX, MRX16, MRX79, NLGN3, NLGN4, |
| | KIAA1260, AUTSX2); Fragile X Syndrome (FMR2, FXR1, FXR2, mGLUR5); |
| | Huntington's disease and disease like disorders (HD, IT15, PRNP, PRIP, JPH3, JP3, |
| | HDL2, TBP, SCA17); Parkinson disease (NR4A2, NURR1, NOT, TINUR, SNCAIP, |
| | TBP, SCA17, SNCA, NACP, PARK1, PARK4, DJ1, PARK7, LRRK2, PARK8, PINK1, |
| | PARK6, UCHL1, PARK5, SNCA, NACP, PARK1, PARK4, PRKN, PARK2, PDJ, DBH, |
| | NDUFV2); Rett syndrome (MECP2, RTT, PPMX, MRX16, MRX79, CDKL5, STK9, |
| | MECP2, RTT, PPMX, MRX16, MRX79, x-Synuclein, DJ-1); Schizophrenia |
| | (Neuregulin1 (Nrg1), Erb4 (receptor for Neuregulin), Complexin1 (Cplx1), Tph1 |
| | Tryptophan hydroxylase, Tph2, Tryptophan hydroxylase 2, Neurexin 1, GSK3, GSK3a, |
| | GSK3b, 5-HTT (Slc6a4), COMT, DRD (Drd1a), SLC6A3, DAOA, DTNBP1, Dao |
| | (Dao1)); Secretase Related Disorders (APH-1 (alpha and beta), Presenilin (Psen1), |
| | nicastrin, (Ncstn), PEN-2, Nos1, Parp1, Nat1, Nat2); Trinucleotide Repeat Disorders |
| | (HTT (Huntington's Dx), SBMA/SMAX1/AR (Kennedy's Dx), FXN/X25 (Friedrich's |
| | Ataxia), ATX3 (Machado- Joseph's Dx), ATXN1 and ATXN2 (spinocerebellar ataxias), |
| | DMPK (myotonic dystrophy), Atrophin-1 and Atn1 (DRPLA Dx), CBP (Creb-BP - global |
| | instability), VLDLR (Alzheimer's), Atxn7, Atxn10). |
| Ocular | Age-related macular degeneration (Abcr, Ccl2, Cc2, cp (ceruloplasmin), Timp3, |
| diseases and | cathepsinD, Vldlr, Ccr2); Cataract (CRYAA, CRYA1, CRYBB2, CRYB2, PITX3, |
| disorders | BFSP2, CP49, CP47, CRYAA, CRYA1, PAX6, AN2, MGDA, CRYBA1, CRYB1, |
| | CRYGC, CRYG3, CCL, LIM2, MP19, CRYGD, CRYG4, BFSP2, CP49, CP47, HSF4, |
| | CTM, HSF4, CTM, MIP, AQP0, CRYAB, CRYA2, CTPP2, CRYBB1, CRYGD, |
| | CRYG4, CRYBB2, CRYB2, CRYGC, CRYG3, CCL, CRYAA, CRYA1, GJA8, CX50, |
| | CAE1, GJA3, CX46, CZP3, CAE3, CCM1, CAM, KRIT1); Corneal clouding and |
| | dystrophy (APOA1, TGFBI, CSD2, CDGG1, CSD, BIGH3, CDG2, TACSTD2, TROP2, |
| | M1S1, VSX1, RINX, PPCD, PPD, KTCN, COL8A2, FECD, PPCD2, PIP5K3, CFD); |
| | Cornea plana congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, NTG, NPG, |
| | CYP1B1, GLC3A); Leber congenital amaurosis (CRB1, RP12, CRX, CORD2, CRD, |
| | RPGRIP1, LCA6, CORD9, RPE65, RP20, AIPL1, LCA4, GUCY2D, GUC2D, LCA1, |
| | CORD6, RDH12, LCA3); Macular dystrophy (ELOVL4, ADMD, STGD2, STGD3, |
| | RDS, RP7, PRPH2, PRPH, AVMD, AOFMD, VMD2). |

TABLE 7
SIGNAL TRANSDUCTION PATHWAYS

| Pathway | Genes |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; ITGA1; KRAS; EIF4EBP1; RELA; PRKCD; NOS3; PRKAA1; MAPK9; |
| | CDK2; PPP2CA; PIM1; ITGB7; YWHAZ; ILK; TP53; RAF1; IKBKG; RELB; |
| | DYRK1A; CDKN1A; ITGB1; MAP2K2; JAK1; AKT1; JAK2; PIK3R1; |
| | CHUK; PDPK1; PPP2R5C; CTNNB1; MAP2K1; NFKB1; PAK3; ITGB3; |
| | CCND1; GSK3A; FRAP1; SFN; ITGA2; TTK; CSNK1A1; BRAF; GSK3B; |
| | AKT3; FOXO1; SGK; HSP90AA1; RPS6KB1 |
| ERK/MAPK Signaling | PRKCE; ITGAM; ITGA5; HSPB1; IRAK1; PRKAA2; EIF2AK2; RAC1; |
| | RAP1A; TLN1; EIF4E; ELK1; GRK6; MAPK1; RAC2; PLK1; AKT2; |
| | PIK3CA; CDK8; CREB1; PRKCI; PTK2; FOS; RPS6KA4; PIK3CB; |
| | PPP2R1A; PIK3C3; MAPK8; MAPK3; ITGA1; ETS1; KRAS; MYCN; |
| | EIF4EBP1; PPARG; PRKCD; PRKAA1; MAPK9; SRC; CDK2; PPP2CA; |
| | PIM1; PIK3C2A; ITGB7; YWHAZ; PPP1CC; KSR1; PXN; RAF1; FYN; |
| | DYRK1A; ITGB1; MAP2K2; PAK4; PIK3R1; STAT3; PPP2R5C; MAP2K1; |
| | PAK3; ITGB3; ESR1; ITGA2; MYC; TTK; CSNK1A1; CRKL; BRAF; ATF4; |
| | PRKCA; SRF; STAT1; SGK |
| Glucocorticoid | RAC1; TAF4B; EP300; SMAD2; TRAF6; PCAF; ELK1; MAPK1; SMAD3; |
| Receptor Signaling | AKT2; IKBKB; NCOR2; UBE2I; PIK3CA; CREB1; FOS; HSPA5; NFKB2; |
| | BCL2; MAP3K14; STAT5B; PIK3CB; PIK3C3; MAPK8; BCL2L1; MAPK3; |
| | TSC22D3; MAPK10; NRIP1; KRAS; MAPK13; RELA; STAT5A; MAPK9; |
| | NOS2A; PBX1; NR3C1; PIK3C2A; CDKN1C; TRAF2; SERPINE1; NCOA3; |
| | MAPK14; TNF; RAF1; IKBKG; MAP3K7; CREBBP; CDKN1A; MAP2K2; |
| | JAK1; IL8; NCOA2; AKT1; JAK2; PIK3R1; CHUK; STAT3; MAP2K1; |
| | NFKB1; TGFBR1; ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; IGF1; RAC1; RAP1A; |
| Signaling | E1F4E; PRKCZ; NRP1; NTRK2; ARHGEF7; SMO; ROCK2; MAPK1; PGF; |
| | RAC2; PTPN11; GNAS; AKT2; PIK3CA; ERBB2; PRKCI; PTK2; CFL1; |
| | GNAQ; PIK3CB; CXCL12; PIK3C3; WNT11; PRKD1; GNB2L1; ABL1; |
| | MAPK3; ITGA1; KRAS; RHOA; PRKCD; PIK3C2A; ITGB7; GLI2; PXN; |
| | VASP; RAF1; FYN; ITGB1; MAP2K2; PAK4; ADAM17; AKT1; PIK3R1; |
| | GLI1; WNT5A; ADAM10; MAP2K1; PAK3; ITGB3; CDC42; VEGFA; |
| | ITGA2; EPHA8; CRKL; RND1; GSK3B; AKT3; PRKCA |
| Ephrin Receptor | PRKCE; ITGAM; ROCK1; ITGA5; CXCR4; IRAK1; PRKAA2; EIF2AK2; |
| Signaling | RAC1; RAP1A; GRK6; ROCK2; MAPK1; PGF; RAC2; PTPN11; GNAS; |
| | PLK1; AKT2; DOK1; CDK8; CREB1; PTK2; CFL1; GNAQ; MAP3K14; |
| | CXCL12; MAPK8; GNB2L1; ABL1; MAPK3; ITGA1; KRAS; RHOA; |
| | PRKCD; PRKAA1; MAPK9; SRC; CDK2; PIM1; ITGB7; PXN; RAF1; FYN; |
| | DYRK1A; ITGB1; MAP2K2; PAK4, AKT1; JAK2; STAT3; ADAM10; |
| | MAP2K1; PAK3; ITGB3; CDC42; VEGFA; ITGA2; EPHA8; TTK; CSNK1A1; |
| | CRKL; BRAF; PTPN13; ATF4; AKT3; SGK |
| Actin Cytoskeleton | ACTN4; PRKCE; ITGAM; ROCK1; ITGA5; IRAK1; PRKAA2; EIF2AK2; |
| Signaling | RAC1; INS; ARHGEF7; GRK6; ROCK2; MAPK1; RAC2; PLK1; AKT2; |
| | PIK3CA; CDK8; PTK2; CFL1; PIK3CB; MYH9; DIAPH1; PIK3C3; MAPK8; |
| | F2R; MAPK3; SLC9A1; ITGA1; KRAS; RHOA; PRKCD; PRKAA1; MAPK9; |
| | CDK2; PIM1; PIK3C2A; ITGB7; PPP1CC; PXN; VIL2; RAF1; GSN; |
| | DYRK1A; ITGB1; MAP2K2; PAK4; PIP5K1A; PIK3R1; MAP2K1; PAK3; |
| | ITGB3; CDC42; APC; ITGA2; TTK; CSNK1A1; CRKL; BRAF; VAV3; SGK |
| Huntington's Disease | PRKCE; IGF1; EP300; RCOR1; PRKCZ; HDAC4; TGM2; MAPK1; CAPNS1; |
| Signaling | AKT2; EGFR; NCOR2; SP1; CAPN2; PIK3CA; HDAC5; CREB1; PRKC1; |
| | HSPA5; REST; GNAQ; PIK3CB; PIK3C3; MAPK8; IGF1R; PRKD1; |
| | GNB2L1; BCL2L1; CAPN1; MAPK3; CASP8; HDAC2; HDAC7A; PRKCD; |
| | HDAC11; MAPK9; HDAC9; PIK3C2A; HDAC3; TP53; CASP9; CREBBP; |
| | AKT1; PIK3R1; PDPK1; CASP1; APAF1; FRAP1; CASP2; JUN; BAX; ATF4; |
| | AKT3; PRKCA; CLTC; SGK; HDAC6; CASP3 |
| Apoptosis Signaling | PRKCE; ROCK1; BID; IRAK1; PRKAA2; EIF2AK2; BAK1; BIRC4; GRK6; |
| | MAPK1; CAPNS1; PLK1; AKT2; IKBKB; CAPN2; CDK8; FAS; NFKB2; |
| | BCL2; MAP3K14; MAPK8; BCL2L1; CAPN1; MAPK3; CASP8; KRAS; |
| | RELA; PRKCD; PRKAA1; MAPK9; CDK2; PIM1; TP53; TNF; RAF1; |
| | IKBKG; RELB; CASP9; DYRK1A; MAP2K2; CHUK; APAF1; MAP2K1; |
| | NFKB1; PAK3; LMNA; CASP2; BIRC2; TTK; CSNK1A1; BRAF; BAX; |
| | PRKCA; SGK; CASP3; BIRC3; PARP1 |
| B Cell Receptor | RAC1; PTEN; LYN; ELK1; MAPK1; RAC2; PTPN11; AKT2; IKBKB; |
| Signaling | PIK3CA; CREB1; SYK; NFKB2; CAMK2A; MAP3K14; PIK3CB; PIK3C3; |
| | MAPK8; BCL2L1; ABL1; MAPK3; ETS1; KRAS; MAPK13; RELA; PTPN6; |
| | MAPK9; EGR1; PIK3C2A; BTK; MAPK14; RAF1; IKBKG; RELB; MAP3K7; |
| | MAP2K2; AKT1; PIK3R1; CHUK; MAP2K1; NFKB1; CDC42; GSK3A; |
| | FRAP1; BCL6; BCL10; JUN; GSK3B; ATF4; AKT3; VAV3; RPS6KB1 |
| Leukocyte | ACTN4; CD44; PRKCE; ITGAM; ROCK1; CXCR4; CYBA; RAC1; RAP1A; |
| Extravasation Signaling | PRKCZ; ROCK2; RAC2; PTPN11; MMP14; PIK3CA; PRKCI; PTK2; |
| | PIK3CB; CXCL12; PIK3C3; MAPK8; PRKD1; ABL1; MAPK10; CYBB; |
| | MAPK13; RHOA; PRKCD; MAPK9; SRC; PIK3C2A; BTK; MAPK14; NOX1; |
| | PXN; VIL2; VASP; ITGB1; MAP2K2; CTNND1; PIK3R1; CTNNB1; CLDN1; |
| | CDC42; F11R; ITK; CRKL; VAV3; CTTN; PRKCA; MMP1; MMP9 |
| Integrin Signaling | ACTN4; ITGAM; ROCK1; ITGA5; RAC1; PTEN; RAP1A; TLN1; ARHGEF7; |
| | MAPK1; RAC2; CAPNS1; AKT2; CAPN2; PIK3CA; PTK2; PIK3CB; PIK3C3; |
| | MAPK8; CAV1; CAPN1; ABL1; MAPK3; ITGA1; KRAS; RHOA; SRC; |
| | PIK3C2A; ITGB7; PPP1CC; ILK; PXN; VASP; RAF1; FYN; ITGB1; |
| | MAP2K2; PAK4; AKT1; PIK3R1; TNK2; MAP2K1; PAK3; ITGB3; CDC42; |
| | RND3; ITGA2; CRKL; BRAF; GSK3B; AKT3 |
| Acute Phase Response | IRAK1; SOD2; MYD88; TRAF6; ELK1; MAPK1; PTPN11; AKT2; IKBKB; |
| Signaling | PIK3CA; FOS; NFKB2; MAP3K14; PIK3CB; MAPK8; RIPK1; MAPK3; |
| | IL6ST; KRAS; MAPK13; IL6R; RELA; SOCS1; MAPK9; FTL; NR3C1; |
| | TRAF2; SERPINE1; MAPK14; TNF; RAF1; PDK1; IKBKG; RELB; MAP3K7; |
| | MAP2K2; AKT1; JAK2; PIK3R1; CHUK; STAT3; MAP2K1; NFKB1; FRAP1; |
| | CEBPB; JUN; AKT3; IL1R1; IL6 |
| PTEN Signaling | ITGAM; ITGA5; RAC1; PTEN; PRKCZ; BCL2L11; MAPK1; RAC2; AKT2; |
| | EGFR; IKBKB; CBL; PIK3CA; CDKN1B; PTK2; NFKB2; BCL2; PIK3CB; |
| | BCL2L1; MAPK3; ITGA1; KRAS; ITGB7; ILK; PDGFRB; INSR; RAF1; |
| | IKBKG; CASP9; CDKN1A; ITGB1; MAP2K2; AKT1; PIK3R1; CHUK; |
| | PDGFRA; PDPK1; MAP2K1; NFKB1; ITGB3; CDC42; CCND1; GSK3A; |
| | ITGA2; GSK3B; AKT3; FOXO1; CASP3; RPS6KB1 |
| p53 Signaling | PTEN; EP300; BBC3; PCAF; FASN; BRCA1; GADD45A; BIRC5; AKT2; |
| | PIK3CA; CHEK1; TP53INP1; BCL2; PIK3CB; PIK3C3; MAPK8; THBS1; |
| | ATR; BCL2L1; E2F1; PMAIP1; CHEK2; TNFRSF10B; TP73; RB1; HDAC9; |
| | CDK2; PIK3C2A; MAPK14; TP53; LRDD; CDKN1A; HIPK2; AKT1; |
| | PIK3R1; RRM2B; APAF1; CTNNB1; SIRT1; CCND1; PRKDC; ATM; SFN; |
| | CDKN2A; JUN; SNAI2; GSK3B; BAχ; AKT3 |
| Aryl Hydrocarbon | HSPB1; EP300; FASN; TGM2; RXRA; MAPK1; NQO1; NCOR2; SP1; ARNT; |
| Receptor Signaling | CDKN1B; FOS; CHEK1; SMARCA4; NFKB2; MAPK8; ALDH1A1; ATR; |
| | E2F1; MAPK3; NRIP1; CHEK2; RELA; TP73; GSTP1; RB1; SRC; CDK2; |
| | AHR; NFE2L2; NCOA3; TP53; TNF; CDKN1A; NCOA2; APAF1; NFKB1; |
| | CCND1; ATM; ESR1; CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; NCOR2; PIK3CA; ARNT; |
| Signaling | PRKCI; NFKB2; CAMK2A; PIK3CB; PPP2R1A; PIK3C3; MAPK8; PRKD1; |
| | ALDH1A1; MAPK3; NRIP1; KRAS; MAPK13; PRKCD; GSTP1; MAPK9; |
| | NOS2A; ABCB1; AHR; PPP2CA; FTL; NFE2L2; PIK3C2A; PPARGC1A; |
| | MAPK14; TNF; RAF1; CREBBP; MAP2K2; PIK3R1; PPP2R5C; MAP2K1; |
| | NFKB1; KEAP1; PRKCA; EIF2AK3; IL6; CYP1B1; HSP90AA1 |
| SAPK/JNK Signaling | PRKCE; IRAK1; PRKAA2; EIF2AK2; RAC1; ELK1; GRK6; MAPK1; |
| | GADD45A; RAC2; PLK1; AKT2; PIK3CA; FADD; CDK8; PIK3CB; PIK3C3; |
| | MAPK8; RIPK1; GNB2L1; IRS1; MAPK3; MAPK10; DAXX; KRAS; |
| | PRKCD; PRKAA1; MAPK9; CDK2; PIM1; PIK3C2A; TRAF2; TP53; LCK; |
| | MAP3K7; DYRK1A; MAP2K2; PIK3R1; MAP2K1; PAK3; CDC42; JUN; |
| | TTK; CSNK1A1; CRKL; BRAF; SGK |
| PPAr/RXR Signaling | PRKAA2; EP300; INS; SMAD2; TRAF6; PPARA; FASN; RXRA; MAPK1; |
| | SMAD3; GNAS; IKBKB; NCOR2; ABCA1; GNAQ; NFKB2; MAP3K14; |
| | STAT5B; MAPK8; IRS1; MAPK3; KRAS; RELA; PRKAA1; PPARGC1A; |
| | NCOA3; MAPK14; INSR; RAF1; IKBKG; RELB; MAP3K7; CREBBP; |
| | MAP2K2; JAK2; CHUK; MAP2K1; NFKB1; TGFBR1; SMAD4; JUN; IL1R1; |
| | PRKCA; IL6; HSP90AA1; ADIPOQ |
| NF-KB Signaling | IRAK1; EIF2AK2; EP300; INS; MYD88; PRKCZ: TRAF6; TBK1; AKT2; |
| | EGFR; IKBKB; PIK3CA; BTRC; NFKB2; MAP3K14; PIK3CB; PIK3C3; |
| | MAPK8; RIPK1; HDAC2; KRAS; RELA; PIK3C2A; TRAF2; TLR4: |
| | PDGFRB; TNF; INSR; LCK; IKBKG; RELB; MAP3K7; CREBBP; AKT1; |
| | PIK3R1; CHUK; PDGFRA; NFKB1; TLR2; BCL10; GSK3B; AKT3; |
| | TNFAIP3; IL1R1 |
| Neuregulin Signaling | ERBB4; PRKCE; ITGAM; ITGA5: PTEN; PRKCZ; ELK1; MAPK1; PTPN11; |
| | AKT2; EGFR; ERBB2; PRKCI; CDKN1B; STAT5B; PRKD1; MAPK3; |
| | ITGA1; KRAS; PRKCD; STAT5A; SRC; ITGB7; RAF1; ITGB1; MAP2K2; |
| | ADAM17; AKT1; PIK3R1; PDPK1; MAP2K1; ITGB3; EREG; FRAP1; |
| | PSEN1; ITGA2; MYC; NRG1; CRKL; AKT3; PRKCA; HSP90AA1; RPS6KB1 |
| Wnt & Beta catenin | CD44; EP300; LRP6; DVL3; CSNK1E; GJA1; SMO; AKT2; PIN1; CDH1; |
| Signaling | BTRC; GNAQ; MARK2; PPP2R1A; WNT11; SRC; DKK1; PPP2CA; SOX6; |
| | SFRP2: ILK; LEF1; SOX9; TP53; MAP3K7; CREBBP; TCF7L2; AKT1; |
| | PPP2R5C; WNT5A; LRP5; CTNNB1; TGFBR1; CCND1; GSK3A; DVL1; |
| | APC; CDKN2A; MYC; CSNK1A1; GSK3B; AKT3; SOX2 |
| Insulin Receptor | PTEN; INS; EIF4E; PTPN1; PRKCZ; MAPK1; TSC1; PTPN11; AKT2; CBL; |
| Signaling | PIK3CA; PRKCI; PIK3CB; PIK3C3; MAPK8; IRS1; MAPK3; TSC2; KRAS; |
| | EIF4EBP1; SLC2A4; PIK3C2A; PPP1CC; INSR; RAF1; FYN; MAP2K2; |
| | JAK1; AKT1; JAK2; PIK3R1; PDPK1; MAP2K1; GSK3A; FRAP1; CRKL; |
| | GSK3B; AKT3; FOXO1; SGK; RPS6KB1 |
| IL-6 Signaling | HSPB1; TRAF6; MAPKAPK2; ELK1; MAPK1; PTPN11; IKBKB; FOS; |
| | NFKB2: MAP3K14; MAPK8; MAPK3; MAPK10; IL6ST; KRAS; MAPK13; |
| | IL6R; RELA; SOCS1; MAPK9; ABCB1; TRAF2; MAPK14; TNF; RAF1; |
| | IKBKG; RELB; MAP3K7; MAP2K2; IL8; JAK2; CHUK; STAT3; MAP2K1; |
| | NFKB1; CEBPB; JUN; IL1R1; SRF; IL6 |
| Hepatic Cholestasis | PRKCE; IRAK1; INS; MYD88; PRKCZ; TRAF6; PPARA; RXRA; IKBKB; |
| | PRKCI; NFKB2; MAP3K14; MAPK8; PRKD1; MAPK10; RELA; PRKCD; |
| | MAPK9; ABCB1; TRAF2; TLR4; TNF; INSR; IKBKG; RELB; MAP3K7; IL8; |
| | CHUK; NR1H2; TJP2; NFKB1; ESR1; SREBF1; FGFR4; JUN; IL1R1; |
| | PRKCA; IL6 |
| IGF-1 Signaling | IGF1; PRKCZ; ELK1; MAPK1; PTPN11; NEDD4; AKT2; PIK3CA; PRKCI; |
| | PTK2; FOS; PIK3CB; PIK3C3; MAPK8; IGF1R; IRS1; MAPK3; IGFBP7; |
| | KRAS; PIK3C2A; YWHAZ; PXN; RAF1; CASP9; MAP2K2; AKT1; PIK3R1; |
| | PDPK1; MAP2K1; IGFBP2; SFN; JUN; CYR61; AKT3; FOXO1; SRF; CTGF; |
| | RPS6KB1 |
| NRF2-mediated | PRKCE; EP300; SOD2; PRKCZ; MAPK1; SQSTM1; NQO1; PIK3CA; PRKCI; |
| Oxidative Stress | FOS; PIK3CB; PIK3C3; MAPK8; PRKD1; MAPK3; KRAS; PRKCD; GSTP1; |
| Response | MAPK9; FTL; NFE2L2; PIK3C2A; MAPK14; RAF1; MAP3K7; CREBBP; |
| | MAP2K2; AKT1; PIK3R1; MAP2K1; PPIB; JUN; KEAP1; GSK3B; ATF4; |
| | PRKCA; EIF2AK3; HSP90AA1 |
| Hepatic | EDN1; IGF1; KDR; FLT1; SMAD2; FGFR1; MET; PGF; SMAD3; EGFR; |
| Fibrosis/Hepatic | FAS; CSF1; NFKB2; BCL2; MYH9; IGF1R; IL6R; RELA; TLR4; PDGFRB; |
| Stellate Cell Activation | TNF; RELB; IL8; PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; NCOR2; FOS; |
| | NFKB2; MAP3K14; STAT5B; MAPK3; NRIP1; KRAS; PPARG; RELA; |
| | STAT5A; TRAF2; PPARGC1A; PDGFRB; TNF; INSR; RAF1; IKBKG; |
| | RELB; MAP3K7; CREBBP; MAP2K2; CHUK; PDGFRA; MAP2K1; NFKB1; |
| | JUN; IL1R1; HSP90AA1 |
| Fc Epsilon RI Signaling | PRKCE; RAC1; PRKCZ; LYN; MAPK1; RAC2; PTPN11; AKT2; PIK3CA; |
| | SYK; PRKCI; PIK3CB; PIK3C3; MAPK8; PRKD1; MAPK3; MAPK10; |
| | KRAS; MAPK13; PRKCD; MAPK9; PIK3C2A; BTK; MAPK14; TNF; RAF1; |
| | FYN; MAP2K2; AKT1; PIK3R1; PDPK1; MAP2K1; AKT3; VAV3; PRKCA |
| G-Protein Coupled | PRKCE; RAP1A; RGS16; MAPK1; GNAS; AKT2; IKBKB; PIK3CA; CREB1; |
| Receptor Signaling | GNAQ; NFKB2; CAMK2A; PIK3CB; PIK3C3; MAPK3; KRAS; RELA; SRC; |
| | PIK3C2A; RAF1; IKBKG; RELB; FYN; MAP2K2; AKT1; PIK3R1; CHUK; |
| | PDPK1; STAT3; MAP2K1; NFKB1; BRAF; ATF4; AKT3; PRKCA |
| Inositol Phosphate | PRKCE; IRAK1; PRKAA2; EIF2AK2; PTEN; GRK6; MAPK1; PLK1; AKT2; |
| Metabolism | PIK3CA; CDK8; PIK3CB; PIK3C3; MAPK8; MAPK3; PRKCD; PRKAA1; |
| | MAPK9; CDK2; PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; PIK3C3; MAPK8; |
| | CAV1; ABL1; MAPK3; KRAS; SRC; PIK3C2A; PDGFRB; RAF1; MAP2K2; |
| | JAK1; JAK2; PIK3R1; PDGFRA; STAT3; SPHK1; MAP2K1; MYC; JUN; |
| | CRKL; PRKCA; SRF; STAT1; SPHK2 |
| VEGF Signaling | ACTN4; ROCK1; KDR; FLT1; ROCK2; MAPK1; PGF; AKT2; PIK3CA; |
| | ARNT; PTK2; BCL2; PIK3CB; PIK3C3; BCL2L1; MAPK3; KRAS; HIF1A; |
| | NOS3; PIK3C2A; PXN; RAF1; MAP2K2; ELAVL1; AKT1; PIK3R1; |
| | MAP2K1; SFN; VEGFA; AKT3; FOXO1; PRKCA |
| Natural Killer Cell | PRKCE; RAC1; PRKCZ; MAPK1; RAC2; PTPN11; KIR2DL3; AKT2; |
| Signaling | PIK3CA; SYK; PRKCI; PIK3CB; PIK3C3; PRKD1; MAPK3; KRAS; PRKCD; |
| | PTPN6; PIK3C2A; LCK; RAF1; FYN; MAP2K2; PAK4; AKT1; PIK3R1; |
| | MAP2K1; PAK3; AKT3; VAV3; PRKCA |
| Cell Cycle: G1/S | HDAC4; SMAD3; SUV39H1; HDAC5; CDKN1B; BTRC; ATR; ABL1; E2F1; |
| Checkpoint Regulation | HDAC2; HDAC7A; RB1; HDAC11; HDAC9; CDK2; E2F2; HDAC3; TP53; |
| | CDKN1A; CCND1; E2F4; ATM; RBL2; SMAD4; CDKN2A; MYC; NRG1; |
| | GSK3B; RBL1; HDAC6 |
| T Cell Receptor | RAC1; ELK1; MAPK1; IKBKB; CBL; PIK3CA; FOS; NFKB2; PIK3CB; |
| Signaling | PIK3C3; MAPK8; MAPK3; KRAS; RELA, PIK3C2A; BTK; LCK; RAF1; |
| | IKBKG; RELB, FYN; MAP2K2; PIK3R1; CHUK; MAP2K1; NFKB1; ITK; |
| | BCL10; JUN; VAV3 |
| Death Receptor | CRADD; HSPB1; BID; BIRC4; TBK1; IKBKB; FADD; FAS; NFKB2; BCL2; |
| Signaling | MAP3K14; MAPK8; RIPK1; CASP8; DAXX; TNFRSF10B; RELA; TRAF2; |
| | TNF; IKBKG; RELB; CASP9; CHUK; APAF1; NFKB1; CASP2; BIRC2; |
| | CASP3; BIRC3 |
| FGF Signaling | RAC1; FGFR1; MET; MAPKAPK2; MAPK1; PTPN11; AKT2; PIK3CA; |
| | CREB1; PIK3CB; PIK3C3; MAPK8; MAPK3; MAPK13; PTPN6; PIK3C2A; |
| | MAPK14; RAF1; AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; STAT5B; |
| | PIK3CB; PIK3C3; GNB2L1; BCL2L1; MAPK3; ETS1; KRAS; RUNX1; PIM1; |
| | PIK3C2A; RAF1; MAP2K2; AKT1; JAK2; PIK3R1; STAT3; MAP2K1; |
| | CCND1; AKT3; STAT1 |
| Amyotrophic Lateral | BID; IGF1; RAC1; BIRC4; PGF; CAPNS1; CAPN2; PIK3CA; BCL2; PIK3CB; |
| Sclerosis Signaling | PIK3C3; BCL2L1; CAPN1; PIK3C2A; TP53; CASP9; PIK3R1; RAB5A; |
| | CASP1; APAF1; VEGFA; BIRC2; BAχ; AKT3; CASP3; BIRC3 |
| JAK/Stat Signaling | PTPN1; MAPK1; PTPN11; AKT2; PIK3CA; STAT5B; PIK3CB; PIK3C3; |
| | MAPK3; KRAS; SOCS1; STAT5A; PTPN6; PIK3C2A; RAF1; CDKN1A; |
| | MAP2K2; JAK1; AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; PLK1; AKT2; CDK8; |
| Nicotinamide | MAPK8; MAPK3; PRKCD; PRKAA1; PBEF1; MAPK9; CDK2; PIM1; |
| Metabolism | DYRK1A; MAP2K2; MAP2K1; PAK3; NT5E; TTK; CSNK1A1; BRAF; SGK |
| Chemokine Signaling | CXCR4; ROCK2; MAPK1; PTK2; FOS; CFL1; GNAQ; CAMK2A; CXCL12; |
| | MAPK8; MAPK3; KRAS; MAPK13; RHOA; CCR3; SRC; PPP1CC; MAPK14; |
| | NOX1; RAF1; MAP2K2; MAP2K1; JUN; CCL2; PRKCA |
| IL-2 Signaling | ELK1; MAPK1; PTPN11; AKT2; PIK3CA; SYK; FOS; STAT5B; PIK3CB; |
| | PIK3C3; MAPK8; MAPK3; KRAS; SOCS1; STAT5A; PIK3C2A; LCK; RAF1; |
| | MAP2K2; JAK1; AKT1; PIK3R1; MAP2K1; JUN; AKT3 |
| Synaptic Long Term | PRKCE; IGF1; PRKCZ; PRDX6; LYN; MAPK1; GNAS; PRKCI; GNAQ; |
| Depression | PPP2R1A; IGF1R; PRKD1; MAPK3; KRAS; GRN; PRKCD; NOS3; NOS2A; |
| | PPP2CA; YWHAZ; RAF1; MAP2K2; PPP2R5C; MAP2K1; PRKCA |
| Estrogen Receptor | TAF4B; EP300; CARM1; PCAF; MAPK1; NCOR2; SMARCA4; MAPK3; |
| Signaling | NRIP1; KRAS; SRC; NR3C1; HDAC3; PPARGC1A; RBM9; NCOA3; RAF1; |
| | CREBBP; MAP2K2; NCOA2; MAP2K1; PRKDC; ESR1; ESR2 |
| Protein Ubiquitination | TRAF6; SMURF1; BIRC4; BRCA1; UCHL1; NEDD4; CBL; UBE2I; BTRC; |
| Pathway | HSPA5; USP7; USP10; FBXW7; USP9X; STUB1; USP22; B2M; BIRC2; |
| | PARK2; USP8; USP1; VHL; HSP90AA1; BIRC3 |
| IL-10 Signaling | TRAF6; CCR1; ELK1; IKBKB; SP1; FOS; NFKB2; MAP3K14; MAPK8; |
| | MAPK13; RELA; MAPK14; TNF; IKBKG; RELB; MAP3K7; JAK1; CHUK; |
| | STAT3; NFKB1; JUN; IL1R1; IL6 |
| VDR/RXR Activation | PRKCE; EP300; PRKCZ; RXRA; GADD45A; HES1; NCOR2; SP1; PRKCI; |
| | CDKN1B; PRKD1; PRKCD; RUNX2; KLF4; YY1; NCOA3; CDKN1A; |
| | NCOA2; SPP1; LRP5; CEBPB; FOXO1; PRKCA |
| TGF-beta Signaling | EP300; SMAD2; SMURF1; MAPK1; SMAD3; SMAD1; FOS; MAPK8; |
| | MAPK3; KRAS; MAPK9; RUNX2; SERPINE1; RAF1; MAP3K7; CREBBP; |
| | MAP2K2; MAP2K1; TGFBR1; SMAD4; JUN; SMAD5 |
| Toll-like Receptor | IRAK1; EIF2AK2; MYD88; TRAF6; PPARA; ELK1; IKBKB; FOS; NFKB2; |
| Signaling | MAP3K14; MAPK8; MAPK13; RELA; TLR4; MAPK14; IKBKG; RELB; |
| | MAP3K7; CHUK; NFKB1; TLR2; JUN |
| p38 MAPK Signaling | HSPB1; IRAK1; TRAF6; MAPKAPK2; ELK1; FADD; FAS; CREB1; DDIT3; |
| | RPS6KA4; DAXX; MAPK13; TRAF2; MAPK14; TNF; MAP3K7; TGFBR1; |
| | MYC; ATF4; IL1R1; SRF; STAT1 |
| Neurotrophin/TRK | NTRK2; MAPK1; PTPN11; PIK3CA; CREB1; FOS; PIK3CB; PIK3C3; |
| Signaling | MAPK8; MAPK3; KRAS; PIK3C2A; RAF1; MAP2K2; AKT1; PIK3R1; |
| | PDPK1; MAP2K1; CDC42; JUN; ATF4 |
| FXR/RXR Activation | INS; PPARA; FASN; RXRA; AKT2; SDC1; MAPK8; APOB; MAPK10; |
| | PPARG; MTTP; MAPK9; PPARGC1A; TNF; CREBBP; AKT1; SREBF1; |
| | FGFR4; AKT3; FOXO1 |
| Synaptic Long Term | PRKCE; RAP1A; EP300; PRKCZ; MAPK1; CREB1; PRKCI; GNAQ; |
| Potentiation | CAMK2A; PRKD1; MAPK3; KRAS; PRKCD; PPP1CC; RAF1; CREBBP; |
| | MAP2K2; MAP2K1; ATF4; PRKCA |
| Calcium Signaling | RAP1A; EP300; HDAC4; MAPK1; HDAC5; CREB1; CAMK2A; MYH9; |
| | MAPK3; HDAC2; HDAC7A; HDAC11; HDAC9; HDAC3; CREBBP; CALR; |
| | CAMKK2; ATF4; HDAC6 |
| EGF Signaling | ELK1; MAPK1; EGFR; PIK3CA; FOS; PIK3CB; PIK3C3; MAPK8; MAPK3; |
| | PIK3C2A; RAF1; JAK1; PIK3R1; STAT3; MAP2K1; JUN; PRKCA; SRF; |
| | STAT1 |
| Hypoxia Signaling in | EDN1; PTEN; EP300; NQO1; UBE2I; CREB1; ARNT; HIF1A; SLC2A4; |
| the Cardiovascular | NOS3; TP53; LDHA; AKT1; ATM; VEGFA; JUN; ATF4; VHL; HSP90AA1 |
| System | |
| LPS/IL-1 Mediated | IRAK1; MYD88; TRAF6; PPARA; RXRA; ABCA1, MAPK8; ALDH1A1; |
| Inhibition of RXR | GSTP1; MAPK9; ABCB1; TRAF2; TLR4; TNF; MAP3K7; NR1H2; SREBF1; |
| Function | JUN; IL1R1 |
| LXR/RXR Activation | FASN; RXRA; NCOR2; ABCA1; NFKB2; IRF3; RELA; NOS2A; TLR4; TNF; |
| | RELB; LDLR; NR1H2; NFKB1; SREBF1; IL1R1; CCL2; IL6; MMP9 |
| Amyloid Processing | PRKCE; CSNK1E; MAPK1; CAPNS1; AKT2; CAPN2; CAPN1; MAPK3; |
| | MAPK13; MAPT; MAPK14; AKT1; PSEN1; CSNK1A1; GSK3B; AKT3; APP |
| IL-4 Signaling | AKT2; PIK3CA; PIK3CB; PIK3C3; IRS1; KRAS; SOCS1; PTPN6; NR3C1; |
| | PIK3C2A; JAK1; AKT1; JAK2; PIK3R1; FRAP1; AKT3; RPS6KB1 |
| Cell Cycle: G2/M DNA | EP300; PCAF; BRCA1; GADD45A; PLK1; BTRC; CHEK1; ATR; CHEK2; |
| Damage Checkpoint | YWHAZ; TP53; CDKN1A; PRKDC; ATM; SFN; CDKN2A |
| Regulation | |
| Nitric Oxide Signaling | KDR; FLT1; PGF; AKT2; PIK3CA; PIK3CB; PIK3C3; CAV1; PRKCD; NOS3; |
| in the Cardiovascular | PIK3C2A; AKT1; PIK3R1; VEGFA; AKT3; HSP90AA1 |
| System | |
| Purine Metabolism | NME2; SMARCA4; MYH9; RRM2; ADAR; EIF2AK4; PKM2; ENTPD1; |
| | RAD51; RRM2B; TJP2; RAD51C; NT5E; POLD1; NME1 |
| cAMP-mediated | RAP1A; MAPK1; GNAS; CREB1; CAMK2A; MAPK3; SRC; RAF1; |
| Signaling | MAP2K2; STAT3; MAP2K1; BRAF; ATF4 |
| Mitochondrial | SOD2; MAPK8; CASP8; MAPK10; MAPK9; CASP9; PARK7; PSEN1; |
| Dysfunction | PARK2; APP; CASP3 |
| Notch Signaling | HES1; JAG1; NUMB; NOTCH4; ADAM17; NOTCH2; PSEN1; NOTCH3; |
| | NOTCH1; DLL4 |
| Endoplasmic Reticulum | HSPA5; MAPK8; XBP1; TRAF2; ATF6; CASP9; ATF4; EIF2AK3; CASP3 |
| Stress Pathway | |
| Pyrimidine Metabolism | NME2; AICDA; RRM2; EIF2AK4; ENTPD1; RRM2B; NT5E; POLD1; NME1 |
| Parkinson's Signaling | UCHL1; MAPK8; MAPK13; MAPK14; CASP9; PARK7; PARK2; CASP3 |
| Cardiac & Beta | GNAS; GNAQ; PPP2R1A; GNB2L1; PPP2CA; PPP1CC; PPP2R5C |
| Adrenergic Signaling | |
| Glycolysis/ | HK2; GCK; GPI; ALDH1A1; PKM2; LDHA; HK1 |
| Gluconeogenesis | |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRKIB |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars | NQO1; HK2; GCK; HK1 |
| Metabolism | |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione | IDH2; GSTP1; ANPEP; IDH1 |
| Metabolism | |
| Glycerolipid | ALDH1A1; GPAM; SPHK1; SPHK2 |
| Metabolism | |
| Linoleic Acid | PRDX6; GRN; YWHAZ; CYP1B1 |
| Metabolism | |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine | PRDX6; PRDX1; TYR |
| and Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics by | |
| Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid | SPHK1; SPHK2 |
| Metabolism | |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative Stress | |
| Response | |
| Pentose Phosphate | GPI |
| Pathway | |
| Pentose and | UCHL1 |
| Glucuronate | |
| Interconversions | |
| Retinol Metabolism | ALDH1A1 |
| Riboflavin Metabolism | TYR |
| Tyrosine Metabolism | PRMT5, TYR |
| Ubiquinone | PRMT5 |
| Biosynthesis | |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cm2; Grk2; Trpa1; Pomc; Cgrp; Crf; |
| | Pka; Era; Nr2b; TRPM5; Prkaca; Prkacb; Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; Wnt2b; Wnt3a; Wnt4; |
| Neurology | Wnt5a; Wnt6; Wnt7b; Wnt8b; Wnt9a; Wnt9b; Wnt10a; Wnt10b; Wnt16); beta- |
| | catenin; Dkk-1; Frizzled related proteins; Otx-2; Gbx2; FGF-8; Reelin; Dab1; |
| | unc-86 (Pou4fl or Brn3a); Numb; Reln |

### Vectors

There are provided, in some embodiments, vectors (e.g., viral vectors). The viral vector can be an RNA viral vector. The polynucleotide can be derived from a positive sense RNA virus, a negative sense RNA virus, an ambisense RNA virus, or any combination thereof. The polynucleotide can be derived from a single-stranded RNA virus. The polynucleotide can be derived from a negative-strand RNA virus. The polynucleotide can be derived from one or more negative-strand RNA viruses of the order Mononegavirales. The nucleoprotein (N), phosphoprotein (P), matrix protein (M), and/or RNA-dependent RNA polymerase (L) can be derived from one or more negative-strand RNA viruses of the order Mononegavirales (e.g., a bomaviridae virus, a filoviridae virus, a nyamiviridae virus, a paramyxodiridae virus, a rhabdoviridae virus, or any combination thereof). The Mononegavirales virus can comprise rabies virus, sendai virus, vesicular stomatitis virus, or any combination thereof. A Mononegavirales-based viral vector can comprise one or more attenuating mutations. In some embodiments, the one or more negative-strand RNA viruses of the order Mononegavirales can comprise an attenuated rabies virus strain (e.g., CVS-N2c, CVS-B2c, DRV-4, RRV-27, SRV-16, ERA, CVS-11, SAD B19, SPBN, SN-10, SN10-333, PM, LEP, SAD, or any combination thereof).

Viral vectors and methods of using are provided in PCT Application Publication No. WO2020/210655A1 and U.S. Patent Publication No. 2020/0165576, the content of each of which is incorporated herein by reference in its entirety. The viral vector can be modified so that the viral vector is targeted to a particular target environment of interest such as central nervous system, and to enhance tropism to the target environment of interest (e.g, CNS tropism). In some embodiments, the viral vector is AAV-CAP.B22. Broad gene expression throughout the mouse and marmoset brain after intravenous delivery of engineered AAV capsids has been described in Flytzanis et al. (“Broad gene expression throughout the mouse and marmoset brain after intravenous delivery of engineered AAV capsids” biorxiv, 2020), the content of which is incorporated herein by reference in its entirety.

Exemplary viral vectors that can be used in the methods, compositions, systems and kits described herein include those provided in US 20200071723A1, the content of which is incorporated herein by reference in its entirety. In some embodiments, the vector can comprise an adenovirus vector, an adeno-associated virus vector (AAV), an Epstein-Barr virus vector, a Herpes virus vector, an attenuated HIV vector, a retroviral vector, a vaccinia virus vector, or any combination thereof. In some embodiments, the vector can comprise an RNA viral vector. In some embodiments, the vector can be derived from one or more negative-strand RNA viruses of the order Mononegavirales. In some embodiments, the vector can be a rabies viral vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Retroviral vectors can be “defective”, i.e. unable to produce viral proteins required for productive infection. Replication of the vector can require growth in the packaging cell line. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

In some embodiment, the vectors can include a regulatory sequence that allows, for example, the translation of multiple proteins from a single mRNA. Non-limiting examples of such regulatory sequences include internal ribosome entry site (IRES) and 2A self-processing sequence. In some embodiments, the 2A sequence is a 2A peptide site from foot-and-mouth disease virus (F2A sequence). In some embodiments, the F2A sequence has a standard furin cleavage site. In some embodiments, the vector can also comprise regulatory control elements known to one of skill in the art to influence the expression of the RNA and/or protein products encoded by the polynucleotide within desired cells of the subject. In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an ration and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited, to the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide m response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

### Methods of Detecting and Monitoring

In some embodiments, the methods and compositions provided herein are useful in detecting a disease or disorder and/or monitoring the progression of a disease or disorder. As used herein, the term “diagnostic” refers identifying the presence or absence of or nature of a disease or disorder. Such detection methods can be used, for example, for early diagnosis of the condition, to determine whether a subject is predisposed to a disease or disorder, to monitor the progress of the disease or disorder or the progress of treatment protocols, to assess the severity of the disease or disorder, to forecast the an outcome of a disease or disorder and/or prospects of recovery, or to aid in the determination of a suitable treatment for a subject. The detection can occur in vitro or in vivo. The payload protein can comprise an imaging agent, such as, for example, a diagnostic agent. The payload protein can comprise a diagnostic contrast agent. The diagnostic agent can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

In some embodiments, the payload protein encodes a diagnostic agent. In some embodiments, the diagnostic agent aids in the identification of a unique cell type and/or a unique cell state. The diagnostic agent can be a molecule capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. For example, the diagnostic agent may comprise, in some embodiments, a fluorescent protein, such as, but not limited to, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, rsCherry, rsCherryRev, or any combination thereof. In some embodiments, the expression, stability, and/or activity (e.g., fluorescence) of the diagnostic agent is configured to be responsive to a disease state or a disorder state.

In some embodiments, the diagnostic agent aids in the identification of a unique cell type and/or a unique cell state. The unique cell type and/or a unique cell state can comprise lesions (e.g. tumors, infected cells). Detection and/or imaging of the diagnostic agent can enable a clinician to intraoperatively, laparoscopically, intravascularly or endoscopically detect said lesions. In some such embodiments, discrimination between lesions (e.g. tumors) and non-lesions (e.g. non-tumor tissue) is enhanced by the detection and/or imaging of the diagnostic agent. In some embodiments, detection and/or imaging of the diagnostic agent can enable a clinician to accurately locate lesions in a patient and thereby aid resection, irradiation, biopsy and/or lesion removal. In some embodiments, detection and/or imaging of the diagnostic agent aids the detection of non-malignant pathological lesions, such as, an infarct, including myocardial, atherosclerotic plaque, clot, including thrombosis, pulmonary embolism, infectious or inflammatory lesion, non-tumorous or noninfectious inflammation, or hyperplasia. The detection and/or imaging of the diagnostic agent may also be used to detect various stages of progression or severity of disease (e.g., benign, premalignant, and malignant breast lesions, tumor growth, or metastasis). The detection and/or imaging of the diagnostic agent may also be used to detect the response of the disease to prophylactic or therapeutic treatments or other interventions. The detection and/or imaging of the diagnostic agent can furthermore be used to help the medical practitioner in determining prognosis (e.g., worsening, status-quo, partial recovery, or complete recovery) of the patient, and the appropriate course of action. Detection and/or imaging of the diagnostic agent can be performed, for example, using an ultrasound scanner, a magnetic resonance imaging instrument (MRI scanner), an X-ray source with film or a detector (e.g., conventional or digital radiography system), an X-ray computed tomography (CT) or computed axial tomography (CAT) scanner, a gamma camera, or a positron emission tomography (PET) scanner.

### Pharmaceutical Compositions and Methods of Administration

Also disclosed herein are pharmaceutical compositions comprising one or more of the nucleic acids, vectors, and/or compositions provided herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioners.

The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween^{IM}, Pluronics™ or polyethylene glycol (PEG). In some embodiments, the physiologically acceptable carrier is an aqueous pH buffered solution.

Titers of vectors to be administered will vary depending, for example, on the particular viral vector, the mode of administration, the treatment goal, the individual, and the cell type(s) being targeted, and can be determined by methods standard in the art.

As will be readily apparent to one skilled in the art, the useful in vivo dosage of the nucleic acids, vectors, and/or compositions to be administered and the particular mode of administration will vary depending upon the age, weight, the severity of the affliction, and animal species treated, the particular IFFL that is used, and the specific use for which the IFFL is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. Dosages of nucleic acids, vectors, and/or compositions provided can depend primarily on factors such as the condition being treated, the age, weight and health of the patient, and may thus vary among patients. For example, a therapeutically effective human dosage of the viral vector is generally in the range of from about 0.1 ml to about 100 ml of solution containing concentrations of from about 1×10⁹ to 1×10¹⁶ genomes virus viral. A preferred human dosage can be about 1×10¹³ to 1×10¹⁶ viral vector genomes. The dosage will be adjusted to balance the therapeutic benefit against any side effects and such dosages may vary depending upon the therapeutic application for which the recombinant vector is employed. The levels of expression of the payload can be monitored to determine the amount and/or frequency of dosage resulting from the viral vector in some embodiments.

Nucleic acids, vectors, and/or compositions disclosed herein can be administered to a subject (e.g., a human) in need thereof. The route of the administration is not particularly limited. For example, a therapeutically effective amount of nucleic acids, vectors, and/or compositions can be administered to the subject by via routes standard in the art. Route(s) of administration can be readily determined by one skilled in the art taking into account the infection and/or disease state being treated and the target cells/tissue(s) that are to express the payload protein.

The administering can comprise systemic administration (e.g., intravenous, intramuscular, intraperitoneal, or intraarticular). Administering can comprise intrathecal administration, intracranial injection, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

Administering can comprise an injection into a brain region (e.g., direct administration to the brain parenchyma). The brain region can comprise the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kolliker-Fuse nucleus), Rostral interstitial nucleus of medial longitudinal fasciculus, Midbrain reticular formation, Dorsal raphe nucleus, Red nucleus, Ventral tegmental area, Substantianigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Comu ammonis area 1, Cornu ammonis area 2, Cornu ammonis area 3, Comu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (S1), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof. The brain region can comprise neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

Nucleic acids, vectors, and/or compositions to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of nucleic acids, vectors, and/or compositions expressing the therapeutic protein is administered to a host in need of such treatment. The use of the nucleic acids, vectors, and/or compositions provided herein in the manufacture of a medicament for inducing immunity in, or providing gene therapy to, a host is within the scope of the present application.

A therapeutically effective amount of the nucleic acids, vectors, and/or compositions provided herein can be administered to a subject at various points of time. For example, the nucleic acids, vectors, and/or compositions provided herein can be administered to the subject prior to, during, or after the subject has developed a disease, disorder, and/or infection. The nucleic acids, vectors, and/or compositions provided herein can also be administered to the subject prior to, during, or after the occurrence of a disease, disorder, and/or infection. In some embodiments, the nucleic acids, vectors, and/or compositions provided herein are administered to the subject during remission of the disease or disorder. In some embodiments, the nucleic acids, vectors, and/or compositions provided herein are administered prior to the onset of the disease or disorder in the subject. In some embodiments, nucleic acids, vectors, and/or compositions provided herein are administered to a subject at a risk of developing the disease or disorder.

The dosing frequency of the nucleic acids, vectors, and/or compositions provided herein can vary. For example, nucleic acids, vectors, and/or compositions provided herein can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the nucleic acids, vectors, and/or compositions provided herein are administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

### EXAMPLES

Some aspects of the embodiments discussed above are disclosed in further detail in the following examples, which are not in any way intended to limit the scope of the present disclosure.

### Example 1

miRNA Circuit Modules for Precise, Tunable Control of Gene Expression

The ability to express transgenes at specified levels is critical for understanding cellular behaviors, and for applications in gene and cell therapy. Transfection, viral vectors, and other gene delivery methods produce varying protein expression levels, with limited quantitative control, while targeted knock-in and stable selection are inefficient and slow. Active compensation mechanisms can improve precision, but the need for additional proteins or lack of tunability have prevented their widespread use. A toolkit of compact, synthetic miRNA-based circuit modules is disclosed herein that provides precise, tunable control of transgenes across diverse cell types. These circuits, termed DIMMERs (Dosage-Invariant miRNA-Mediated Expression Regulators), in some embodiments, and without being bound by any particular theory, use multivalent miRNA regulatory interactions within an incoherent feed-forward loop architecture to achieve nearly uniform protein expression over more than two orders of magnitude variation in underlying gene dosages or transcription rates. In some embodiments, they also allow coarse and fine control of expression, and are portable, functioning across diverse cell types. In addition, a heuristic miRNA design algorithm enables the creation of orthogonal circuit variants that independently control multiple genes in the same cell. These circuits allowed dramatically improved CRISPR imaging, and super-resolution imaging of EGFR receptors with transient transfections. The toolbox provided here can enable precise, tunable, dosage-invariant expression for research, gene therapy, and other biotechnology applications.

### Introduction

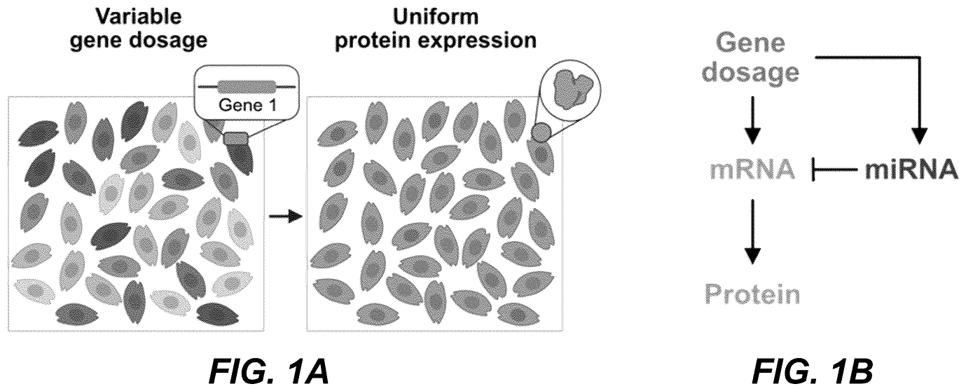

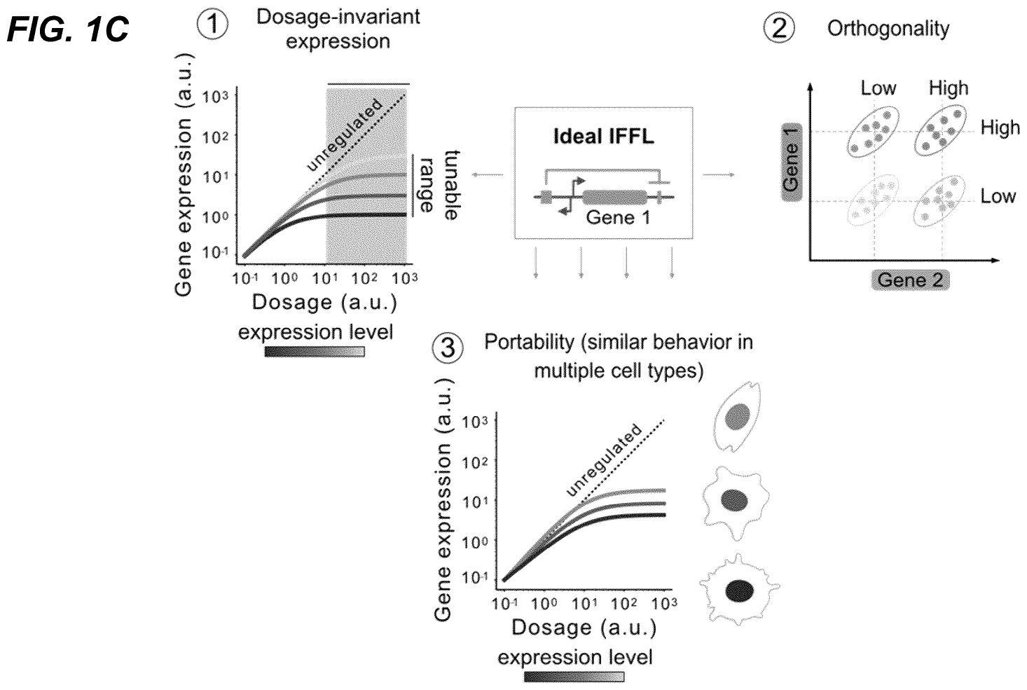

Biotechnology and biomedical research rely heavily on ectopic expression of transgenes in living cells. Popular expression systems produce a broad range of expression levels in individual cells. This is true for non-integrating approaches such as DNA transfection and AAV vectors, as well as integrating systems such as lentivirus or piggyBac transposons. This heterogeneity reflects unavoidable variation in the number of gene copies taken up, integrated, and expressed by each cell, as well as gene expression noise. Variability or noise may be tolerable or even useful in some situations, but more often presents an obstacle to accurate analysis and precise control of cell behaviors. Selecting for stable clones can reduce expression variation but is time-consuming, and can also be susceptible to stochastic silencing. An ideal gene regulation system would compensate for this variation, allowing more precise control of expression level, reduced toxicity in gene and cell therapies, and lower backgrounds with reporters, among other applications (FIG. 1A).

The incoherent feed-forward loop (IFFL) circuit motif provides an ideal mechanism to provide these capabilities. In an IFFL, a target gene and its negative regulator are co-regulated by the same input (FIG. 1B). Gene dosage can be considered such an input, and proportionately affects expression of both the regulator and its target. In some parameter regimes, these two effects cancel out, and target expression approaches a fixed level at high dosage (FIG. 1C, Supplementary Modeling). An ideal IFFL system should further allow tuning of this expression set point, the creation of multiple orthogonal regulation systems for simultaneous control of multiple genes, and the ability to operate in multiple cell types (FIG. 1C).

Earlier work demonstrated IFFL circuits could generate dosage-invariant expression over a 50- or 100-fold range in bacteria and mammalian cells, respectively. However, these systems required expression of additional proteins, complicating their routine use. miRNA could be an ideal regulator for an IFFL dosage compensation system, as it can be expressed from within an intron, or from compact transcripts. Bleris et al. demonstrated that a miRNA-based IFFL could achieve dosage compensation. Strovas et al. improved on this by incorporating a natural miRNA and multiple repeats of its target sequence within the gene. This reduced expression variation in single-copy integrations, and achieved dosage compensation over a ˜20-fold range, at the cost of potential crosstalk with endogenous genes. Most recently, Yang et al introduced an “equalizer” architecture that combined transcriptional negative feedback through the TetR protein with miRNA. This extended the range of effective dosage compensation, but required expression of the bacterial TetR protein, adding complexity and potential immunogenicity. Despite much work, it has remained unclear what sequence features are sufficient to enable orthogonal, tunable, dosage compensating miRNA IFFLs, and as a result a broadly useful toolkit of such circuits does not yet exist.

A set of miRNA-based dosage compensation systems termed DIMMERs (Dosage Invariant miRNA-Mediated Expression Regulators) was engineered that fulfills this need. These circuits can use specific configurations of the miRNA expression cassette and its target sequences, and take advantage of the ability to achieve multivalent miRNA regulation through the natural TNRC6 scaffold system. They can allow systematic tuning of expression levels by modulating the number of miRNA cassettes, numbers of target binding sites, and miRNA-target site complementarity. Further, they can be used to orthogonally regulate multiple genes in the same cell, and operate similarly across different cell types. A toolkit of ten mutually orthogonal ready-to-use expression systems was engineered that can be incorporated into diverse systems. Finally, their utility was demonstrated for CRISPR imaging and super-resolution protein imaging modalities. DIMMERs provided herein can allow routine research and biotechnology applications to operate with greater precision, control, and predictability.

### Results

### Mathematical Modeling Identifies Parameter Regimes for Dosage Compensation

It was first set out to identify the general conditions under which miRNA-based IFFL circuits can achieve dosage compensated target expression (FIG. 1B, Supplementary Modeling). A minimal model was developed based on several assumptions: First, it was assumed that pri-miRNA and the target mRNA are each transcribed constitutively at a fixed ratio, and in direct proportion to gene dosage. Second, it was assumed that a constant total rate, per gene copy, of RISC complex production. This rate incorporates the combined process of pri-miRNA transcription, post-transcriptional processing, and binding to Argonaute proteins. Third, it was assumed that the RISC and its target mRNA bind reversibly to form a RISC-mRNA complex. Finally, it was assumed that this complex can cause degradation of the associated mRNA.

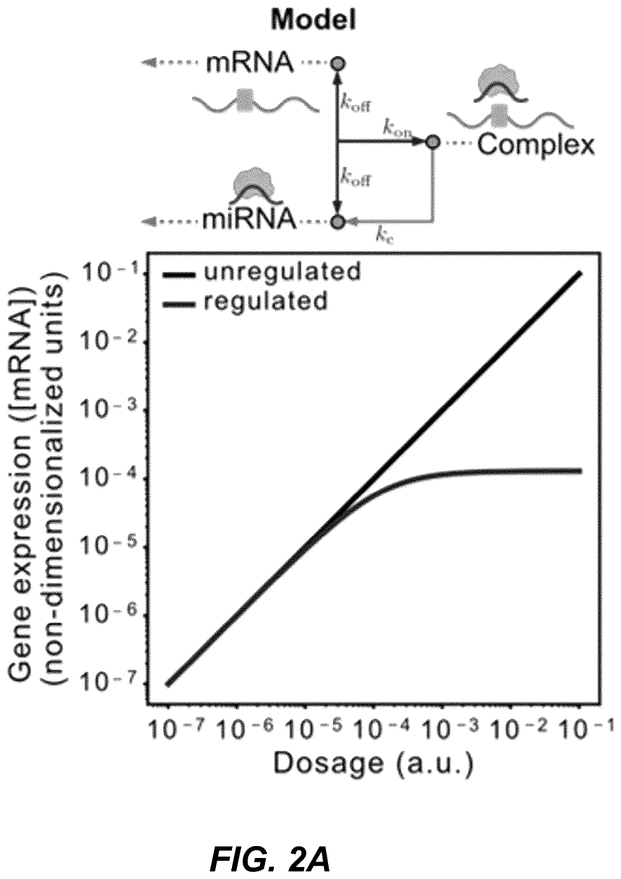

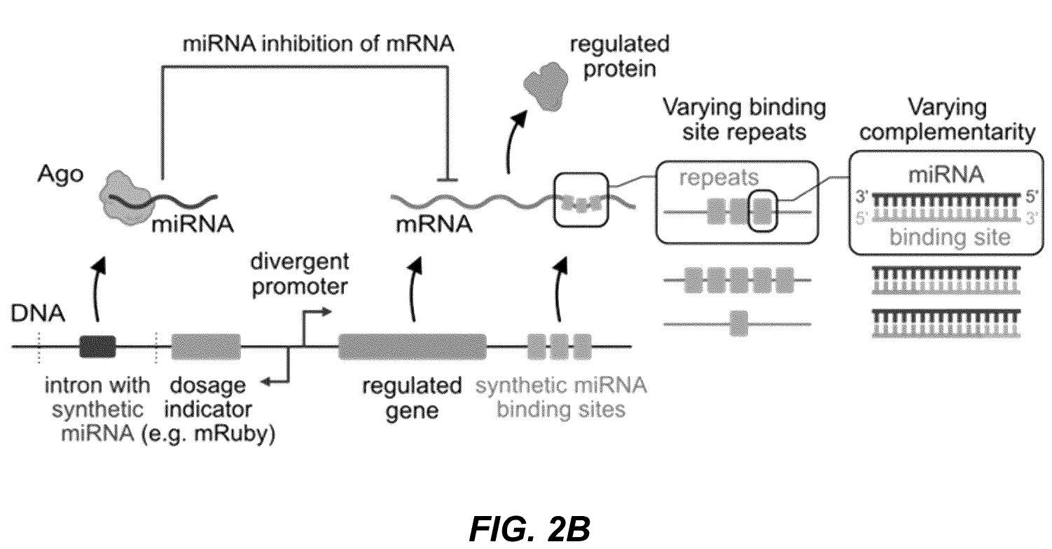

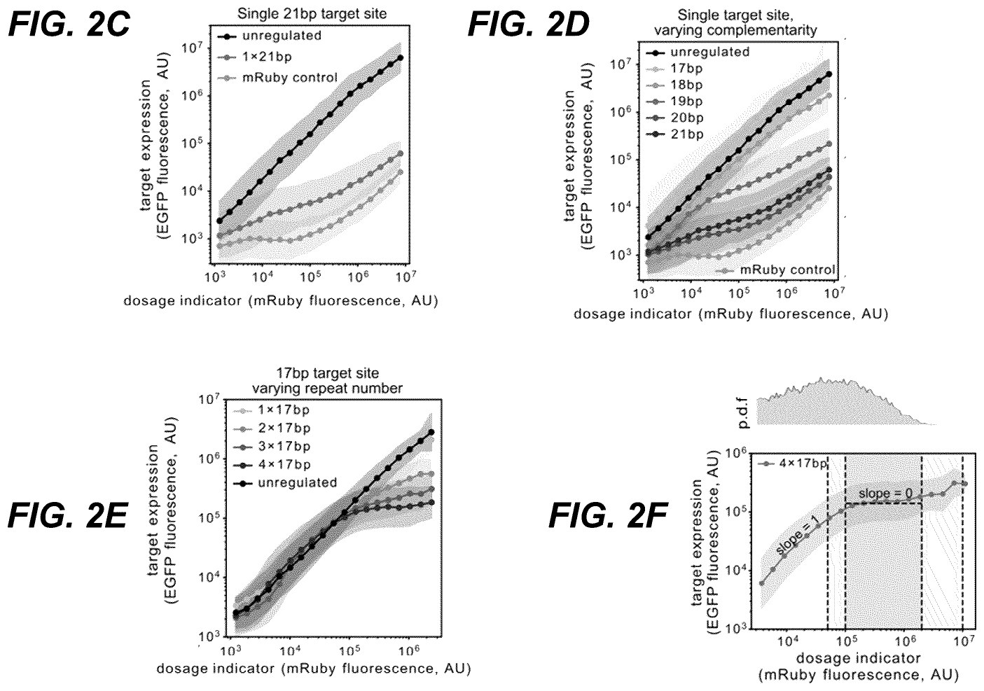

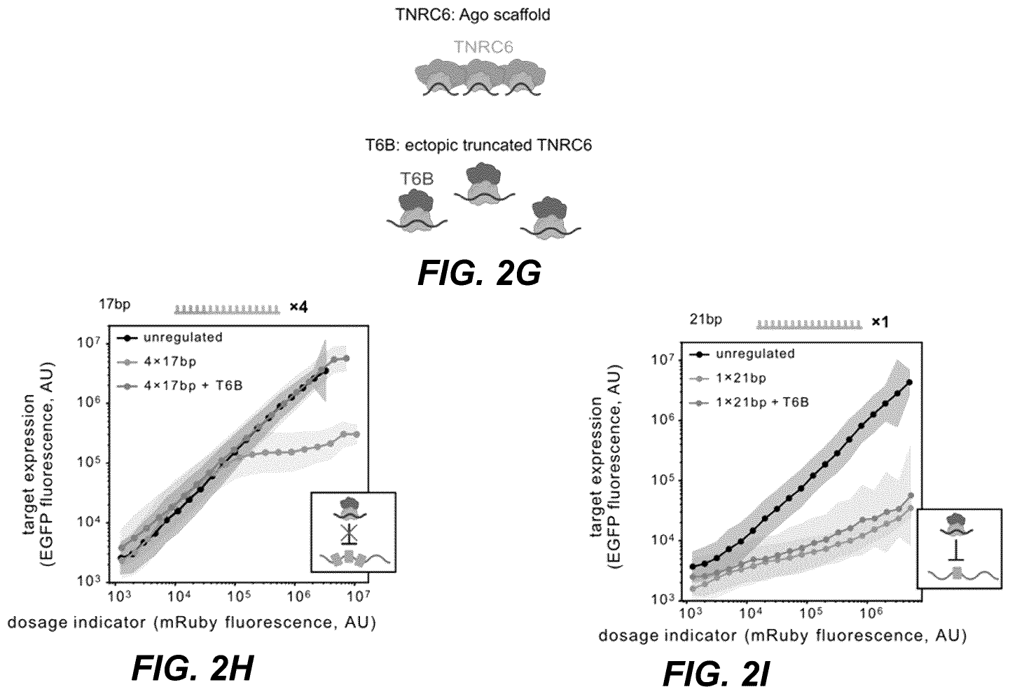

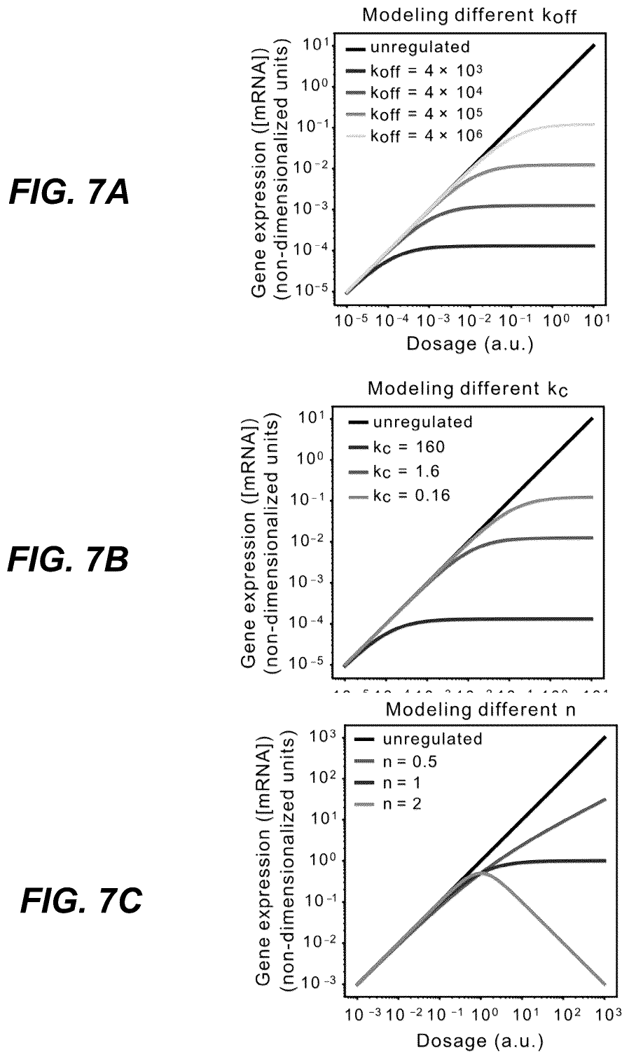

This model exhibited dosage-invariant expression profiles (FIG. 2A). More specifically, target protein expression levels initially increased linearly at low gene dosage but approached a dosage independent limiting expression level, at a value of K/k_{c} (FIG. 2A). Altering the values of the binding or unbinding rates of the mRNA and the miRNA, or the inhibiting strength of the miRNA could be used to tune the steady state level of the mRNA while maintaining compensation (FIGS. 7A-B). It was noted that ultrasensitive or sub-linear regulation of mRNA by RISC can lead to non dosage-invariant profiles (Supplemental Modeling, FIG. 7C). Nevertheless, these results suggest that under general conditions, miRNA-based IFFLs could achieve gene dosage invariant protein expression.

miRNA-Based IFFLs Generate Dosage-Independent Expression

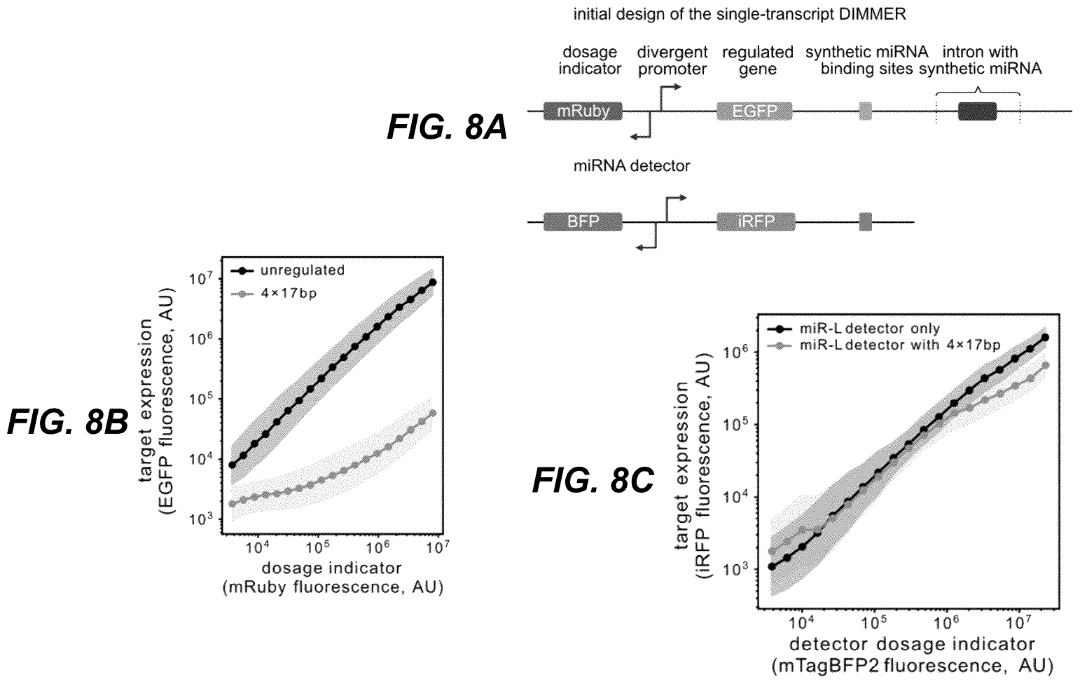

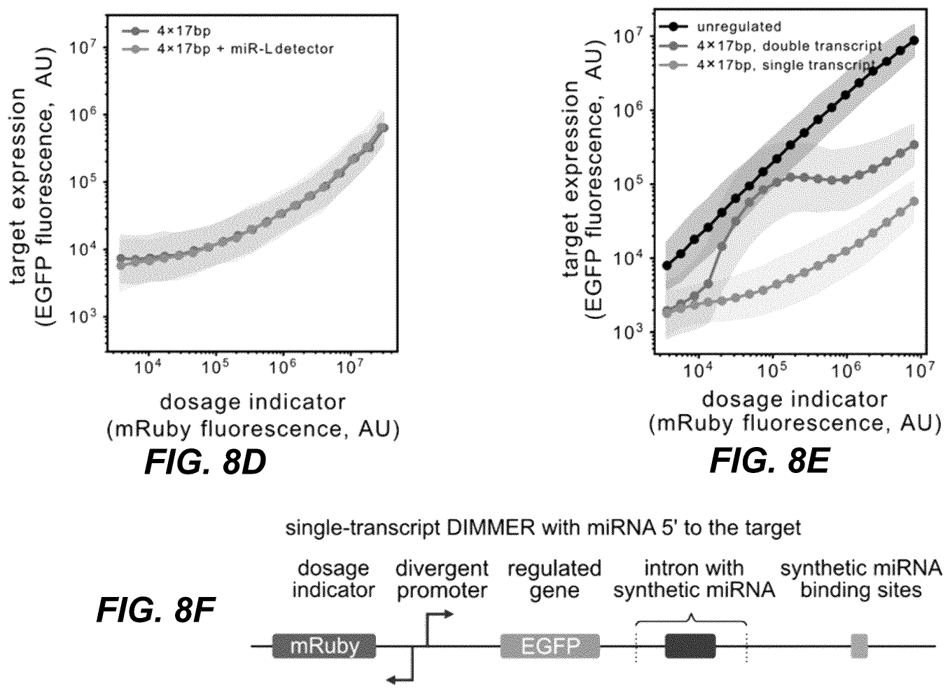

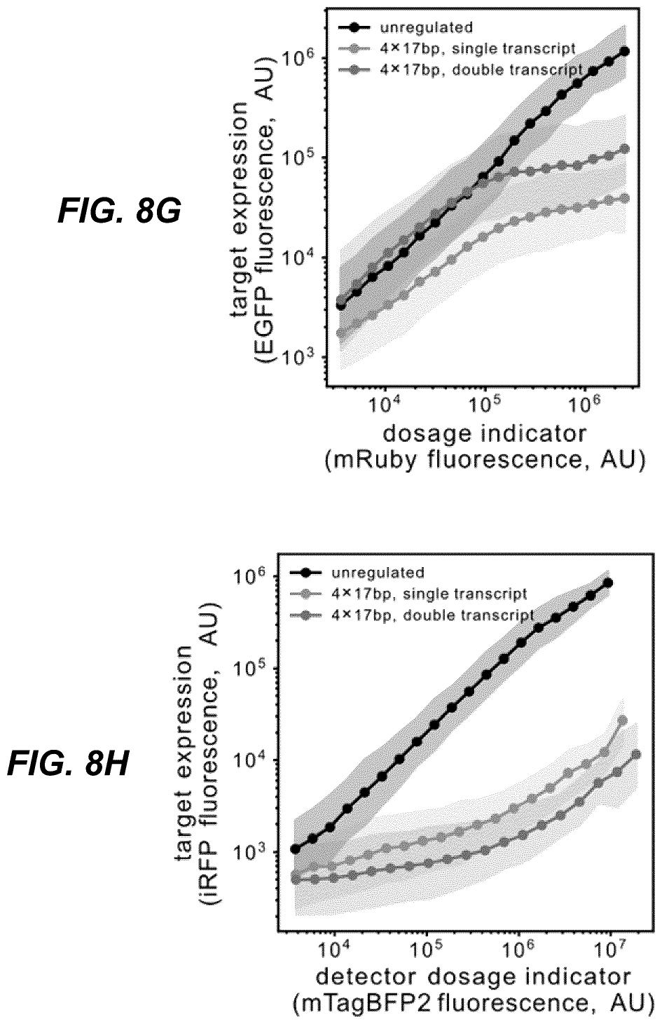

Based on these modeling results, a set of miRNA-based IFFLs was designed and built. After exploring multiple circuit configurations, a design in which the miRNA is expressed on a separate adjacent transcript from the same DNA region (FIG. 2B) was focused on in part because it provides flexibility in the relative miRNA and target gene expression levels (FIG. 8).

To implement the circuit, a platform was constructed comprising a divergent promoter pair (FIG. 2B). One promoter (oriented left in FIG. 2B) constitutively expressed a fluorescent protein dosage indicator, mRuby3, along with a synthetic miRNA. To create the miRNA expression cassette, the miR-E backbone for pri-miRNA expression was inserted into a synthetic intron. The second promoter (oriented right in FIG. 2B) constitutively expressed the target gene, EGFP. To allow regulation by the synthetic miRNA, one or more target miRNA sites of varying complementarity was incorporated in the 3′UTR of the target gene. This compact two-transcript construct allowed systematic analysis of different miRNAs and target site configurations.

To implement miRNA regulation orthogonal to natural miRNAs, a previously described miRNA sequence targeting Renilla luciferase (miR-L), together with a single copy of its fully complementary 21 bp target site, was first used. U2OS cells were transiently transfected with the resulting construct, analyzed cells by flow cytometry after 48 h, and plotted target EGFP expression versus gene dosage, as indicated by mRuby3 (FIG. 2C). Compared to an unregulated control with no miRNA target site, the IFFL strongly reduced target EGFP expression, as expected (FIG. 2C). However, the circuit failed to achieve the dosage compensation behavior anticipated from mathematical modeling (FIG. 2A).

It was next asked whether the lack of dosage compensation could relate to the strongly repressing regime produced by full miRNA-target complementarity. A set of IFFL variants was designed which progressively reduced complementarity of the single target site from 21 to 17 bp (Table 1). Designs with reduced 3′ complementarity showed weak or no repression of target expression, particularly below 19 bp (FIG. 2D), while those that did efficiently repress retained strong repression comparable to that of the full length 21 bp construct. Nevertheless, within this set, no construct achieved full dosage invariance. Thus, modulation of complementarity alone was not sufficient to provide dosage compensation.

The loss of regulation at reduced complementarity contrasted with the well known regulatory capacity of miRNAs with much shorter complementary regions of only ˜8 bp. One mechanism to enable specific regulation with short sequences involves multivalent recognition of multiple target binding sites on the same mRNA. TNRC6 is a scaffold protein that enables multivalent recognition by simultaneously binding to multiple miRNA-loaded Argonaute (Ago) complexes (FIG. 2G). Consistent with a role for multivalent regulation, tandem repeats of two to four copies of the 17 bp target site progressively increased regulation, and strongly reduced dosage sensitivity at higher expression levels (FIG. 2E). For example, with 4 tandem binding sites, target expression increased by only 4-fold over a 200-fold range of dosages (FIG. 2F). A “tail” of elevated expression at the highest dosages may reflect saturation of miRNA-associated machinery, as observed in other studies. Together, these results show that a miRNA-based IFFL based on 17 bp of miRNA-target complementarity and 4 tandem binding sites can achieve nearly complete compensation over more than two orders of magnitude of dosage variation.

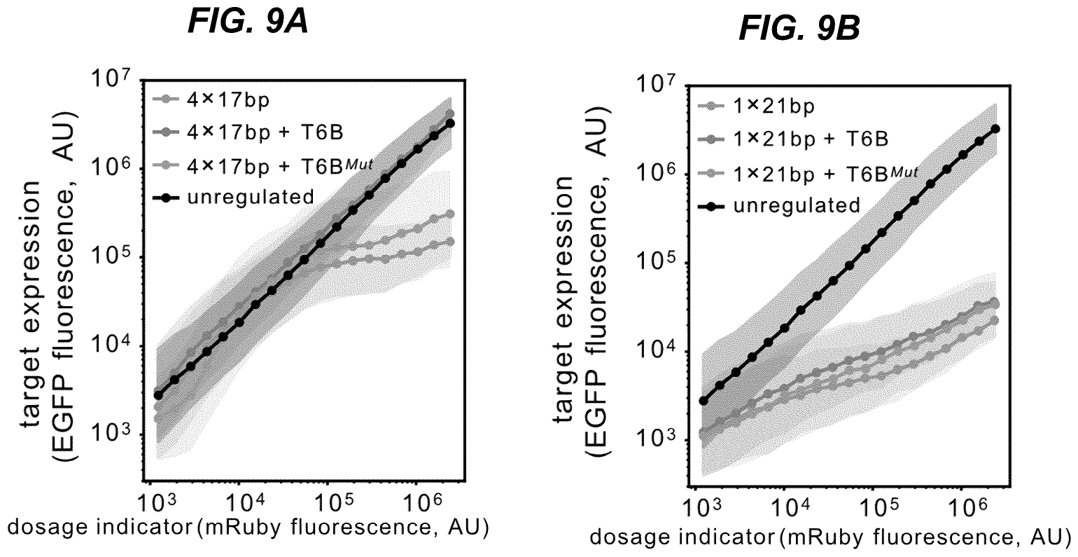

To find out whether this compensation behavior was dependent on TNRC6, a previously identified fragment of the natural TNRC6B protein, the T6B peptide that competitively inhibits TNRC6 activity (FIG. 2G), was taken advantage of. When co-transfected with the 4×17 bp IFFL, the T6B inhibitory peptide abolished regulation, producing dosage-dependent expression nearly identical to that produced by an unregulated construct (FIG. 2H). By contrast, the T6B inhibitory peptide had little effect on the single fully complementary 21 bp construct, suggesting that it regulates in a TNRC6-independent manner (FIG. 2I). Finally, negative control mutant variants of T6B lacking the Ago2-binding domain failed to abolish regulation, as expected (FIG. 9A). Together, these results suggest that the 4×17 bp and 1×21 bp designs respectively operate through TNRC6-dependent and TNRC6-independent mechanisms. Because of their ability to limit expression, these circuits are termed herein DIMMERs. More generally, these results indicate that multivalent regulation through multiple, individually weak, miRNA binding sites can achieve strong regulation and dosage compensation within the context of the IFFL circuit.

### DIMMER Circuits Allow Tuning of Dosage-Independent Expression Levels

An ideal control system would allow not only dosage invariance but also control of saturating expression level. In principle, this could be achieved by modulating the complementarity of repeated target sites, the number of miRNA cassettes within the synthetic intron, or the strength of the promoter controlling expression of the miRNA cassette. Each of these parameters was systematically varied and its effects on expression was analyzed.

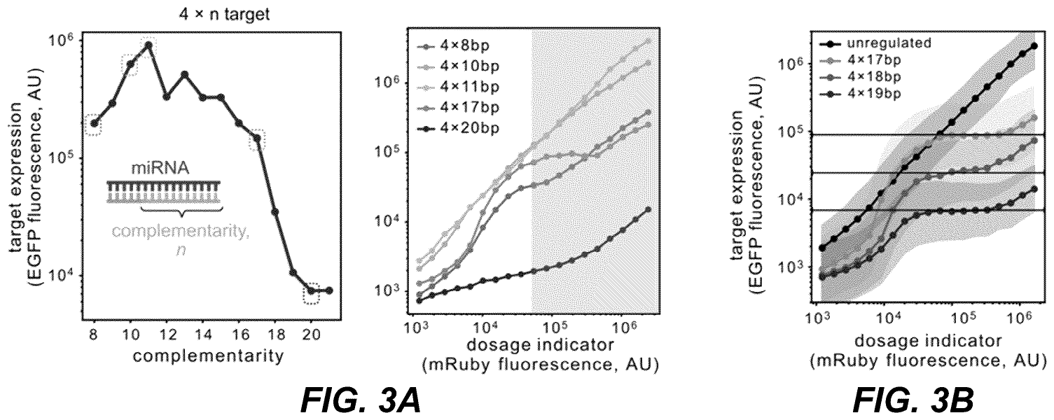

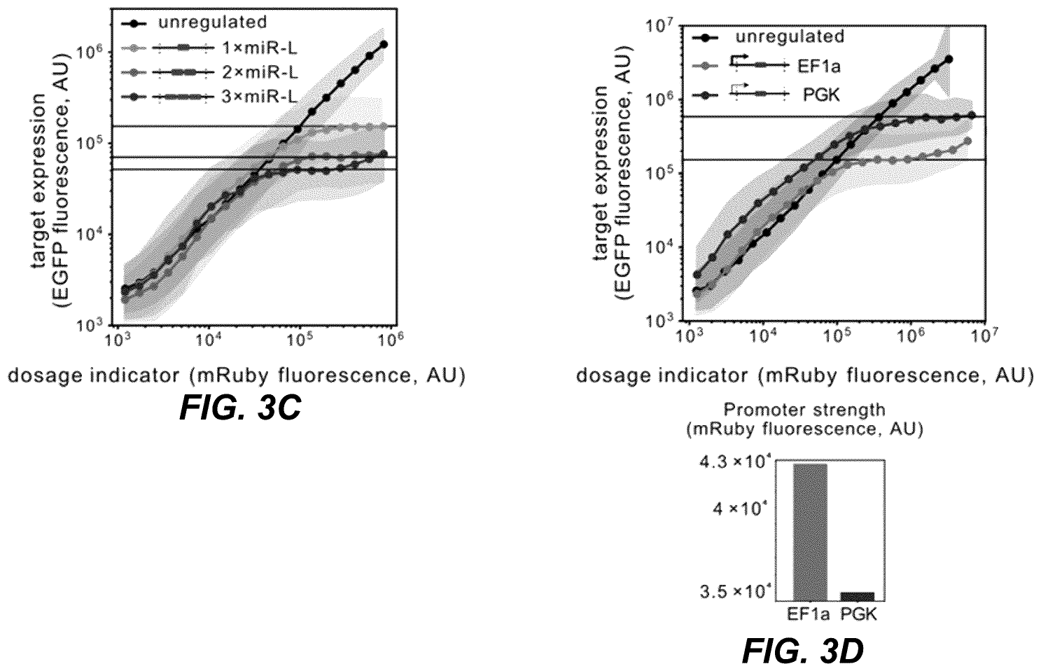

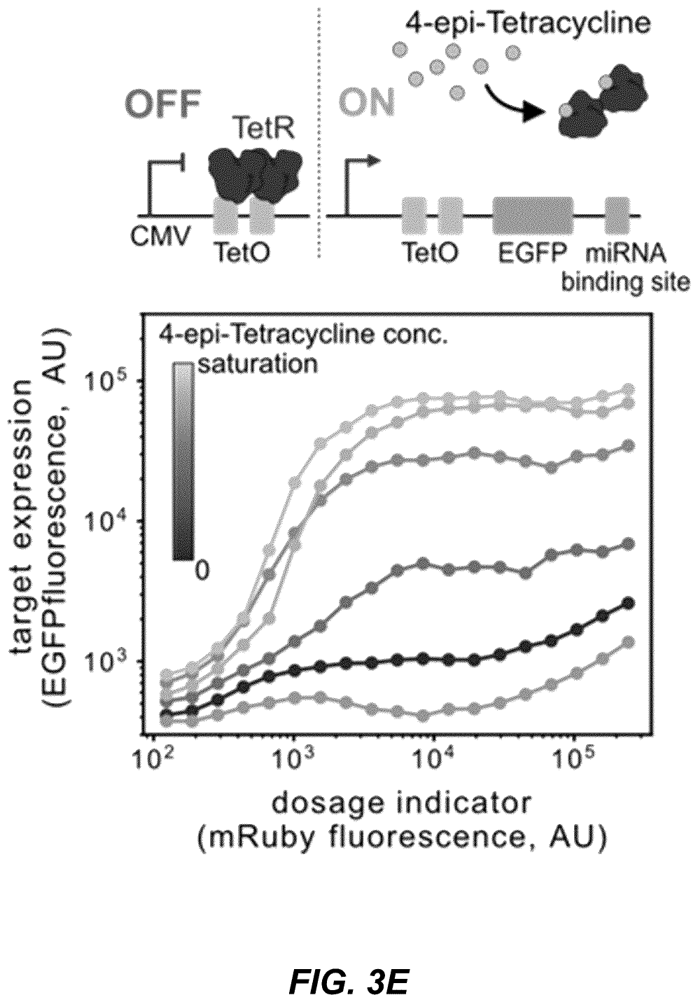

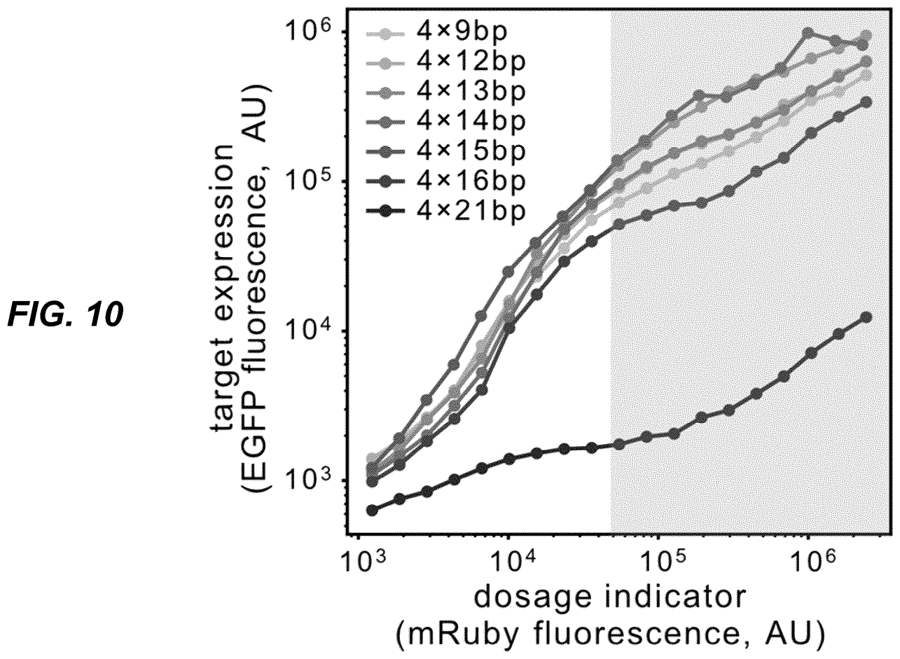

First, the complementarity of the miR-L target sites was varied from 8 to 21 bp, in each case incorporating 4 tandem target site copies (FIG. 3A, FIG. 10). At each length, the expression of the IFFL-regulated target was quantified (FIG. 3A, left panel) at high gene dosage (FIG. 3A, right panel, shaded region). Repression was modest at 8 bp, diminished with increased complementarity in the central region, and then strengthened again as more complementarity was added after the central region (FIG. 3A, left panel). These results are broadly consistent with previous observations showing that miRNA inhibition does not increase monotonically with complementarity. For the miR-L target site, repression was most sensitive at 16-20 base pairs of complementarity (FIG. 3A). In fact, three designs—4×17, 4×18, and 4×19—achieved evenly spaced, dosage invariant expressions spanning more than an order of magnitude in saturating expression level (FIG. 3B).

Next the number of copies of the miRNA expression cassettes in the synthetic intron was varied, effectively modulating the stoichiometric ratio of miRNA to mRNA (A second design feature of these circuits is the separation of miRNA and target 3C). Compared to a single copy, two or three copies of the miRNA reduced expression by 2-fold and 3-fold, respectively, while preserving dosage compensation, providing a means of fine-tuning expression control.

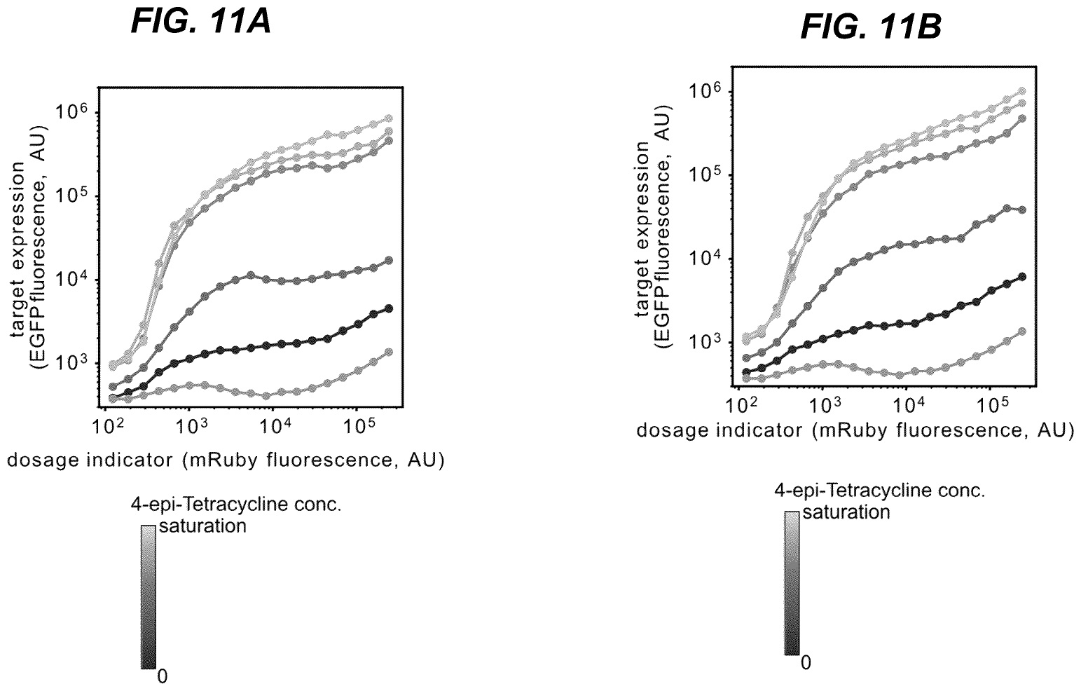

Finally, the promoter controlling expression of the miRNA cassette was varied. Compared to EF1α, the weaker PGK promoter allowed ˜3.5-fold more target gene expression at a given dosage level (FIG. 3D). Nevertheless, dosage compensation was preserved here too. Further, it was questioned whether our circuit works in an inducible system setting. The 4-epi-tetracycline tuned promoter was used to express the target and the circuit's performance in TRex cells was tested (FIG. 3E). By modulating the concentration of 4-epi-tetracycline, the setpoint of the circuit was able to be varied by ˜2 orders of magnitude while preserving dosage compensation (FIG. 3E, FIG. 11). Taken together, these results demonstrate that four distinct mechanisms can be used to tune expression level in a coarse or fine manner.

### Orthogonal Dosage Compensation Circuits Allow Independent Control of Target Genes

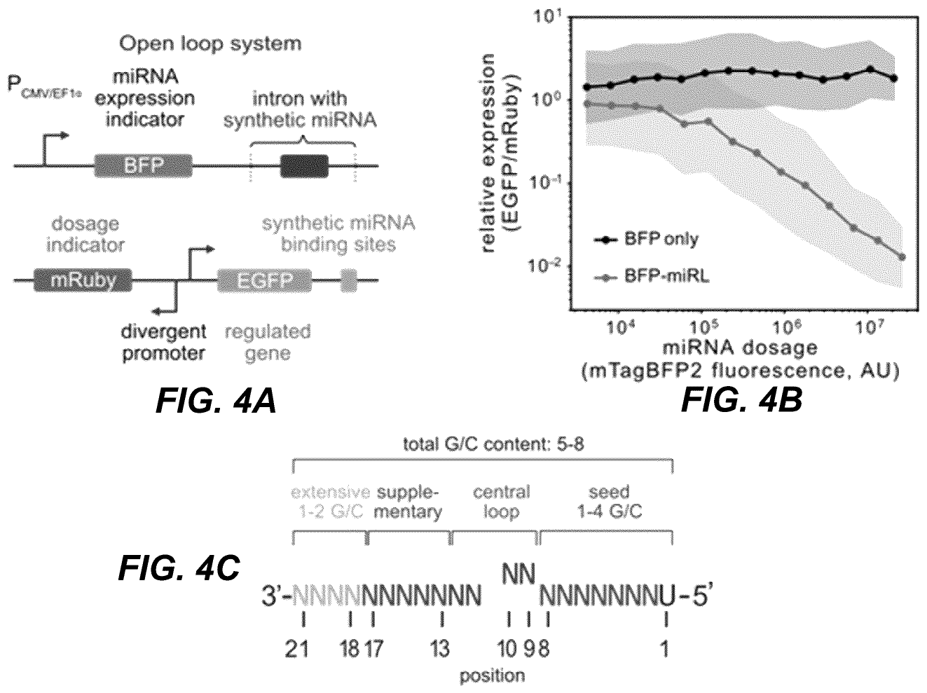

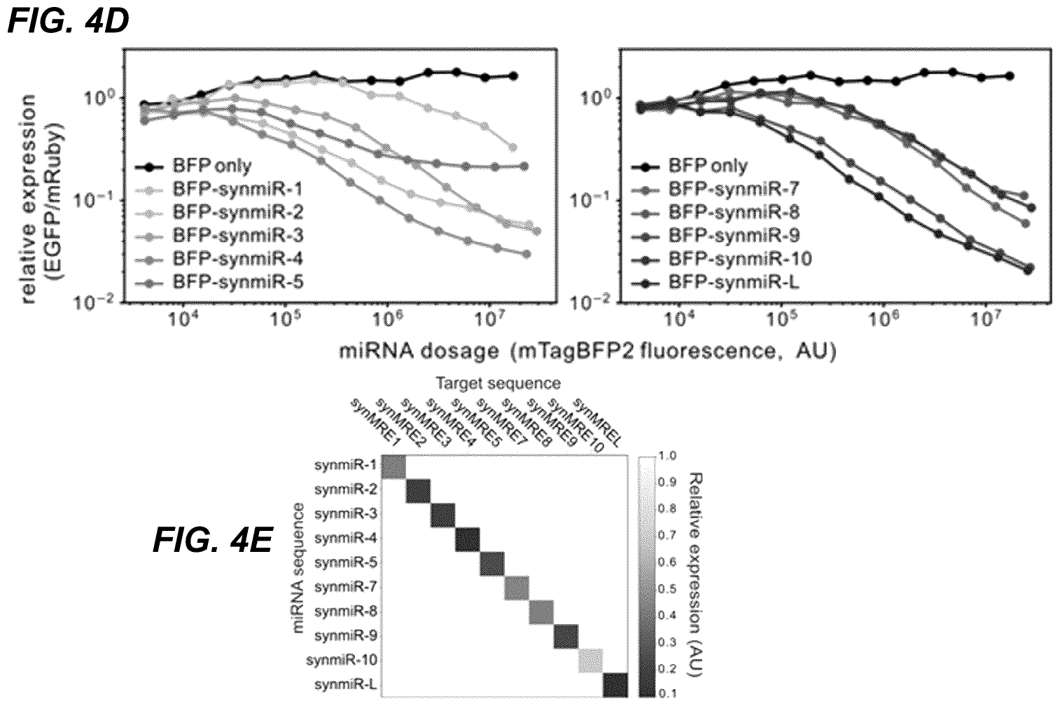

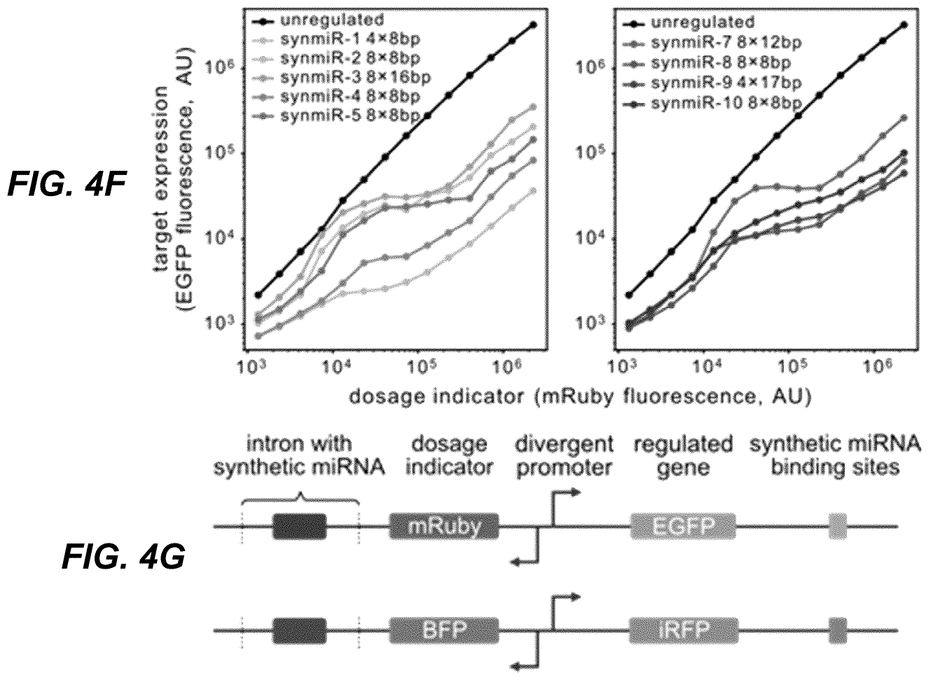

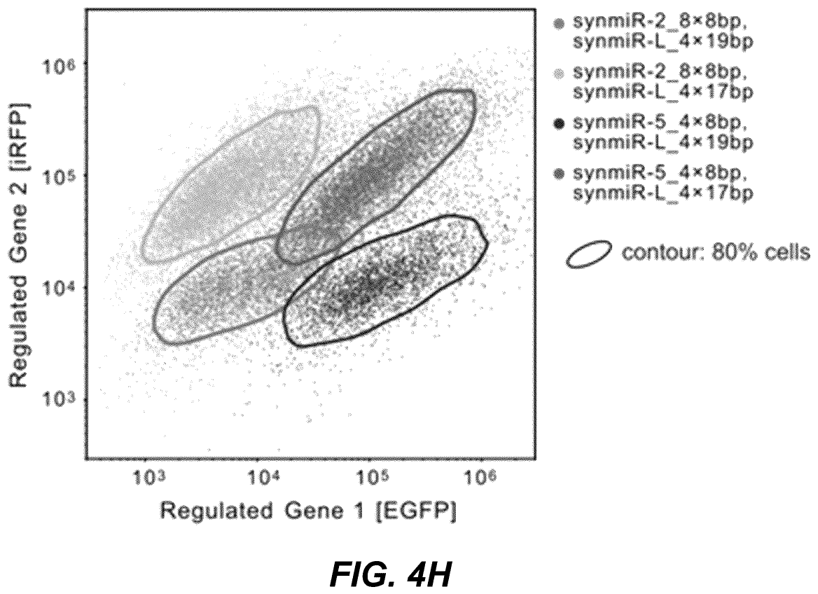

Engineered genetic systems increasingly require multiple genes and transcripts, many or all of which may benefit from dosage-independent control. It was therefore set out to construct an expanded set of orthogonal synthetic miRNA-target site pairs, which is termed synmiRs. To design synmiRs, started by generating five random miRNA sequences, each with an initial A in the miRNA, a cognate U at the 3′ end of a single target site and 25% GC content, similar to the structure of miR-L. The dose-response curve was then measured for each candidate miRNA. To do so, an “open loop” system was designed, which allows independent control of miRNA expression and measurement of its effect on a target miRNA reporter gene (FIG. 4A). For a single fully complementary miR-L site, it was seen that the inhibition increases with the dosage of the miRNA (FIG. 4B), consistent with the earlier closed loop results (FIG. 2C). Of the five sequences initially considered, synmiRs 1, 4, and 5 repressed by at least an order of magnitude relative to a control lacking the miRNA (Table 1, FIG. 4D). By contrast, synmiRs 2 and 3 achieved less substantial repression (A second design feature of these circuits is the separation of miRNA and target 12A). Inspection of these designs revealed subsequences with two or more A/T pairs in the extensive region. It was hypothesized that these A/T dinucleotides could potentially trigger the miRNA degradation machinery to recognize the miRNA tailing and trimming signal, resulting in miRNA destabilization. Consistent with this hypothesis, an A to G substitution at position 20 in synmiR-2 or at position 19 in synmiR-3 restored miRNA inhibition of target gene expression (FIG. 4D).

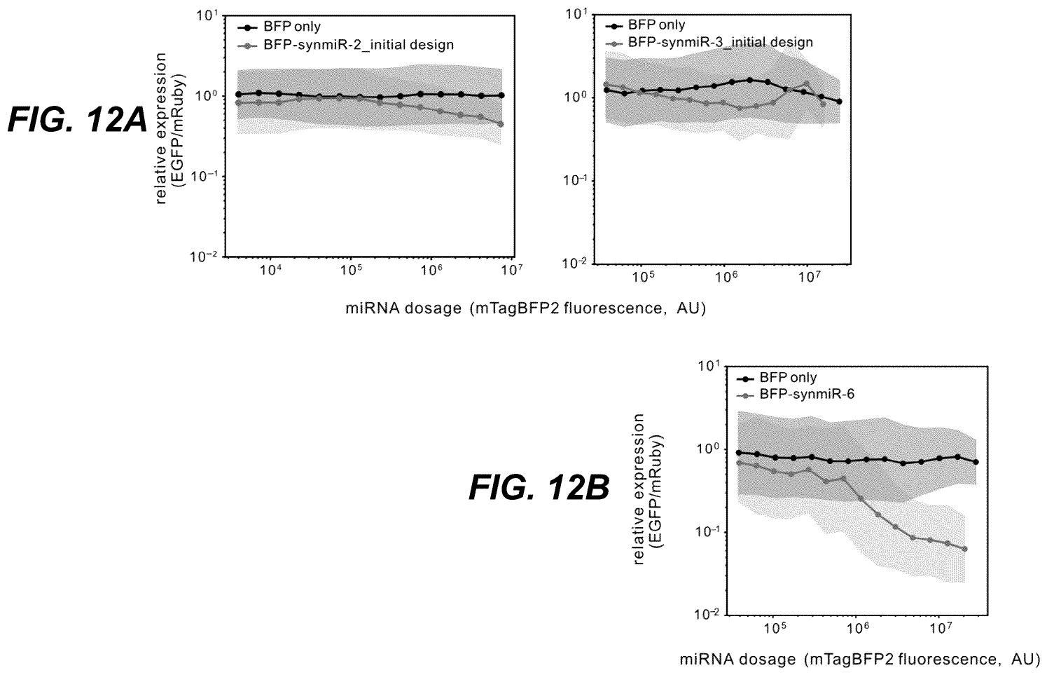

Based on these results, an empirical two-step sequence design algorithm was formulated (FIG. 4C). In the first step, random 21 bp candidate miRNA sequences constrained to have a 5′U in the mature miRNA were generated. In the second step, based on known requirements for miRNA loading, sequences were constrain to a total 5-8 GC nucleotides, 1-4 in the seed region, and 1-2 in the extensive region (FIG. 4C). Five candidate sequences (synmiRs 6-10) were generated and tested based on this simple algorithm and analyzed their open loop behavior (FIG. 4D, FIG. 12B). When used as a single fully complementary site, all five sequences generated strong repression, comparable to that of miR-L. However, one sequence, synmiR-6, was discarded due to its similarity to the human endogenous miRNA hsa-mir-5697 (Methods). Altogether, these results produced a set of ten miRNA sequences that were capable of strong repression in their fully complementary form (FIG. 4D). Pairing each of these ten miRNAs with all ten of the target sequences in the open loop system revealed strong orthogonality in regulation, as desired (FIG. 4E).

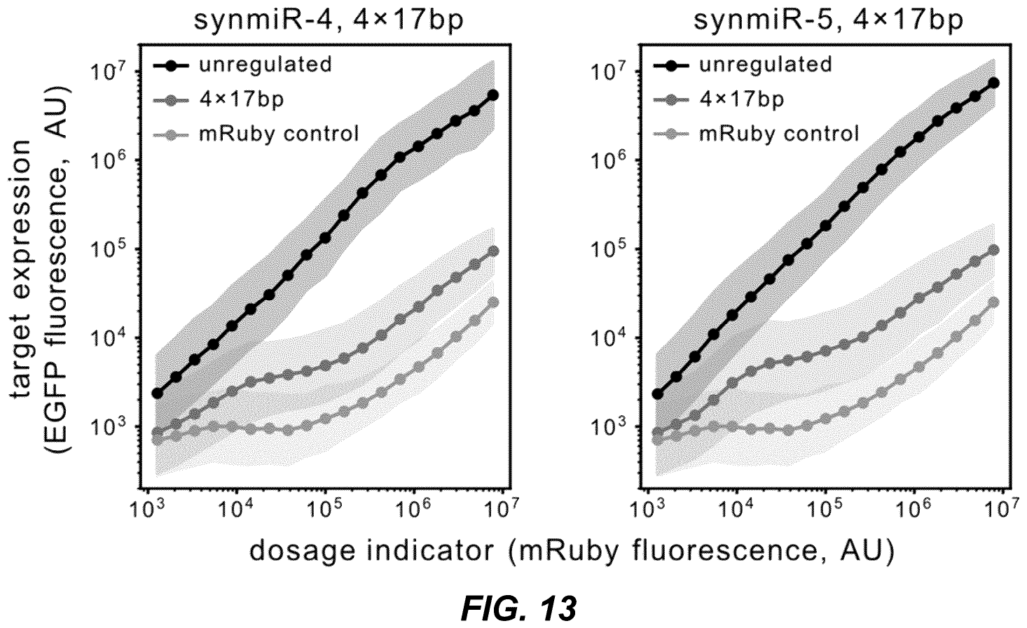

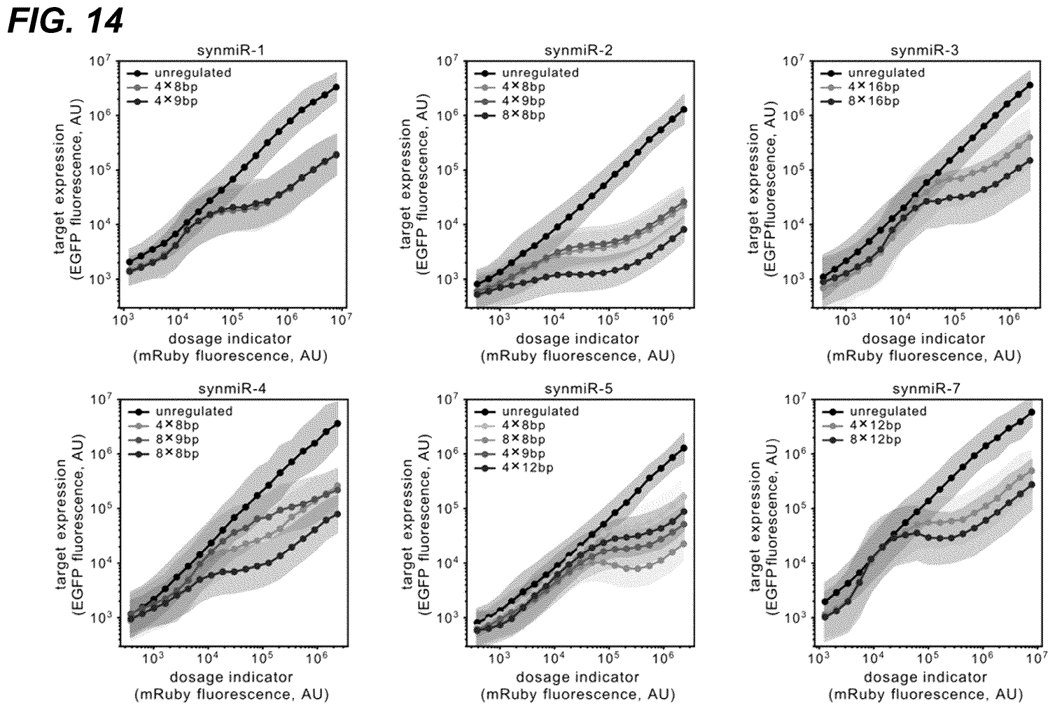

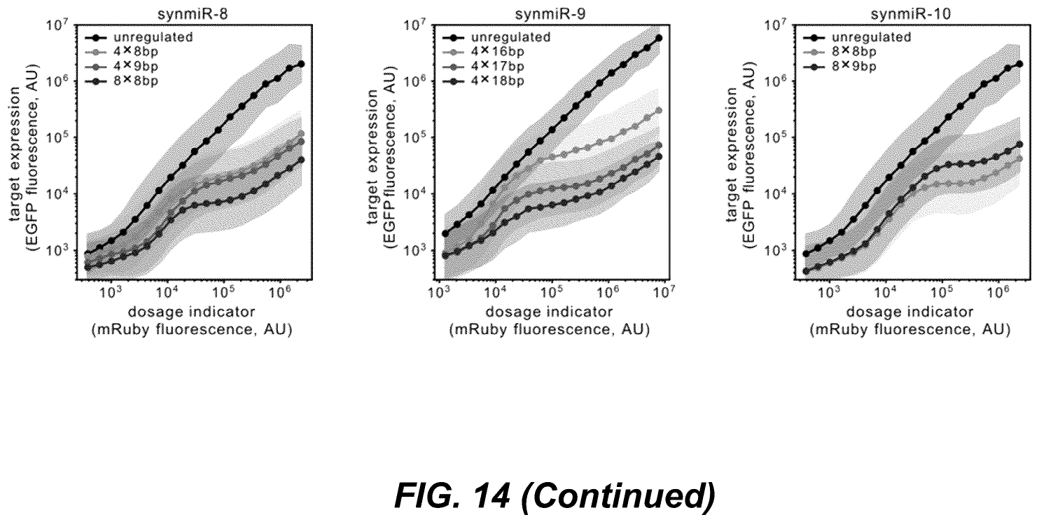

Using these synmiR sequences, a set of ten orthogonal dosage compensation systems was developed, using the framework in FIG. 2B. Based on our earlier analysis of miR-L (FIGS. 2-3), the complementarity and the number of tandem target sites was varied for each sequence design. Different sequences required alternate configurations to produce dosage compensation. For example, with synmiR-4 and synmiR-5, the 4×17 bp configuration produced a strong inhibition profile akining more with the fully complementary target (FIG. 13). It was reasoned that this could reflect higher GC content in the seed and supplementary regions of these two miRNAs compared to miR-L. This increase in GC content allowed shorter 8 or 9 bp target sites, present in 4-8 repeats, to successfully produce dosage compensating designs for these miRNAs (FIG. 4F). A similar process was repeated for the other sequences and eventually identified nine additional closed-loop systems that exhibit substantial levels of dosage compensation (FIG. 4F). In general, regulatory behavior was sensitive to both the number and complementarity of target sites (FIG. 14). Critically, however, each of these nine synmiR sequences, as well as the original sequence, was able to generate at least partial dosage compensation in one or more configurations (FIG. 4F), and many could be tuned through these features to different expression setpoints (FIG. 14). These results provide a toolkit of dosage compensating systems and more generally suggest that it should be possible to engineer additional systems with varying expression setpoints.

By using multiple dosage compensation systems together, one should be able to simultaneously and independently specify the expression of multiple target genes (FIG. 1C, panel 2). To test this possibility, a second set of dosage compensation expression systems was constructed using distinct fluorescent reporters (FIG. 4G). Cells were transfected with pairs of systems that had different regulatory setpoints, and the resulting expression profiles of the two regulated target genes was analyzed (FIG. 4H). Four pairs of systems were analyzed. Each produced a distinct two-dimensional expression distribution based on the set-points for the two reporters. This demonstrates that the engineered dosage compensation systems make it possible to specify two-dimensional expression distributions, and suggests that control of higher dimensional distributions of more genes should also be accessible.

### Dosage Compensation Systems are Portable and Minimally Perturbative

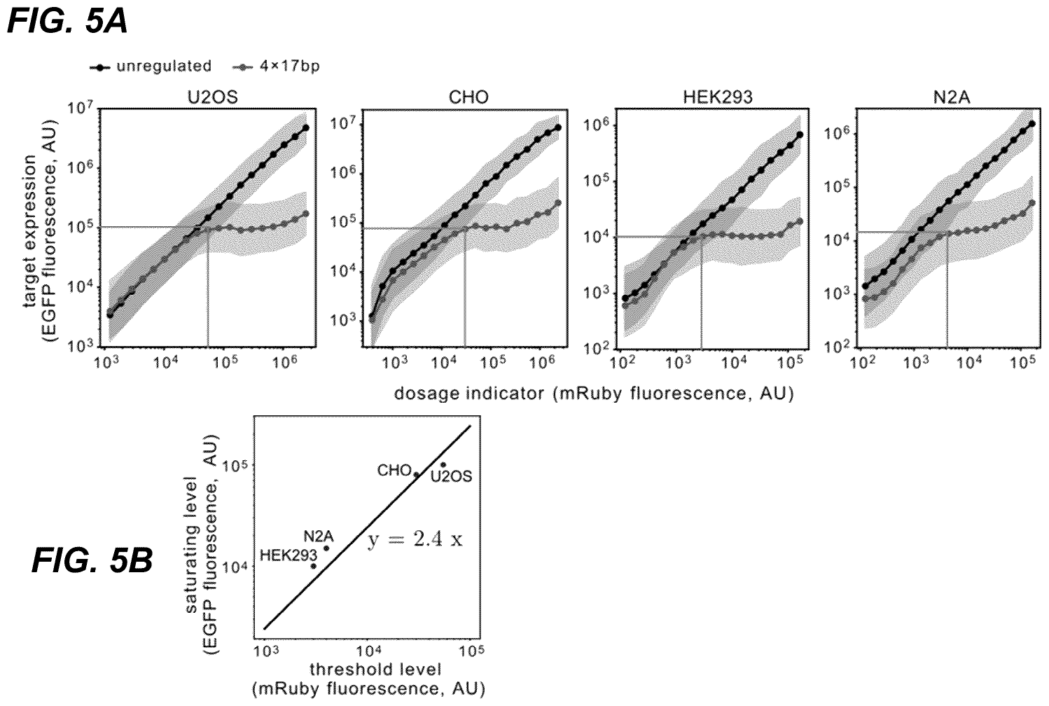

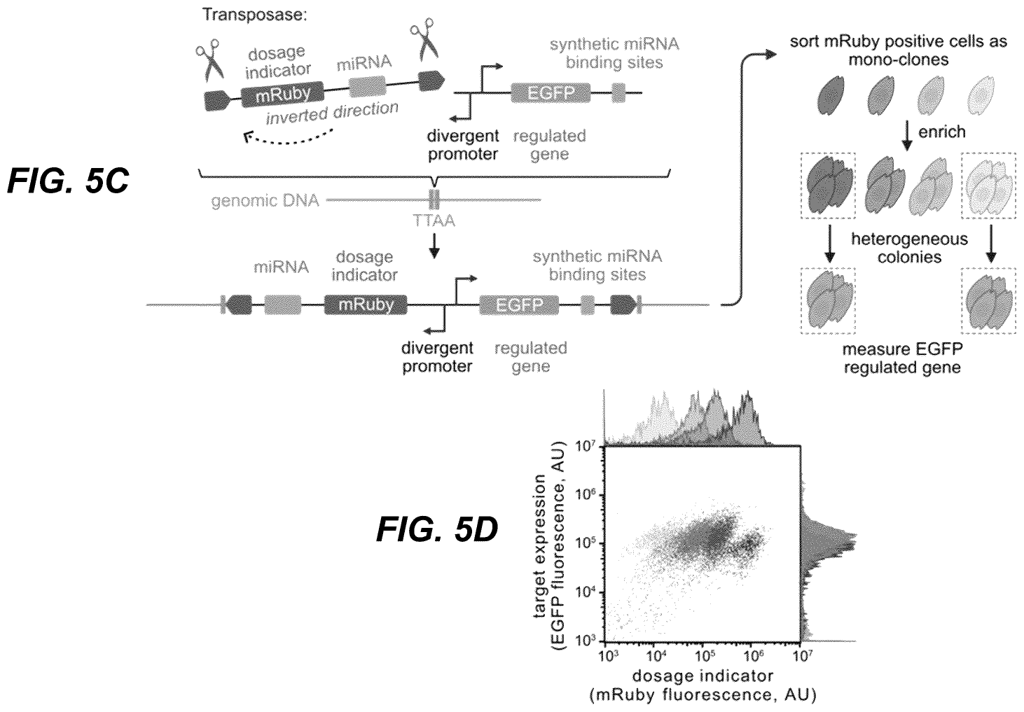

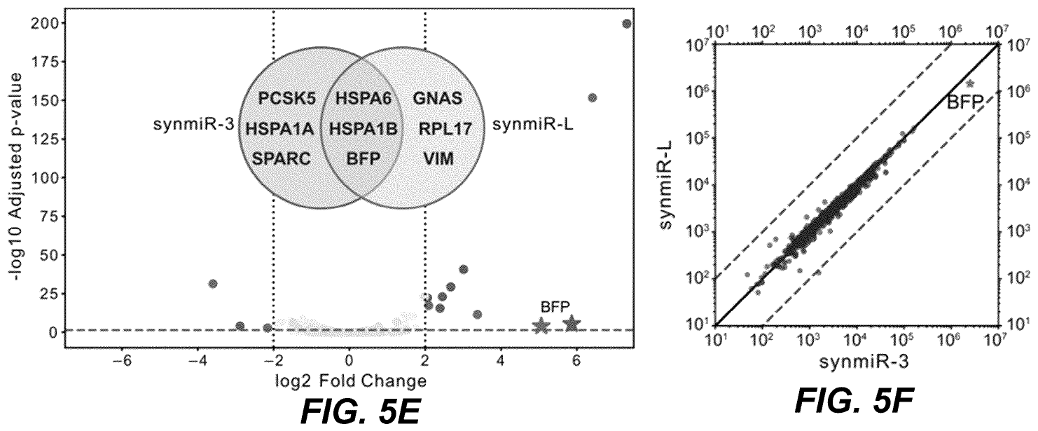

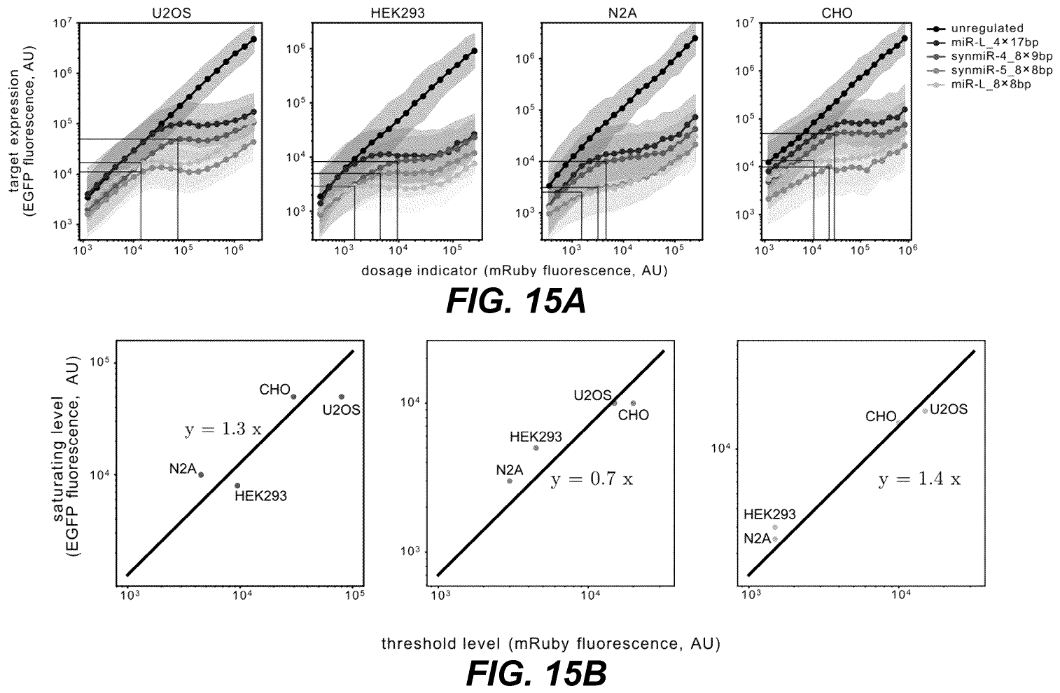

An ideal dosage compensation system would be portable, able to operate similarly across different cell types, function in both transient transfection and genomic integration, and minimally perturb the host cell. To examine these features, several circuit variants were transiently transfected, including the 4×17 miR-L system (FIG. 2E), in four mammalian cell lines: U2OS, CHO-K1, HEK293, and N2A. In each cell line, strong and qualitatively similar dosage compensation was observed (FIG. 5A). Cell lines varied in the threshold dosage at which expression saturated (FIG. 5A, gray vertical line), and in the saturating expression level (FIG. 5A, gray horizontal line), as measured in arbitrary fluorescence units. However, the ratio of these values was conserved (FIG. 5B). Similar results were obtained for other circuits as well, including synmiR-4, with 8 repeats of a 9 bp target site, as well as both synmiR-L and synmiR-5, each with 8 repeats of an 8 bp target site (FIG. 15A). Again, the ratio of the saturating expression level to the threshold dosage was similar, for each construct, across cell lines (FIG. 15B). This suggests a model in which the miRNA circuit functions equivalently in different cell types, but protein expression strengths vary, possibly due to differences in translational capacity or basal protein degradation rates. Together, these results indicate that the dosage compensation circuits can function across different cell contexts.

Stable cell lines are important in research as well as applications like cell therapy. To find out whether dosage compensation circuits could also function in a stable integration context, PiggyBac transposition was used together with the iON system that allows expression only from constructs that have successfully integrated in the genome and undergone site-specific recombination. Mono-clones were then selected, and reporter expression was analyzed by flow cytometry (FIG. 5C). Integration copy numbers varied among clones by over two orders of magnitude, as indicated by mRuby3 fluorescence intensity (FIG. 5D, x-axis). Nevertheless, the cargo EGFP expression remained nearly constant (FIG. 5D, y-axis). Thus, dosage compensation circuits function in stable integration settings.

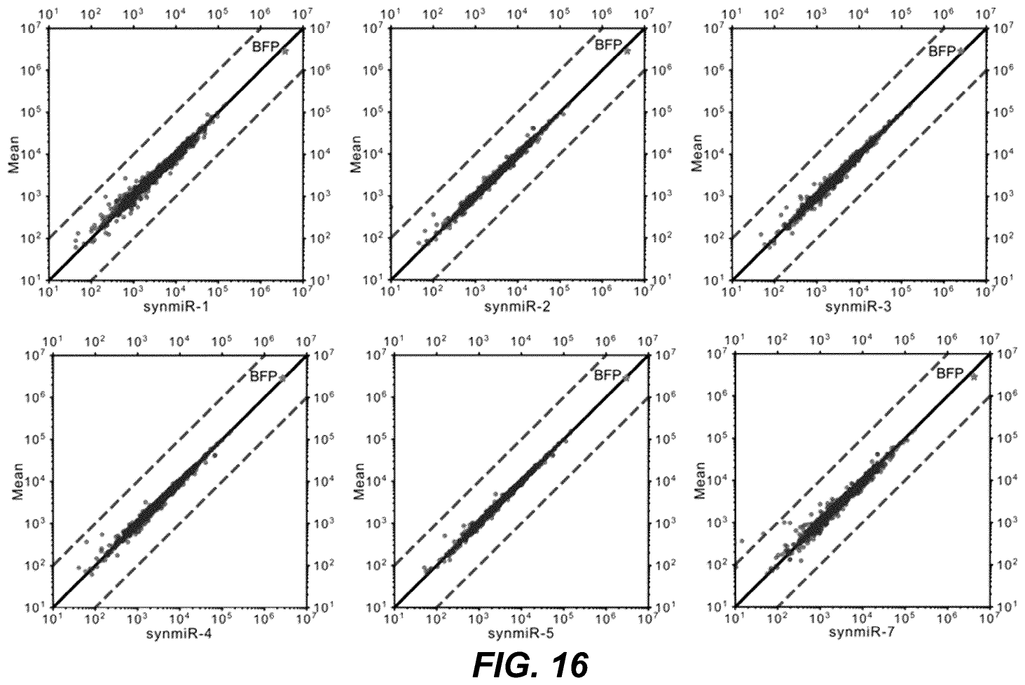

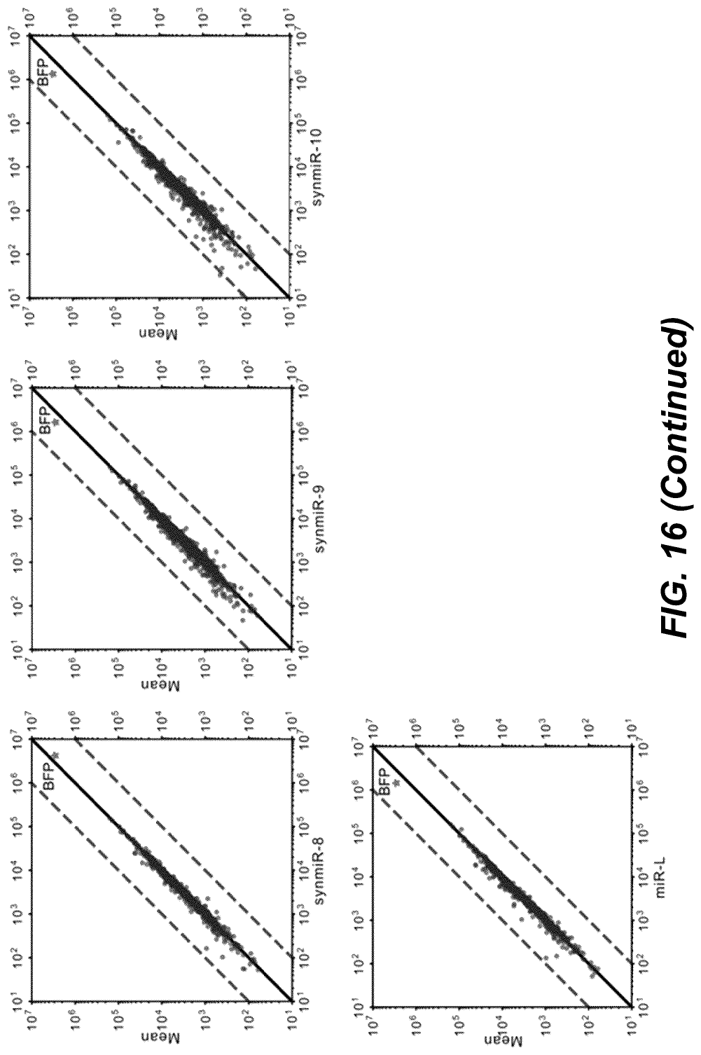

The expression of synthetic miRNAs could in principle perturb endogenous gene expression. To identify such effects, bulk RNAseq was performed on cells transfected with miR-L and each of the 9 orthogonal synmiRs, and compared them to a negative control transfection of a BFP expression vector. Only a few genes were significantly up- or down-regulated by the miRNA (FIG. 5E). These were enriched for heat shock proteins such as HSPA6. Critically, the gene sets up-regulated by different miRNAs exhibited strong overlap (FIG. 5F, Table 8, FIG. 16). Thus, for the synmiRs described here, off-target regulation appears to only reflect non-specific effects of miRNA expression, rather than sequence-specific perturbations.

TABLE 8
Gene Annotation for the significantly differentially
expressed genes suggested by bulk RNAseq

| miRNA | Differentially expressed genes |
|---|---|
| synmiR-1 | HSPA6, PCSK5, HSPA1A, HSPA1B, KRT17, LOLX4, BAG3, SSC4D, |
| | FADS2, GNB2, TUBA1A, TNNC1, CLPTM1L, FOXD1, CCN2 |
| synmiR-2 | HSPA6, PCSK5, HSPA1A, HSPA1B, RPL17 |
| synmiR-3 | HSPA6, PCSK5, HSPA1A, HSPA1B, SPARC |
| synmiR-4 | HSPA6, PCSK5, HSPA1A, HSPA1B, CLPTM1L, POLR2L |
| synmiR-5 | HSPA6, PCSK5, HSPA1A, HSPA1B, CLPTM1L, SSC4D, RPL17, TNNC1 |
| synmiR-7 | HSPA6, PCSK5, HSPA1A, HSPA1B, BAG3, GNB2, AP2S1, CLPTM1L, |
| | RHOC, HDLBP |
| synmiR-8 | HSPA6, PCSK5, HSPA1A, HSPA1B, RPL17, LOXL4, FADS2, KRT17, |
| | RPS2, SSC4D, SPARC, DDX5, TNNC1 |
| synmiR-9 | HSPA6, LOXL4, FADS2, KRT17, PCSK5, RPL17, HSPA1B, SSC4D, |
| | DKK3, LOXL1, TPM1, TUBA1A, BCAM, SPARC, MAGED1, SEMA6B, |
| | TNNC1, AP2S1, RPS2, RPS19 |
| synmiR-10 | HSPA6, LOXL4, FADS2, KRT17, PCSK5, GNAS, RPL17, SSC4D, DKK3, |
| | BCAM, MAGED1, DDX5, SPARC, TUBA1A, YWHAZ, TNNC1, AP2S1, |
| | ACTG1, MRFAP1, RPS2, VIM |
| miR-L | HSPA6, GNAS, HSPA1B, RPL17, VIM |

Shared differentially expressed genes are in bold.

### Dosage Compensation Systems can Suppress Background to Enhance Biological Imaging

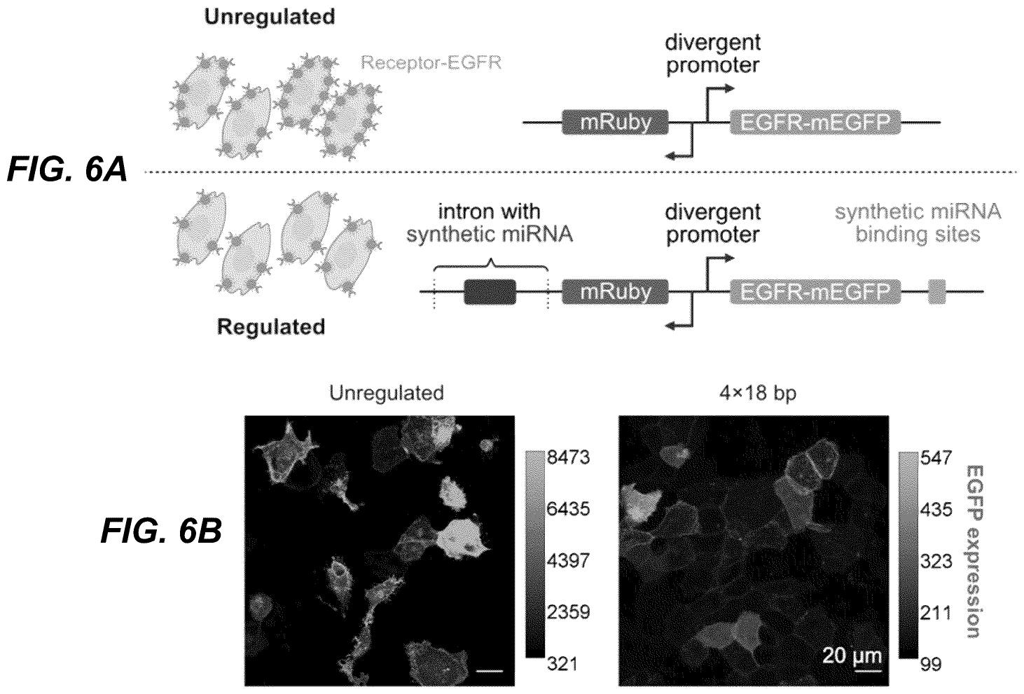

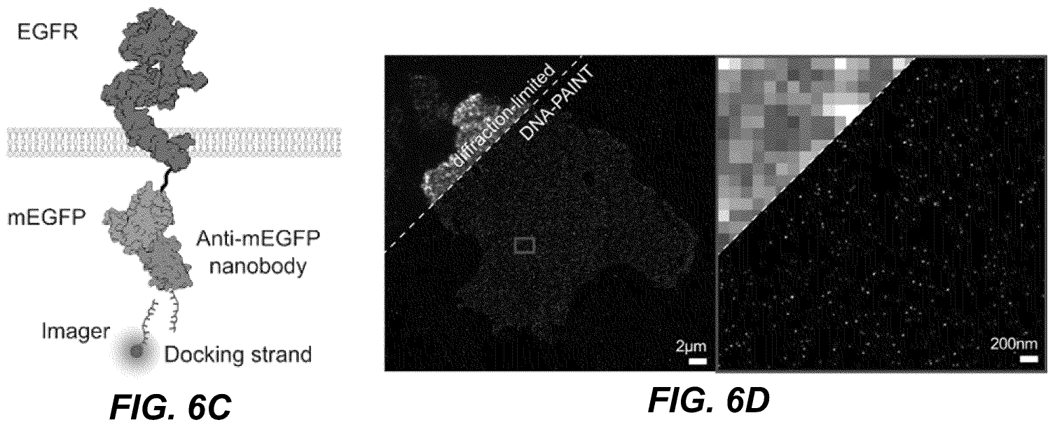

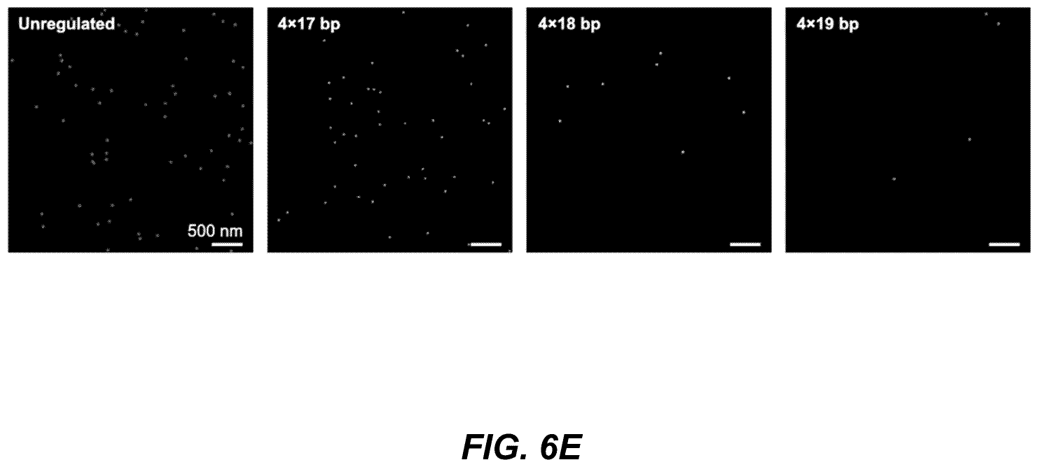

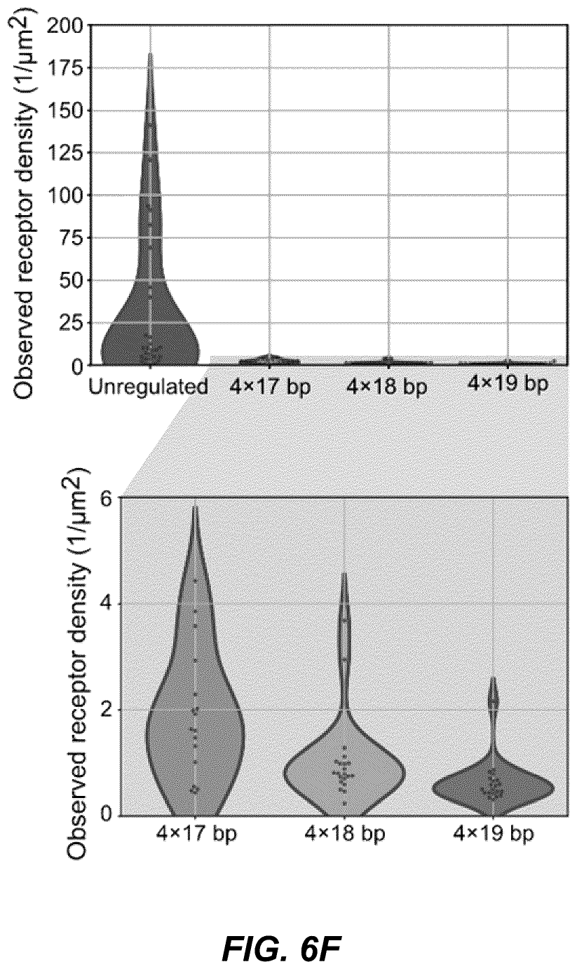

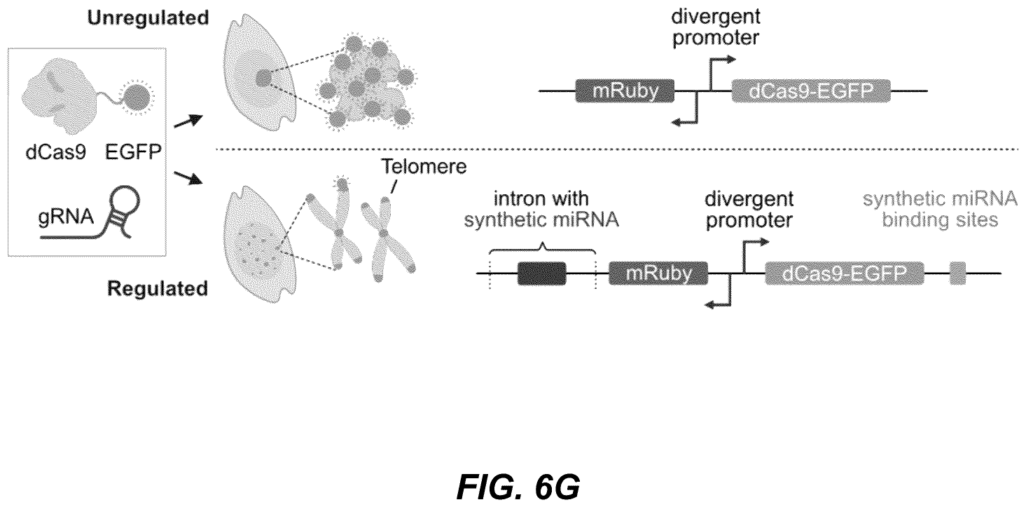

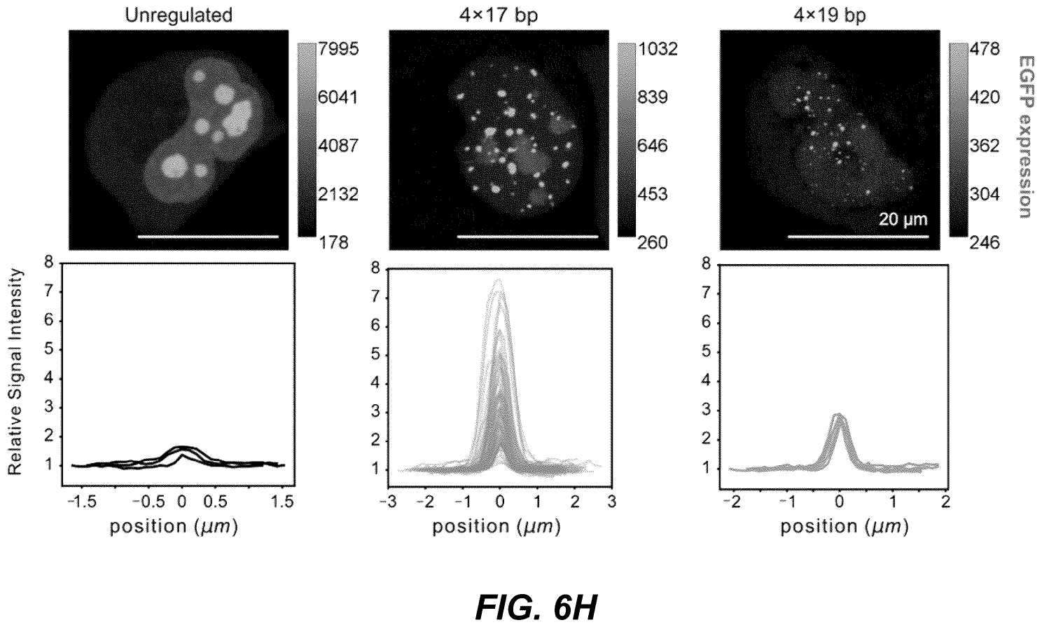

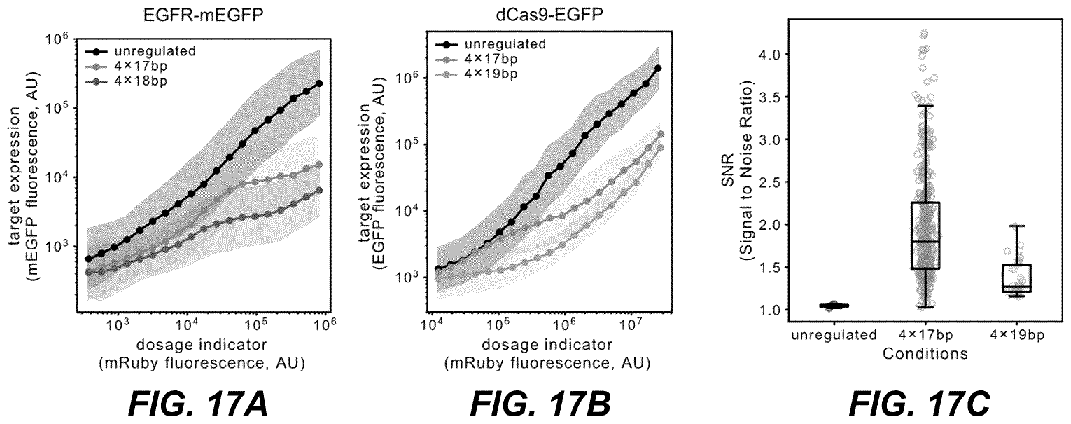

Dosage compensation can have diverse applications across biology. In quantitative imaging, limiting the expression of fluorescent proteins fused to other proteins of interest could reduce background fluorescence and allow more precise spatial localization of fusion protein distributions, and make the exogenous protein expressions matching the endogenous expressions, facilitating single-molecule imaging, even with transient transfection. To test this, an EGFR-mEGFP membrane marker fusion protein expression construct was transfected, either unregulated or controlled by the 4×17 or 4×18 circuit (FIG. 6A). Both circuits limited expression, with 4×18 providing the lowest expression levels (FIG. 17A). Further, the unregulated control showed heterogeneous expression, with ˜20% of the cells in the field overexpressing EGFR, making its membrane localization difficult to perceive (FIG. 6B, left). By contrast, in the 4×18 bp DIMMER system, most cells exhibited a more clearly defined pattern, with strong plasma membrane localization (FIG. 6B, right).

The advantage of dosage compensated expression can be observed directly and quantitatively with super-resolution imaging using DNA-PAINT (Point Accumulation for Imaging in Nanoscale Topography), which allows analysis of single protein molecules on the surface of cells. CHO-K1 cells were transfected with unregulated and regulated EGFR-mEGFP plasmids. After transient transfection for 48 hours, cells were fixed and prepared for DNA-PAINT image acquisition using DNA-conjugated anti-GFP nanobodies targeting the intracellular mEGFP tag of the EGFR receptor (FIG. 6C). Raw DNA-PAINT localizations (FIG. 6D) were subjected to recently developed clustering procedures to yield the positions of single EGF receptor proteins in the cell membrane (FIG. 6E). From these datasets, the overall receptor density for different plasmid constructs was determined.

As expected, the receptor densities from unregulated constructs were significantly higher than those of the regulated plasmids (A second design feature of these circuits is the separation of miRNA and target 6F). Specifically, the receptor density for the unregulated plasmid was 29±40 μm² (mean±s.d.), while the densities for the receptors in the cells transfected with the regulated plasmids were ˜15 times lower (4×17 bp: 1.9±1.2 μm², 4×18 bp: 1.0±0.8 μm⁻², 4×19 bp: 0.6±0.4 μm²). Excitingly, not only did the mean densities follow the expected trend, but distributions were considerably narrower for the regulated plasmids as compared to the unregulated ones (FIG. 6F).

Dosage compensation circuits also improved CRISPR imaging. For example, it is possible to image telomeres by expressing dCas9-EGFP, with or without dosage compensation, along with a gRNA targeted to repetitive telomeric sequences (FIG. 6G). Flow cytometry showed that circuits reduce dCas9-EGFP expression, and its dosage sensitivity, although not to the extent seen with other genes (FIG. 17B). With the unregulated system, dCas9-EGFP seldom labeled the telomeres but rather formed bright aggregations in the nucleolus, consistent with previous observations (FIG. 6H, left panel). By contrast, the 4×17 bp circuit restricted most fluorescence to dots, consistent with telomeric labeling, with reduced labeling of the nucleolus. Further, the stronger 4×19 bp circuit removed nearly all labeling in the nucleolus, while maintaining apparent telomere labeling. The circuits thus improved signal-to-background ratio, as evident in line scans of the images (FIG. 6H, middle and right panel) as well as contrast in each dot. Taken together, these results demonstrate how the dosage compensation circuits developed here improve imaging of proteins and cellular structures.

### DISCUSSION

Ectopic gene expression is a cornerstone of modern biology, as well as gene and cell therapy, but most approaches produce variable and dosage-sensitive expression distributions. Here, a set of miRNA-based circuits were enginerred that allow more precise, and gene dosage-invariant, control of protein expression. These circuits also allow tuning of expression level through sequence (FIGS. 2-3), orthogonal control of multiple target genes (FIG. 4), and portability across cell types and modes of delivery (FIG. 5). Accordingly, the circuits provided herein can become standard systems for controlled gene expression in diverse areas of biomedical science and biotechnology, including imaging (FIG. 6).

The process of engineering robust dosage invariant circuits required identifying design principles for miRNA regulation. The fully complementary single target site produced an extremely low setpoint of expression (FIG. 2C). Reducing complementarity is believed to tune the binding affinity between the miRNA and the target, and also the inhibition strength, therefore could have raised the expression level of the circuit. However, systematic variation of single target site complementarity showed that reducing complementarity removed regulation altogether (FIG. 2D). Remarkably, however, TNRC6-dependent cooperativity among multiple target sites can restore regulation to sites that were individually weak, and implement dosage invariance (FIG. 2E). Thus, while cooperativity in other regulatory systems is associated with ultrasensitivity, here it allows linear dependence of target expression on miRNA levels, which is required for dosage invariance (See Supplementary Modeling below). These results suggest that weak, multivalent miRNA regulation, possibly by avoiding direct cleavage, can provide the linear responses required for effective dosage compensation. It is also noted that the behavior observed here is broadly consistent with evidence for an important role for weak cooperative interactions in natural miRNA regulatory systems.

It was found that varying miRNA regulation strength by complementarity was straightforward for one case of miR-L, but was more complex for other synmiRs generated, indicating that additional factors besides base pairing are important for determining the activity of a miRNA on its target. In some embodiments, without being bound by any particular theory, the molecular regulatory mechanism in the dosage compensated regime is catalytic, and possibly slicer-independent.

A second design feature of these circuits is the separation of miRNA and target gene into separate, divergently transcribed genes. Early designs incorporating the miRNA and target into a single transcript failed to achieve full dosage invariance (FIG. 8). The divergent design allows strong expression of the miRNA and avoids the miRNA effectively inhibiting its own production. Further engineering may allow even more compact designs.

Provided herein are empirical rules that are effective for designing orthogonal miRNA sequences. In some embodiments, while circuits achieve dosage compensation across multiple cell lines (FIG. 5A, FIG. 15), total protein levels vary. Regulation of translation or protein degradation can achieve cell type independent control of protein concentration in some embodiments. In some embodiments, the use of these systems comprise modulating expression with inducers.

The DIMMER circuits provided here represent a powerful toolbox for researchers and biomedical engineers. They can reduce background in imaging (FIG. 6), can allow probing of concentration dependent effects in numerous studies, can ensure a fixed expression level for receptors or other components in cell therapies, and can also help make ectopic transcription factor expression more precise in regenerative medicine. One major application category is gene therapy. Many diseases that could be targeted by gene therapy exhibit toxicity at high levels of the therapeutic gene, making it critical to suppress overexpression. Thus, these systems can be useful components in a wide range of engineered research and therapeutic contexts.

### Methods

### Supplementary Modeling

### Plasmids Construction

Constructs used in this study are listed in Table 1. Some constructs were generated using standard cloning procedures. The inserts were generated using PCR or gBlock synthesis (IDT) and were ligated either by T4 ligase (NEB #M0202M) or In-Fusion (Takara #102518) assembly with backbones that are linearized using restriction digestion. The rest of the constructs were designed by the authors and synthesized by GenScript. Selected constructs will be deposited at Addgene and the maps are available.

miRNA Alignment to the Database

Each synthetic miRNA sequence (mature miRNA, 22 nt) is aligned to the known miRNA sequence database (mirbase.org) to identify if there are any similarities existing between the synthetic sequences and the natural sequences.

### Tissue Culture

U2OS cells, T-Rex cells, CHO cells, and N2A cells were cultured at 37° C. in a humidity-controlled chamber with 5% CO2. The growth media consisted of DMEM (Dulbecco's Modified Eagle Medium, ThermoFisher #11960-069) supplemented with 10% FBS, 1 U/ml penicillin, 1 μg/ml streptomycin, 1 mM sodium pyruvate, 1×NEAA (ThermoFisher #11140-050), 1 mM L-glutamine, and 0.1 mg/mL Normocin (InvivoGen #ant-nr).

### Transient Transfection

Cells were seeded at a density of 50,000 cells in each well of a 24 well plate, either standard for flow cytometry or glass-bottom for imaging experiments, and cultured under standard conditions overnight. The following day, the cells were transiently transfected using Fugene HD (Promega #E2311), according to the manufacturer's protocol.

### Flow Cytometry

Cells were incubated 2 days after transient transfection, and the culture media was replaced 24 hours post-transfection. Cells were trypsinized with 75 μL of 0.25% trypsin for 5 minutes at 37 C. After digestion, cells were resuspended with 125 μL of HBSS containing 2.5 mg/ml BSA and 1 mM EDTA. Cells were then filtered through a 40 μm cell strainer and analyzed using a CytoFLEX S instrument (Beckman Coulter). The self-build python code was used to analyze the flow data.

### Cell Sorting

To prepare the mono-clones that expressed the genomic-integrated DIMMER circuit, cells were harvested and resuspended in sorting buffer (BD FACS Pre-Sort Buffer) supplemented with 1 U/ml DNAse I by the cell sorter (Sony MA900) as mono-clones. Cells were sorted into 96 well plates in the normal U2OS culture media. Cells were expanded in the 24 well plate before measurement using the flow cytometer.

### In-Vitro Image Analysis

The transiently transfected U2OS cells were imaged using a Nikon confocal microscope at 60× magnification, such that each image was spaced by 0.5 microns in the z-direction. Images were processed by the Fiji software.

To analyze the relative signal intensity in the dCas9 imaging experiment, maximum intensity projection of 11 slices of the z-stacks were applied. To determine the signal intensity of the dots, freehand lines were drawn to select the dot regions. To determine the signal intensity in the background, ˜5 micron-long straight lines centering the dots were drawn. The signal intensities were generated by the Fiji ROI mean intensity function. The relative signal intensity is calculated by normalizing the background intensity to be 1.

To analyze the signal to noise ratio (SNR), freehand lines were drawn to select the dot regions and the nucleus regions. The noise intensity was calculated by the intensity in the nucleus excluding the dots area. The SNR was calculated dividing the mean intensities of each dot by the noise.

### Bulk RNA Sequencing

### Sample Preparation and Sequencing

To verify the off-target effect of all the synmiRs, U2OS cells were plated on 6-well plates with 300,000 cells per well. Cells were transfected the following day with 1,000 ng of either the control plasmid or the BFP-miRNA plasmid using Fugene HD (Promega #E2311) according to the manufacturer's instructions. Media was replaced with 2 mL of fresh media 24 hours post-transfection. Cells were harvested 48 hours post-transfection by digestion with 0.25% Trypsin-EDTA, centrifugation at 300 g for 5 minutes, and removal of the supernatant by aspiration. The cell's pellet was stored in −80 C prior to the purification.

RNA was extracted using the RNeasy kit (Qiagen #74106) according to the manufacturer's instructions. RNA was treated with Turbo DNase (ThermoFisher #AM2238) and purified using the RNeasy kit RNA cleanup protocol. mRNA sequencing libraries were prepared by Novogene.

### Preprocessing of Sequencing Data

Reads from the RNA sequencing were aligned to a custom reference genome using kallisto (0.48.0). This reference consisted of the human genome GRCh38 cDNA and mTagBFP2 coding sequences. Weakly expressed genes were filtered out if they exhibited fewer than 3 samples expressing at least 10 transcripts per million (TPM), or if the maximum TPM among all samples was less than 105. Then, filtered counts were input to DEseq to eliminate the impact of size factors. As the BFP-only cells were used as a reference to evaluate the off-target effect of the miRNAs, genes that showed fluctuating expressions among the three biological replicates of BFP-only cells should be removed from analysis. To achieve this, log(1+x) was computed, where x denotes the normalized TPM among the three biological replicates of BFP-only cells. The Fano factors of these logarithmic expressions were determined and ranked. Transcripts that ranked as the largest 2.5% in logarithmic Fano factors were eliminated from further analysis. Finally, log(1+x) was computed, where x denotes the normalized TPM among all the samples. A difference function was defined to compute the absolute value of the log(1+x) difference between each sample and the non-transfected sample. The medians of the difference function of the BFP-only groups and the experimental groups were calculated and used for comparison. The difference between those two difference functions were ranked and similarly, transcripts that ranked as the largest 3% were removed from further analysis.

The Fano factor is defined as:

$$Fano factor = \frac{variance ( log ( 1 + x ) )}{mean ( log ( 1 + x ) )}$$

The equation of the difference function is defined as:

$$Δ ( BFP , untransfected ) = \vert log ( 1 + {x}_{BFP} ) - log ( 1 + {x}_{untransfected} ) \vert$$

$$Δ ( experimental , untransfected ) = \vert log ( 1 + {x}_{experimental} ) - log ( 1 + {x}_{untransfected} ) \vert$$

$${Δ}_{ranked} = median ( Δ ( BFP , untransfected ) ) - median ( Δ ( experimental , untransfected ) )$$

### Differential Gene Expression Analysis

To characterize the perturbations that synthetic miRNA brought to the endogenous transcriptome, differential expression analysis was performed using DESeq2 (1.40.1) in R (4.3.1) comparing transcript counts in miRNA transfected cells and BFP-only cells.

### DNA-PAINT

### Buffers

Blocking buffer: 1×PBS, 1 mM EDTA (Thermo Fisher, no. AM9260G), 0.02% Tween-20 (Life Science, no. P7949), 2% BSA (Sigma-Aldrich, no. A9647-100G)
Washing buffer: 1×PBS, 1 mM EDTA, 0.01% Tween-20
Imaging buffer: 1×PBS, 1 mM EDTA, 500 mM NaCl (Thermo Fisher, no. AM9760G)

### Cloning

An mEGFP Gblock (obtained from Integrated DNA Technologies) was inserted into a pcDNA3.1(+) backbone (ThermoFisher, no. V79020) via Gibson assembly. Two codon-optimized fragments of human EGFR (obtained from Integrated DNA Technologies) were fused to the mEGFP-pcDNA3.1(+) backbone via Gibson assembly.

### Cell Culture

CHO-K1 cells (ATCC: CCL-61) were cultured in Gibco Ham's F-12K (Kaighn's) medium supplemented with 10% FBS (Gibco, no. 11573397) at 37° C. and 4% CO2. Cells were split via trypsinization using trypsin-EDTA (Gibco, no. 25300096) every 2-3 days.

### Nanobody-DNA Conjugation

First, the GFP nanobody (clone 1H1, Nanotag Biotechnologies) was conjugated to a DBCO-PEG4-Maleimide linker (no. CLK-A108P, Jena Bioscience). After removing unreacted linker with Amicon centrifugal filters (10,000 MWCO), the DBCO-nanobody was conjugated via DBCO-azide click chemistry to the docking strand 5′-azide-CTC TCT CTC TCT CTC TCT C-3′ (Metabion). A detailed description of the conjugation can be found in a previous protocol provided in Strauss, S. & Jungmann, R. (Up to 100-fold speed-up and multiplexing in optimized DNA-PAINT. Nat. Methods 17, 789-791 (2020).

### DNA-PAINT Imaging

5,000 cm-2 CHO-K1 cells were seeded on an ibidi eight-well high glass-bottom chambers (no. 80807) one day before transfection. The cells were transfected with EGFR-mEGFP plasmids with a ThermoFisher Lipofectamine 3,000 reagent (no. L3000008) with a lower Lipofectamine concentration as indicated by the manufacturer and 250 ng plasmid per well (200 μL solution per well and 25 μL transfection solution). After 48 h of transfection, the cells were fixed with 250 μL of pre-warmed methanol-free 4% PFA (ThermoFisher, no. 043368.9M) in 1×PBS for 15 min. The cells were washed 3 times with 1×PBS and then permeabilized with 0.125% TritonX-100 (Sigma Aldrich, no. 93443) in 1×PBS for 2 min. After washing 3 times with 1×PBS, the cells were blocked with the blocking buffer overnight at 4° C.

The cells were then washed three times with the washing buffer. 25 nM anti-GFP nanobodies were incubated in the blocking buffer for 1 h at RT. After washing 3 times with the washing buffer, the sample was incubated with the imaging buffer for 5 min. The nanobodies were then post-fixed with 4% PFA in 1×PBS for 5 min. The cells were then washed 3 times with the washing buffer and once with 1×PBS. 90 nm gold-nanoparticles (Absource, no. G-90-100) in 1:1 in 1×PBS were incubated for 5 min at RT. After washing three times with 1×PBS, the cells were washed once with the imaging buffer. The samples were imaged in the imaging buffer with 100 pM imager strand (5′-GAG AGA G-Cy3B 3′, obtained from Metabion) for 40k frames with 100 ms exposure time per frame and a readout rate of 200 MHz.

### Microscope Setup

The samples were measured on inverted total internal reflection fluorescence (TIRF) microscopes (Nikon Instruments, Eclipse Ti2) which are equipped with an oil-immersion objective (Nikon Instruments, Apo SR TIRF×100/numerical aperture 1.49, oil) and a perfect focusing system. The mRuby3 signal was bleached by the 560 nm laser (MPB Communications, 1 W) by using Highly inclined and laminated optical sheet (HILO) illumination. Afterwards, the TIRF mode was established. The Cy3B imagers were excited with the 560 nm laser. The laser beam was cleaned with a filter (Chroma Technology, no. ZET561/10) and coupled into the microscope with abeam splitter (Chroma Technology, no. ZT561rdc). The fluorescent signal was filtered with an emission filter (Chroma Technology, nos. ET600/50m and ET575lp) and projected onto a sCMOS camera (Hamamatsu, ORCA-Fusion BT) without further magnification.

The acquired region of interest has a size of 576×576 pixels. The resulting effective pixel size is 130 nm. The raw microscopy data was acquired via pManager.

### DNA-PAINT Analysis

Obtained fluorescent data was reconstructed with Picasso software. The data was first drift-corrected with redundant cross-correlation, after that with picked gold particles as fiducials. In order to determine the receptor density, a homogeneous area of the cells was picked and the DNA-PAINT data was clustered with the SMLMS clustering algorithm of Picasso. The determined cluster centers were used to calculate the receptor density per μm² (number of cluster centers per area).

### Supplementary Modeling

1 Modeling miRNA-Based Gene Dosage Compensation Circuits

Herein is developed a simplified mathematical model of miRNA-based incoherent feed-forward loop (IFFL) circuits, and it is used to explore how various parameters control the scaling of target gene expression with gene dosage. The model is based on several reactions:

1. RISC production and removal. It is assumed that miRNA is expressed, processed, and assembles with Argonaute (Ago) proteins to form an active (mature) RNA-Induced Silencing Complex (RISC). The concentration of mature RISC (containing the miRNA) is denoted as r. It is assumed that RISC is produced at a total rate of Dβᵣ, where D denotes gene copy number (gene dosage) and βᵣ denotes the rate of production RISC production per gene copy. This expression implicitly assumes that miRNA expression levels do not saturate available miRNA processing machinery, Ago, or other components. It is also assumed that the RISC complex is removed at total rate γᵣr, where γᵣ denotes a combined rate constant for dilution, degradation, and other removal processes.

2. The mRNA of the target gene, whose concentration is denoted m, similarly undergoes production and removal. It is assumed that mRNA is produced at a rate proportional to gene copy number, Dβₘ, where βₘ is the mRNA production rate per gene copy. mRNA can also be removed at a rate γₘm due to dilution and degradation. It is noted that even though mRNA and miRNA are produced from the same engineered locus, the production rate constants βₘ and βᵣ can differ, since the miRNA and mRNA are produced from distinct promoters, and because mRNA and RISC-miRNA complex production involve distinct biochemical steps.

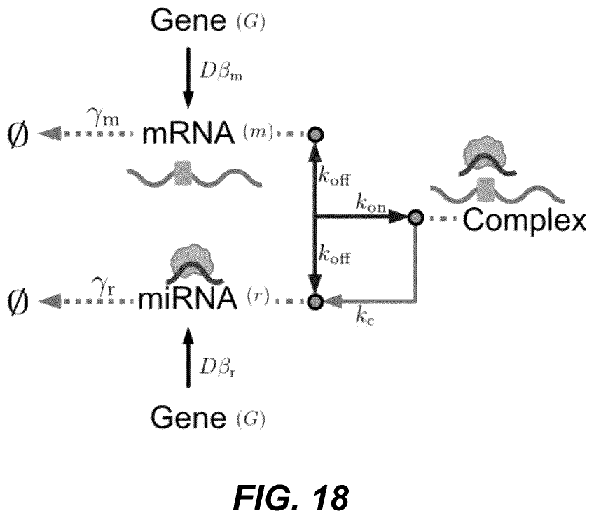

3. RISC-mRNA complex formation and dissociation. It is assumed that the RISC and target mRNA associate to form a complex, whose concentration is denoted C, at a rate kₒₙrm, following mass action kinetics with rate constant, kₒₙ. Once formed, this complex can dissociate at rate k_{off} C, undergo catalytic mRNA degradation, at rate k_{c}C. FIG. 18 depicts a non-limiting exemplary schematic of reactions in model.

These chemical reactions can be summarized as follows, using G to denote the gene, present at copy number (dosage) D:

$$G {D {β}_{r}}_{\to } G + r$$

$$r {{γ}_{r}}_{\to } ∅$$

$$G {D {β}_{m}}_{\to } G + m$$

$$m {{γ}_{m}}_{\to } ∅$$

$$r + m {{k}_{on}}_{\to } C$$

$$C {{k}_{off}}_{\to } r + m$$

$$C {{k}_{c}}_{\to } r$$

With these definitions and assumptions, one can write down a set of ordinary differential equations for the three variables, r, m, and C.

$$\begin{matrix}\frac{dr}{dt} = {β}_{r} D - {γ}_{r} r - {k}_{on} rm + ( {k}_{c} + {k}_{off} ) C \\ \frac{d m}{dt} = {β}_{m} D - {γ}_{m} m - {k}_{on} rm + {k}_{off} C \\ \frac{dC}{dt} = {k}_{on} rm - ( {k}_{c} + {k}_{off} ) C\end{matrix}$$

These equations resemble previous modeling of regulation by small RNAs, except for the explicit incorporation of gene dosage in the control of both mRNA and miRNA.

To gain insight into the possible behaviors of this system, one can first non-dimensionalize it. One can define a dimensionless time, \tilde{t}=γᵣt, by rescaling time in units of the RISC lifetime. One can also define a dimensionless RISC concentration, \tilde{r}=r/(βᵣ/γᵣ). This effectively rescales r in units of the unregulated steady-state expression level produced by a single copy of the gene. One can similarly define a dimensionless mRNA concentration, \tilde{m}−=m/(βₘ/γₘ), normalizing m by its single copy steady state expression level. Finally, one can define a dimensionless concentration of the RISC-miRNA-mRNA complex, \tilde{C}=C/(βₘβᵣ/γₘγᵣ).

In addition, one can define a set of convenient dimensionless parameter ratios:

$$K = \frac{{k}_{c} + {k}_{off}}{{k}_{on}}$$

$$γ = \frac{{γ}_{m}}{{γ}_{r}}$$

$$β = \frac{{β}_{m}}{{β}_{r}}$$

$${\tilde{k}}_{on} = \frac{{k}_{on} {β}_{r}}{{γ}_{m} {γ}_{r}}$$

$${\tilde{k}}_{off} = \frac{{k}_{off} {β}_{r}}{{γ}_{m} {γ}_{r}}$$

$${\tilde{k}}_{c} = \frac{{k}_{c} {β}_{r}}{{γ}_{m} {γ}_{r}}$$

In the non-dimensionalized system, the differential equations can be written as,

$$\begin{matrix}\frac{d \tilde{r}}{d \tilde{t}} = D - \tilde{r} - {\tilde{k}}_{on} β \tilde{m} \tilde{r} + ( {\tilde{k}}_{c} + {\tilde{k}}_{off} ) β \tilde{C} \\ {γ}^{- 1} \frac{d \tilde{m}}{d \tilde{t}} = D - \tilde{m} - {\tilde{k}}_{on} \tilde{m} \tilde{r} + {\tilde{k}}_{off} \tilde{C} \\ {γ}^{- 1} \frac{d \tilde{C}}{d \tilde{t}} = \frac{{γ}_{r}}{{β}_{r}} {\tilde{k}}_{on} ( \tilde{m} \tilde{r} - K \tilde{C} )\end{matrix}$$

By definition, at steady state, the time derivatives all equal zero. Denoting steady values with a subscript s, one then has:

$$\begin{matrix}D - {\tilde{r}}_{s} - {\tilde{k}}_{on} β {\tilde{m}}_{s} {\tilde{r}}_{s} + ( {\tilde{k}}_{c} + {\tilde{k}}_{off} ) β \tilde{{C}_{s}} = 0 \\ D - {\tilde{m}}_{s} - {\tilde{k}}_{on} {\tilde{m}}_{s} {\tilde{r}}_{s} + {\tilde{k}}_{off} \tilde{{C}_{s}} = 0 \\ {\tilde{m}}_{s} {\tilde{r}}_{s} - K \tilde{{C}_{s}} = 0\end{matrix}$$

Solving the equations above, one obtains an equation for steady-state mRNA concentration:

$${\tilde{m}}_{s} = \frac{D}{1 + \frac{{\tilde{k}}_{c}}{K} D}$$

Henceforth, the tildes are omitted for notational convenience, and switch to the non-dimensionalized variables and parameters.

In the limit of large dosage,

$$\frac{{k}_{c}}{K} D >> 1.$$

As a result, mₛ approaches a limiting value,

$${m}_{s} \to \frac{K}{{k}_{c}}$$

independent of gene dosage. This is the regime that the constructs developed here are targeting.

FIG. 2A plots this expression, and FIGS. 7A-7B plot the expression in different parameters, i.e. k_{off}, and k_{c}.

Finally, to estimate biological parameter values, it was assumed that the miRNA and mRNA were produced at similar rates, and selected values based on [Nyayanit, D. & Gadgil, C. J. Mathematical modeling of combinatorial regulation suggests that apparent positive regulation of targets by miRNA could be an artifact resulting from competition for mRNA. Rna 21, 307-319 (2015)], as listed in Table 9 below.

TABLE 9
Parameters

| Non-dimensional parameter | Dimensionless values |
|---|---|
| β | 1 |
| kₒₙ | 200000 |
| k_{off} | 4000, 40000, 400000, 4000000 |
| k^{c} | 160, 1.6, 0.16 |

### 1.1 Incorporating Ultrasensitivity

Alternatively, a more general, phenomenological model was considered in which the inhibition of miRNA to the target was assumed to follow a Hill function, with the Hill coefficient n. This treatment omits intermediate steps, i.e., the RISC-mRNA complex formation and dissociation. With this assumption, one can write down a different set of ordinary differential equations for the two variables, r, and m.

$$\begin{matrix}\frac{dr}{dt} = {β}_{r} D - {γ}_{r} r \\ \frac{dm}{dt} = \frac{{β}_{m} D}{1 + {( \frac{r}{\text{?}} )}^{n}} - {γ}_{m} m\end{matrix}$$

$$\text{?} \text{indicates text missing or illegible when filed}$$

One can non-dimensionalize this system by defining \tilde{t}=γᵣt, \tilde{r}=r/(βᵣ/γᵣ), and \tilde{m}=m/(βₘ/γₘ). Additionally, one can define

$$\tilde{K} = \frac{{β}_{r}}{{γ}_{r} \text{?}}$$

$$\text{?} \text{indicates text missing or illegible when filed}$$

for convenience. The steady state expression of mRNA concentration can then be written as:

$$= \frac{D}{1 + {( \tilde{K} D )}^{n}}$$

FIG. 7C plots the expression when \tilde{K} is set as 1, and n=0.5, 1, 2. Critically, only when n=1, does \tilde{m}ₛ approaches a limiting value. If n<1, \tilde{m}ₛ shows a sublinear increase with D. If n>1, \tilde{m}ₛ shows a bandpass filter dependence with D.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nucleic acid composition, comprising:
a first promoter sequence operably linked to a first polynucleotide comprising one or more miRNA cassettes,
wherein the first promoter sequence is capable of inducing transcription of the first polynucleotide to generate a first transcript,
wherein the first transcript is capable of being processed to generate said miRNA; and
a second promoter sequence operably linked to a second polynucleotide comprising a payload gene,
wherein the payload gene comprises a miRNA target region comprising one or more miRNA target sequences,
wherein the second promoter sequence is capable of inducing transcription of the second polynucleotide to generate a payload transcript, and
wherein the payload transcript is capable of being translated to generate a payload protein.

2. A composition, comprising:
one or more cells comprising:
a first promoter sequence operably linked to a first polynucleotide comprising one or more miRNA cassettes,
wherein the first promoter sequence is capable of inducing transcription of the first polynucleotide to generate a first transcript,
wherein the first transcript is capable of being processed to generate said miRNA; and
a second promoter sequence operably linked to a second polynucleotide comprising a payload gene,
wherein the payload gene comprises a miRNA target region comprising one or more miRNA target sequences,
wherein the second promoter sequence is capable of inducing transcription of the second polynucleotide to generate a payload transcript, and
wherein the payload transcript is capable of being translated to generate a payload protein.

3. The nucleic acid composition of claim 1,
wherein the first promoter sequence and the second promoter sequence are components of a bidirectional promoter, and
wherein the first promoter sequence and the second promoter sequence are in reverse complementary orientation with respect to each other in the bidirectional promoter.

4. The nucleic acid composition of claim 1, wherein the miRNA is capable of binding the one or more miRNA target sequences, thereby reducing the stability of the payload transcript and/or reducing the translation of the payload transcript.

5. The nucleic acid composition of claim 1,
wherein the first polynucleotide comprises a dosage gene;
wherein the first transcript is capable of being translated to generate dosage indicator protein;
wherein an intron is located in the dosage gene 3′UTR, dosage gene 5′UTR, or between dosage gene exons; and
wherein the intron comprises the one or more miRNA cassettes.

6. The nucleic acid composition of claim 1, wherein, in a cell comprising the nucleic acid composition, the payload protein reaches tuned steady state payload protein levels, wherein tuned steady state payload protein levels range between a lower tuned threshold and an upper tuned threshold of a tuned expression range, wherein the difference between the lower tuned threshold and the upper tuned threshold of the tuned expression range is less than about one order of magnitude.

7. The nucleic acid composition of claim 6, wherein the lower tuned threshold and/or the upper tuned threshold of a tuned expression range is capable of being configured by modulating one or more of (i) the number of miRNA cassettes within the first polynucleotide, (ii) the number of miRNA target sequences in the miRNA target region, (iii) the complementarity between the miRNA and the one or more miRNA target sequences, and (iv) strength of the first promoter sequence and/or second promoter sequence.

8. The nucleic acid composition of claim 6, wherein the payload protein is efficacious at steady state payload protein levels within the tuned expression range, and wherein the payload protein is inefficacious and/or toxic at steady state payload protein levels above and/or below the tuned expression range.

9. The nucleic acid composition of claim 1, wherein the miRNA comprises a nucleotide sequence that is at least 80%, 85%, 90%, 95%, 98%, 99%, or 100% identical to SEQ ID NOs: 75-87.

10. The nucleic acid composition of claim 1, wherein the miRNA cassette comprises a nucleotide sequence that is at least 80%, 85%, 90%, 95%, 98%, 99%, or 100% identical to SEQ ID NOs: 1-13.

11. The nucleic acid composition of claim 1, wherein the miRNA target region comprises a nucleotide sequence that is at least 80%, 85%, 90%, 95%, 98%, 99%, or 100% identical to SEQ ID NOs: 14-74.

12. The nucleic acid composition of claim 1, wherein the miRNA target region comprises at least about 1, 2, 3, 4, 5, 6, 7, 8, 9, 10, 11, 12, 13, 14, 15, 16, 17, 18, 19, or 20 miRNA target sequences.

13. The nucleic acid composition of claim 1, wherein 5, 6, 7, 8, 9, 10, 11, 12, 13, 14, 15, 16, 17, 18, 19, 20, 21, nucleotides (nt) of a miRNA target sequence is complementary to the miRNA.

14. The nucleic acid composition of claim 1, wherein the miRNA comprises 5-8 GC nucleotides.

15. The nucleic acid composition of claim 1, comprising one or more supplemental payload genes and one or more supplemental miRNA, wherein the supplemental miRNA differ in sequence with respect to each other, and wherein the nucleic acid composition, for each distinct supplemental miRNA, comprises:
a supplemental first promoter sequence operably linked to a supplemental first polynucleotide comprising one or more supplemental miRNA cassettes,
wherein the supplemental first promoter sequence is capable of inducing transcription of the supplemental first polynucleotide to generate a supplemental first transcript,
wherein the supplemental first transcript is capable of being processed to generate said supplemental miRNA; and
a supplemental second promoter sequence operably linked to a supplemental second polynucleotide comprising a supplemental payload gene,
wherein the supplemental payload gene comprises a supplemental miRNA target region comprising one or more supplemental miRNA target sequences,
wherein the supplemental second promoter sequence is capable of inducing transcription of the supplemental second polynucleotide to generate a supplemental payload transcript,
wherein the supplemental payload transcript is capable of being translated to generate a supplemental payload protein.

16. The nucleic acid composition of claim 1,
wherein the first promoter sequence and/or second promoter sequence comprises a ubiquitous promoter selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof;
wherein the first promoter sequence and/or second promoter sequence is an inducible promoter selected from the group comprising a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter;
wherein the first promoter sequence and/or second promoter sequence comprises a tissue-specific promoter and/or a lineage-specific promoter;
wherein the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter;
wherein the tissue specific promoter is a neuron-specific promoter selected from the group comprising a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ1.7 promoter, a Naᵥ1.8 promoter, a Naᵥ1.9 promoter, or an Advillin promoter;
wherein the tissue specific promoter is a muscle-specific promoter;
wherein the the first promoter sequence and/or second promoter sequence is a methyl CpG binding protein 2 (MeCP2) promoter or a derivative thereof; and/or
wherein one or more cells comprise an endogenous version of the payload gene, and wherein the promoter comprises or is derived from the promoter of the endogenous version.

17. The nucleic acid composition of claim 1, wherein a payload protein comprises:
a disease-associated protein, wherein aberrant expression of the disease-associated protein correlates with the occurrence and/or progression of the disease;
a protein associated with an expression-sensitive disease or disorder;
methyl CpG binding protein 2 (MeCP2), SMN, DRK1A, KAT6A, NIPBL, HDAC4, UBE3A, EHMT1, one or more genes encoded on chromosome 9q34.3, NPHP1, LIMK1 one or more genes encoded on chromosome 711.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof;
a component of a RNA export system, a lipid-enveloped nanoparticle (LN) production system, or a virus-like particle (VLP) production system;
an imaging agent selected from the group comprising green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof;
an endogenous protein or exogenous protein associated with a binding domain; and/or
a programmable nuclease selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions.

18. The nucleic acid composition of claim 1, wherein a payload protein comprises:
a chimeric antigen receptor (CAR) or T-cell receptor (TCR);
a CRE recombinase, GCaMP, a cell therapy component, a knock-down gene therapy component, a cell-surface exposed epitope, or any combination thereof;
a bispecific T cell engager (BiTE);
a cytokine selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof;
a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5;
a member of the TNF family of cytokines selected from the group consisting of TNF-alpha, TNF-beta, LT-beta, CD40 ligand, Fas ligand, CD 27 ligand, CD 30 ligand, and 4-1 BBL;
a member of the immunoglobulin superfamily of cytokines selected from the group consisting of B7.1 (CD80) and B7.2 (B70);
an interferon selected from interferon alpha, interferon beta, or interferon gamma;
a chemokine selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15;
an interleukin selected from IL-10 IL-12, IL-1, IL-6, IL-7, IL-15, IL-2, IL-18 or IL-21;
a tumor necrosis factor (TNF) selected from TNF-alpha, TNF-beta, TNF-gamma, CD252, CD154, CD178, CD70, CD153, or 4-1BBL; and/or
a factor locally down-regulating the activity of endogenous immune cells.

19. The nucleic acid composition of claim 1, wherein the nucleic acid composition is, comprises, or further comprises, one or more vectors,
wherein at least one of the one or more vectors is a viral vector, a plasmid, a transposable element, a naked DNA vector, a lipid nanoparticle (LNP), or any combination thereof,
wherein the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof, and
wherein the transposable element is piggybac transposon or sleeping beauty transposon.

20. A method of treating a disease or disorder in a subject, the method comprising:
administering to the subject an effective amount of the nucleic acid composition of claim 1, thereby treating or preventing the disease or disorder in the subject.


SEQUENCE LISTING (87 sequences)

SEQ ID NO: 1 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgccaacataagcataaactacgatagtgaagccacagatgtatcgtagtttatgcttatgttgatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 2 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgctaatctacaaagtgacataaatagtgaagccacagatgtatttatgtcactttgtagattaatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 3 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgctgatctacaaagtgacataaatagtgaagccacagatgtatttatgtcactttgtagatcaatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 4 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgcgaaaagtttataatatcttgatagtgaagccacagatgtatcaagatattataaacttttcatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 5 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgcgagaagtttataatatcttgatagtgaagccacagatgtatcaagatattataaacttctcatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 6 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgccgtaaagatcatgaaattgaatagtgaagccacagatgtattcaatttcatgatctttacgatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 7 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgcagcatctatctaacggtttgatagtgaagccacagatgtatcaaaccgttagatagatgctatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 8 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgcctcttatcaagcagtttcatatagtgaagccacagatgtatatgaaactgcttgataagagatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 9 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgcagaaatggtgtaatttagcaatagtgaagccacagatgtattgctaaattacaccatttctatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 10 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgccgttgagatttaaagatcgaatagtgaagccacagatgtattcgatctttaaatctcaacgatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 11 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgccaacacctttcttaaaacttatagtgaagccacagatgtataagttttaagaaaggtgttgatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 12 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgcggtaatcttaagcatagatgatagtgaagccacagatgtatcatctatgcttaagattaccatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 13 - DNA, 348 bp, synthetic construct
  source 1..348: mol_type = other DNA
ttgaatgaggcttcagtactttacagaatcgttgcctgcacatcttggaaacacttgctgggattacttcgacttcttaacccaacagaaggctcgagaaggtatattgctgttgacagtgagcgcaggaattataatgcttatctatagtgaagccacagatgtatagataagcattataattcctatgcctactgcctcggacttcaaggggctagaattcgagcaattatcttgtttactaaaactgaataccttgctatctctttgatacatttttacaaagctgaattaaaatggtataaattaaatcacttttcctgaccattcatcctctttctttttcct

SEQ ID NO: 14 - DNA, 21 bp, synthetic construct
  source 1..21: mol_type = other DNA
aggaattataatgcttatcta

SEQ ID NO: 15 - DNA, 21 bp, synthetic construct
  source 1..21: mol_type = other DNA
tactattataatgcttatcta

SEQ ID NO: 16 - DNA, 21 bp, synthetic construct
  source 1..21: mol_type = other DNA
cttaattataatgcttatcta

SEQ ID NO: 17 - DNA, 21 bp, synthetic construct
  source 1..21: mol_type = other DNA
ttgaattataatgcttatcta

SEQ ID NO: 18 - DNA, 21 bp, synthetic construct
  source 1..21: mol_type = other DNA
tggaattataatgcttatcta

SEQ ID NO: 19 - DNA, 42 bp, synthetic construct
  source 1..42: mol_type = other DNA
tactattataatgcttatctatactattataatgcttatcta

SEQ ID NO: 20 - DNA, 63 bp, synthetic construct
  source 1..63: mol_type = other DNA
tactattataatgcttatctatactattataatgcttatctatactattataatgcttatcta

SEQ ID NO: 21 - DNA, 84 bp, synthetic construct
  source 1..84: mol_type = other DNA
tactattataatgcttatctatactattataatgcttatctatactattataatgcttatctatactattataatgcttatcta

SEQ ID NO: 22 - DNA, 114 bp, synthetic construct
  source 1..114: mol_type = other DNA
tacttggcattatcttatctatacaacaatttacttggcattatcttatctataatcacttgtacttggcattatcttatctatacaactggatacttggcattatcttatcta

SEQ ID NO: 23 - DNA, 114 bp, synthetic construct
  source 1..114: mol_type = other DNA
tacttggcattagcttatctatacaacaatttacttggcattagcttatctataatcacttgtacttggcattagcttatctatacaactggatacttggcattagcttatcta

SEQ ID NO: 24 - DNA, 114 bp, synthetic construct
  source 1..114: mol_type = other DNA
tacttggcatttgcttatctatacaacaatttacttggcatttgcttatctataatcacttgtacttggcatttgcttatctatacaactggatacttggcatttgcttatcta

SEQ ID NO: 25 - DNA, 114 bp, synthetic construct
  source 1..114: mol_type = other DNA
tacttggcatatgcttatctatacaacaatttacttggcatatgcttatctataatcacttgtacttggcatatgcttatctatacaactggatacttggcatatgcttatcta

SEQ ID NO: 26 - DNA, 114 bp, synthetic construct
  source 1..114: mol_type = other DNA
tacttggcaaatgcttatctatacaacaatttacttggcaaatgcttatctataatcacttgtacttggcaaatgcttatctatacaactggatacttggcaaatgcttatcta

SEQ ID NO: 27 - DNA, 114 bp, synthetic construct
  source 1..114: mol_type = other DNA
tacttggctaatgcttatctatacaacaatttacttggctaatgcttatctataatcacttgtacttggctaatgcttatctatacaactggatacttggctaatgcttatcta

SEQ ID NO: 28 - DNA, 84 bp, synthetic construct
  source 1..84: mol_type = other DNA
tacttggataatgcttatctatacttggataatgcttatctatacttggataatgcttatctatacttggataatgcttatcta

SEQ ID NO: 29 - DNA, 114 bp, synthetic construct
  source 1..114: mol_type = other DNA
tacttgtataatgcttatctatacaacaatttacttgtataatgcttatctataatcacttgtacttgtataatgcttatctatacaactggatacttgtataatgcttatcta

SEQ ID NO: 30 - DNA, 76 bp, synthetic construct
  source 1..76: mol_type = other DNA
ttataatgcttatctaaatattataatgcttatctaaatattataatgcttatctaaatattataatgcttatcta

SEQ ID NO: 31 - DNA, 84 bp, synthetic construct
  source 1..84: mol_type = other DNA
aattataatgcttatctaaataaattataatgcttatctaaataaattataatgcttatctaaataaattataatgcttatcta

SEQ ID NO: 32 - DNA, 88 bp, synthetic construct
  source 1..88: mol_type = other DNA
gaattataatgcttatctaaatagaattataatgcttatctaaatagaattataatgcttatctaaatagaattataatgcttatcta

SEQ ID NO: 33 - DNA, 114 bp, synthetic construct
  source 1..114: mol_type = other DNA
tggaattataatgcttatctatacaacaatttggaattataatgcttatctataatcacttgtggaattataatgcttatctatacaactggatggaattataatgcttatcta

SEQ ID NO: 34 - DNA, 84 bp, synthetic construct
  source 1..84: mol_type = other DNA
aggaattataatgcttatctaaggaattataatgcttatctaaggaattataatgcttatctaaggaattataatgcttatcta

SEQ ID NO: 35 - DNA, 243 bp, synthetic construct
  source 1..243: mol_type = other DNA
tacttggcattatcttatctatacaacaatttacttggcattatcttatctataatcacttgtacttggcattatcttatctatacaactggatacttggcattatcttatctataactagttctttgatacttggcattatcttatctatacaacaatttacttggcattatcttatctataatcacttgtacttggcattatcttatctatacaactggatacttggcattatcttatcta

SEQ ID NO: 36 - DNA, 210 bp, synthetic construct
  source 1..210: mol_type = other DNA
aggaattataatgcttatctaaggaattataatgcttatctaaggaattataatgcttatctaaggaattataatgcttatctaaggaattataatgcttatctaaggaattataatgcttatctaaggaattataatgcttatctaaggaattataatgcttatctaaggaattataatgcttatctaaggaattataatgcttatcta

SEQ ID NO: 37 - DNA, 21 bp, synthetic construct
  source 1..21: mol_type = other DNA
caacataagcataaactacga

SEQ ID NO: 38 - DNA, 21 bp, synthetic construct
  source 1..21: mol_type = other DNA
taatctacaaagtgacataaa

SEQ ID NO: 39 - DNA, 21 bp, synthetic construct
  source 1..21: mol_type = other DNA
tgatctacaaagtgacataaa

SEQ ID NO: 40 - DNA, 21 bp, synthetic construct
  source 1..21: mol_type = other DNA
gaaaagtttataatatcttga

SEQ ID NO: 41 - DNA, 21 bp, synthetic construct
  source 1..21: mol_type = other DNA
gagaagtttataatatcttga

SEQ ID NO: 42 - DNA, 21 bp, synthetic construct
  source 1..21: mol_type = other DNA
cgtaaagatcatgaaattgaa

SEQ ID NO: 43 - DNA, 21 bp, synthetic construct
  source 1..21: mol_type = other DNA
agcatctatctaacggtttga

SEQ ID NO: 44 - DNA, 21 bp, synthetic construct
  source 1..21: mol_type = other DNA
ctcttatcaagcagtttcata

SEQ ID NO: 45 - DNA, 21 bp, synthetic construct
  source 1..21: mol_type = other DNA
agaaatggtgtaatttagcaa

SEQ ID NO: 46 - DNA, 21 bp, synthetic construct
  source 1..21: mol_type = other DNA
cgttgagatttaaagatcgaa

SEQ ID NO: 47 - DNA, 21 bp, synthetic construct
  source 1..21: mol_type = other DNA
caacacctttcttaaaactta

SEQ ID NO: 48 - DNA, 21 bp, synthetic construct
  source 1..21: mol_type = other DNA
ggtaatcttaagcatagatga

SEQ ID NO: 49 - DNA, 114 bp, synthetic construct
  source 1..114: mol_type = other DNA
acaatattcgtataactacgatacaacaattacaatattcgtataactacgataatcacttgaaaatattcgtataactacgatacaactggaacaatattcgtataactacga

SEQ ID NO: 50 - DNA, 114 bp, synthetic construct
  source 1..114: mol_type = other DNA
acaatattcgtaaaactacgatacaacaattacaatattcgtaaaactacgataatcacttgaaaatattcgtaaaactacgatacaactggaacaatattcgtaaaactacga

SEQ ID NO: 51 - DNA, 114 bp, synthetic construct
  source 1..114: mol_type = other DNA
catgaagttttcagacataaatacaacaattcatgaagttttcagacataaataatcacttgcatgaagttttcagacataaatacaactggacatgaagttttcagacataaa

SEQ ID NO: 52 - DNA, 114 bp, synthetic construct
  source 1..114: mol_type = other DNA
catgaagttttctgacataaatacaacaattcatgaagttttctgacataaataatcacttgcatgaagttttctgacataaatacaactggacatgaagttttctgacataaa

SEQ ID NO: 53 - DNA, 243 bp, synthetic construct
  source 1..243: mol_type = other DNA
catgaagttttcagacataaatacaacaattcatgaagttttcagacataaataatcacttgcatgaagttttcagacataaatacaactggacatgaagttttcagacataaataactagttctttgacatgaagttttcagacataaatacaacaattcatgaagttttcagacataaataatcacttgcatgaagttttcagacataaatacaactggacatgaagttttcagacataaa

SEQ ID NO: 54 - DNA, 114 bp, synthetic construct
  source 1..114: mol_type = other DNA
tctttgtttataatatcttgatacaacaatttctttgtttataatatcttgataatcacttgtctttgtttataatatcttgatacaactggatctttgtttataatatcttga

SEQ ID NO: 55 - DNA, 243 bp, synthetic construct
  source 1..243: mol_type = other DNA
tctttgtttataatatcttgatacaacaatttctttgtttataatatcttgataatcacttgtctttgtttataatatcttgatacaactggatctttgtttataatatcttgataactagttctttgatctttgtttataatatcttgatacaacaatttctttgtttataatatcttgataatcacttgtctttgtttataatatcttgatacaactggatctttgtttataatatcttga

SEQ ID NO: 56 - DNA, 114 bp, synthetic construct
  source 1..114: mol_type = other DNA
gtactctgatcataaattgaatacaacaattgtactctgatcataaattgaataatcacttggtactctgatcataaattgaatacaactggagtactctgatcataaattgaa

SEQ ID NO: 57 - DNA, 243 bp, synthetic construct
  source 1..243: mol_type = other DNA
gtactctgatcataaattgaatacaacaattgtactctgatcataaattgaataatcacttggtactctgatcataaattgaatacaactggagtactctgatcataaattgaataactagttctttgagtactctgatcataaattgaatacaacaattgtactctgatcataaattgaataatcacttggtactctgatcataaattgaatacaactggagtactctgatcataaattgaa

SEQ ID NO: 58 - DNA, 243 bp, synthetic construct
  source 1..243: mol_type = other DNA
gtactctgatcagaaattgaatacaacaattgtactctgatcagaaattgaataatcacttggtactctgatcagaaattgaatacaactggagtactctgatcagaaattgaataactagttctttgagtactctgatcagaaattgaatacaacaattgtactctgatcagaaattgaataatcacttggtactctgatcagaaattgaatacaactggagtactctgatcagaaattgaa

SEQ ID NO: 59 - DNA, 114 bp, synthetic construct
  source 1..114: mol_type = other DNA
gtacaagatcatgaaattgaatacaacaattgtacaagatcatgaaattgaataatcacttggtacaagatcatgaaattgaatacaactggagtacaagatcatgaaattgaa

SEQ ID NO: 60 - DNA, 114 bp, synthetic construct
  source 1..114: mol_type = other DNA
ccatgagtaatctcggtttgatacaacaattccatgagtaatctcggtttgataatcacttgccatgagtaatctcggtttgatacaactggaccatgagtaatctcggtttga

SEQ ID NO: 61 - DNA, 114 bp, synthetic construct
  source 1..114: mol_type = other DNA
ccatgagtaatcacggtttgatacaacaattccatgagtaatcacggtttgataatcacttgccatgagtaatcacggtttgatacaactggaccatgagtaatcacggtttga

SEQ ID NO: 62 - DNA, 114 bp, synthetic construct
  source 1..114: mol_type = other DNA
ccatgagtactaacggtttgatacaacaattccatgagtactaacggtttgataatcacttgccatgagtactaacggtttgatacaactggaccatgagtactaacggtttga

SEQ ID NO: 63 - DNA, 243 bp, synthetic construct
  source 1..243: mol_type = other DNA
ccatgagtaatctcggtttgatacaacaattccatgagtaatctcggtttgataatcacttgccatgagtaatctcggtttgatacaactggaccatgagtaatctcggtttgataactagttctttgaccatgagtaatctcggtttgatacaacaattccatgagtaatctcggtttgataatcacttgccatgagtaatctcggtttgatacaactggaccatgagtaatctcggtttga

SEQ ID NO: 64 - DNA, 114 bp, synthetic construct
  source 1..114: mol_type = other DNA
ccattctatctaacggtttgatacaacaattccattctatctaacggtttgataatcacttgccattctatctaacggtttgatacaactggaccattctatctaacggtttga

SEQ ID NO: 65 - DNA, 114 bp, synthetic construct
  source 1..114: mol_type = other DNA
cttttctaggtaatttagcaatacaacaattcttttctaggtaatttagcaataatcacttgcttttctaggtaatttagcaatacaactggacttttctaggtaatttagcaa

SEQ ID NO: 66 - DNA, 243 bp, synthetic construct
  source 1..243: mol_type = other DNA
cttttctaggtaatttagcaatacaacaattcttttctaggtaatttagcaataatcacttgcttttctaggtaatttagcaatacaactggacttttctaggtaatttagcaataactagttctttgacttttctaggtaatttagcaatacaacaattcttttctaggtaatttagcaataatcacttgcttttctaggtaatttagcaatacaactggacttttctaggtaatttagcaa

SEQ ID NO: 67 - DNA, 114 bp, synthetic construct
  source 1..114: mol_type = other DNA
gtaacgtcaaattagatcgaatacaacaattgtaacgtcaaattagatcgaataatcacttggtaacgtcaaattagatcgaatacaactggagtaacgtcaaattagatcgaa

SEQ ID NO: 68 - DNA, 114 bp, synthetic construct
  source 1..114: mol_type = other DNA
gtaacgtcaaataagatcgaatacaacaattgtaacgtcaaataagatcgaataatcacttggtaacgtcaaataagatcgaatacaactggagtaacgtcaaataagatcgaa

SEQ ID NO: 69 - DNA, 243 bp, synthetic construct
  source 1..243: mol_type = other DNA
gtaacgtcaaattagatcgaatacaacaattgtaacgtcaaattagatcgaataatcacttggtaacgtcaaattagatcgaatacaactggagtaacgtcaaattagatcgaataactagttctttgagtaacgtcaaattagatcgaatacaacaattgtaacgtcaaattagatcgaataatcacttggtaacgtcaaattagatcgaatacaactggagtaacgtcaaattagatcgaa

SEQ ID NO: 70 - DNA, 114 bp, synthetic construct
  source 1..114: mol_type = other DNA
tccagcctttcttaaaacttatacaacaatttccagcctttcttaaaacttataatcacttgtccagcctttcttaaaacttatacaactggatccagcctttcttaaaactta

SEQ ID NO: 71 - DNA, 114 bp, synthetic construct
  source 1..114: mol_type = other DNA
tccaacctttcttaaaacttatacaacaatttccaacctttcttaaaacttataatcacttgtccaacctttcttaaaacttatacaactggatccaacctttcttaaaactta

SEQ ID NO: 72 - DNA, 114 bp, synthetic construct
  source 1..114: mol_type = other DNA
tcccacctttcttaaaacttatacaacaatttcccacctttcttaaaacttataatcacttgtcccacctttcttaaaacttatacaactggatcccacctttcttaaaactta

SEQ ID NO: 73 - DNA, 243 bp, synthetic construct
  source 1..243: mol_type = other DNA
ataccaagactcaatagatgatacaacaattataccaagactcaatagatgataatcacttgataccaagactcaatagatgatacaactggaataccaagactcaatagatgataactagttctttgaataccaagactcaatagatgatacaacaattataccaagactcaatagatgataatcacttgataccaagactcaatagatgatacaactggaataccaagactcaatagatga

SEQ ID NO: 74 - DNA, 243 bp, synthetic construct
  source 1..243: mol_type = other DNA
ataccaagactccatagatgatacaacaattataccaagactccatagatgataatcacttgataccaagactccatagatgatacaactggaataccaagactccatagatgataactagttctttgaataccaagactccatagatgatacaacaattataccaagactccatagatgataatcacttgataccaagactccatagatgatacaactggaataccaagactccatagatga

SEQ ID NO: 75 - RNA, 21 nt, synthetic construct
  source 1..21: mol_type = other RNA
tcgtagtttatgcttatgttg

SEQ ID NO: 76 - RNA, 21 nt, synthetic construct
  source 1..21: mol_type = other RNA
tttatgtcactttgtagatta

SEQ ID NO: 77 - RNA, 21 nt, synthetic construct
  source 1..21: mol_type = other RNA
tttatgtcactttgtagatca

SEQ ID NO: 78 - RNA, 21 nt, synthetic construct
  source 1..21: mol_type = other RNA
tcaagatattataaacttttc

SEQ ID NO: 79 - RNA, 21 nt, synthetic construct
  source 1..21: mol_type = other RNA
tcaagatattataaacttctc

SEQ ID NO: 80 - RNA, 21 nt, synthetic construct
  source 1..21: mol_type = other RNA
ttcaatttcatgatctttacg

SEQ ID NO: 81 - RNA, 21 nt, synthetic construct
  source 1..21: mol_type = other RNA
tcaaaccgttagatagatgct

SEQ ID NO: 82 - RNA, 21 nt, synthetic construct
  source 1..21: mol_type = other RNA
tatgaaactgcttgataagag

SEQ ID NO: 83 - RNA, 21 nt, synthetic construct
  source 1..21: mol_type = other RNA
ttgctaaattacaccatttct

SEQ ID NO: 84 - RNA, 21 nt, synthetic construct
  source 1..21: mol_type = other RNA
ttcgatctttaaatctcaacg

SEQ ID NO: 85 - RNA, 21 nt, synthetic construct
  source 1..21: mol_type = other RNA
taagttttaagaaaggtgttg

SEQ ID NO: 86 - RNA, 21 nt, synthetic construct
  source 1..21: mol_type = other RNA
tcatctatgcttaagattacc

SEQ ID NO: 87 - RNA, 21 nt, synthetic construct
  source 1..21: mol_type = other RNA
tagataagcattataattcct